SEQUENCE LISTING (1 sequence)

SEQ ID NO: 1 - protein, 4 aa, Synthetic construct
  source 1..4: note = amino acid spacer
GGFG

(19) United States
(12) Patent Application Publication
(10) Pub. No.: US 2026/0315612 A1
(43) Pub. Date: Oct. 15, 2026

(54) Topoisomerase Inhibitors, Methods of Making, and Methods of Use Thereof

(71) Applicant: NJ Bio, Inc., Princeton, NJ (US)

(72) Inventors: Nareshkumar Jain, Princeton, NJ (US); Steven Murkli, Jr., Princeton, NJ (US); Nabin Panth, Princeton, NJ (US); Arti Bhujade, Princeton, NJ (US); Richa Vijay Vartak, Princeton, NJ (US); Zachary Sparta, Princeton, NJ (US)

(21) Appl. No.: 19/485,753

(22) PCT Filed: Apr. 8, 2024
(86) PCT No.: PCT/US2024/023548

§ 371 Date: Nov. 19, 2025

Related U.S. Application Data
(60) Provisional application No. 63/469,110, filed on May 26, 2023.
(60) Provisional application No. 63/469,656, filed on May 30, 2023.

(51) Int. Cl. A61K 47/68; A61P 35/00

(52) U.S. Cl. CPC A61K 47/68037; A61K 47/6855; A61P 35/00

(57) ABSTRACT

Disclosed are topoisomerase inhibitor compounds as cytotoxic agents and further as payloads for antibody-drug conjugates. Also disclosed are methods of making and use of the compounds in the treatment of proliferative diseases.

### CROSS-REFERENCE TO RELATED APPLICATIONS

This application is a U.S. National Stage application of PCT/US2024/023548, filed Apr. 8, 2024 which claims the benefit of priority of, U.S. Provisional Patent Application No. 63/469,110, filed May 26, 2023 and U.S. Provisional Patent Application No. 63/469,656, filed May 30, 2023, the contents of which are incorporated herein by reference in their entirety for all purposes.

### SEQUENCE LISTING

The Instant Application contains a Sequence Listing which has been submitted electronically in XML format and is hereby incorporated by reference in its entirety. Said XML copy, created on Nov. 17, 2025, is named “NJB0007US3” and is 2,160 bytes in size. The Sequence Listing does not go beyond the disclosure in the application as filed.

### BACKGROUND

DNA topoisomerases are essential proteins that control DNA topology. Type I topoisomerases catalyze the formation and re-ligation of single-stranded (ss) breaks and Type II catalyze the formation and re-ligation of double-stranded (ds) DNA breaks. Human DNA topoisomerase I remains an important drug target for the treatment of cell proliferation diseases. Camptothecin is a well-known inhibitor of DNA topoisomerase I but suffers from solubility and stability issues. Derivatives and analogs of camptothecin, including irinotecan, topotecan, exatecan and its derivative deruxtecan, are also potent topoisomerase I inhibitors.

Antibody drug conjugates (ADC) include monoclonal antibodies (mAbs) attached to biologically active drugs using chemical linkers with labile bonds. The combination of cytotoxic active agents with the targeting capability of mAbs allows for the specific targeting of cancer cells.

Thus, there remains a need for novel, potent topoisomerase inhibitors and payloads for the development of ADCs for the potential use in the treatment of proliferative diseases, including use in cancer therapy.

### SUMMARY

Disclosed, in various non-limiting embodiments are topoisomerase inhibitors, methods of making, use of the inhibitors as payloads for ADCs, preparation of the ADCs, intermediate compounds, and ADCs formulated into pharmaceutical formulations; methods of use of the topoisomerase inhibitors, intermediate compounds, and ADCs for treatment of proliferative disorders, particularly in cancer therapy.

A compound of Formula (1), or a pharmaceutically acceptable salt thereof,

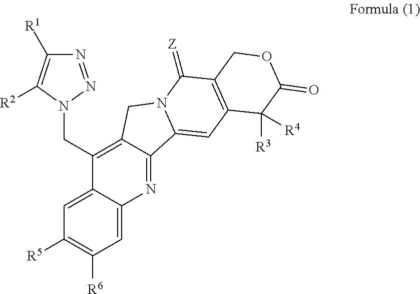

wherein
Z is O or S;
each of R¹ and R² independently is H, optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-;
or
each of R¹ and R² independently is Y⁴—Y³—Y²—Y¹—, wherein
Y¹ is a bond, aryl, heteroaryl, or C₁₋₃ alkyl;
Y² is a bond, O, S, N, or NH, wherein when Y² is N then each of R¹ and R² independently is (Y⁴—Y³)₂—N—Y¹—, or the N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, R², Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, or H₂N—(C₁₋₂ alkyl)-;
each of R³, R⁴, R⁵, and R⁶ independently is H, optionally substituted C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, optionally substituted C₃-C₈ cycloalkyl, optionally substituted C₄-C₈ cycloalkenyl, optionally substituted aryl, optionally substituted heteroaryl, or optionally substituted C₃-C₇ heterocyclic, wherein each optionally substituted group of R³, R⁴, R⁵, or R⁶ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, or H₂N—(C₁₋₂ alkyl)-; and
further wherein any O, S, or N is optionally protected with a suitable protecting group.

In another embodiment, a compound of Formula (A) or Formula (B)

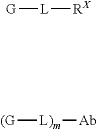

wherein
L is a linking group;
G is a structure of Formula (1) of any one of claims 1-9 or a structure found in Table 01, covalently attached to L through one of R¹, R², R³, R⁴, R⁵, or R⁶, specifically attached through R¹ or R²;
R^{X} is a reactive group suitable for forming a covalent bond to an antibody or antibody fragment;
Ab is an antibody or antibody fragment; and
m is 1, 2, 3, 4, 5, 6, 7, or 8.

In another embodiment, a pharmaceutical composition comprises the compound of Formula (1) or a pharmaceutically acceptable salt thereof, a compound of Formula (A), or a compound of Formula (B), and a pharmaceutically acceptable excipient.

In yet another embodiment, a method of treating a proliferative disease including a cancer, comprises administering to a patient in need thereof a compound of Formula (1) or a pharmaceutically acceptable salt thereof, a compound of Formula (A), a compound of Formula (B), or a pharmaceutical formulation thereof.

These and other features and characteristics are more particularly described below.

### DETAILED DESCRIPTION

Disclosed are topoisomerase inhibitors, derivatives, and antibody drug conjugates of the novel compounds. The compounds, derivatives, and antibody drug conjugates find use as antiproliferative agents specifically as anticancer agents, optionally in the form of cytotoxic payloads for ADCs.

A compound of Formula (1), or a pharmaceutically acceptable salt thereof,

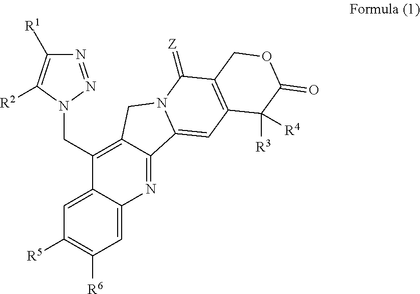

wherein
Z is O or S;
each of R¹ and R² independently is H, optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-;
or
each of R¹ and R² independently is Y⁴—Y³—Y²—Y¹—, wherein
Y¹ is a bond, aryl (e.g. phenyl), heteroaryl (e.g., pyridyl), or C₁₋₃ alkyl;
Y² is a bond, O, S, N, or NH, wherein when Y² is N then each of R¹ and R² independently is (Y⁴—Y³)₂—N—Y¹—, or the N forms an optionally substituted C₅-C₇ heterocyclic with Y⁴ (e.g. piperidinyl, morpholinyl, piperazinyl, pyrrolidinyl);
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, R², Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₂ alkyl)-(C═O)—NH—, or H₂N—(C₁₋₂ alkyl)-(C═O)—NH—;
each of R³, R⁴, R⁵, and R⁶ independently is H, optionally substituted C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, optionally substituted C₃-C₈ cycloalkyl, optionally substituted C₄-C₈ cycloalkenyl, optionally substituted aryl, optionally substituted heteroaryl, or optionally substituted C₃-C₇ heterocyclic, wherein each optionally substituted group of R³, R⁴, R⁵, or R⁶ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—; and further wherein any O, S, or N is optionally protected with a suitable protecting group.

In an embodiment, the compound of Formula (1), or a pharmaceutically acceptable salt thereof, wherein R⁵ is C₁₋₆ alkyl and specifically R⁵ is C₁ alkyl.

In an embodiment, the compound of Formula (1), or a pharmaceutically acceptable salt thereof, wherein R⁶ is halogen and specifically R⁶ is F.

In an embodiment, the compound of Formula (1), or a pharmaceutically acceptable salt thereof, wherein R³ is hydroxyl, C₁₋₃ alkyl, or C₁ haloalkyl, and specifically R³ is hydroxyl.

In an embodiment, the compound of Formula (1), or a pharmaceutically acceptable salt thereof, wherein R⁴ is C₁₋₃ alkyl or C₁₋₃ haloalkyl, and specifically R⁴ is C₂ alkyl.

A compound of Formula (1) includes a compound of Formula (1a), or a pharmaceutically acceptable salt thereof,

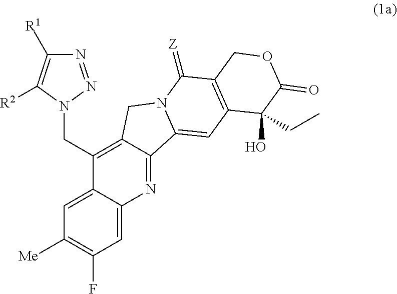

wherein Z, R¹, and R² are as previously defined.

In an embodiment, the compound of Formula (1), Formula (1a), or a pharmaceutically acceptable salt thereof, wherein R² is H and R¹ is optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-; or
R¹ is Y⁴—Y³—Y²—Y¹—, wherein
Y¹ is a bond, aryl, heteroaryl, or C₁₋₃ alkyl;
Y² is a bond, O, S, N, or NH, wherein when Y² is N then R¹ is (Y⁴—Y³)₂—N—Y¹—, or the N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—; and
further wherein any O, S, or N is optionally protected with a suitable protecting group.

In an embodiment, the compound of Formula (1), Formula (1a), or a pharmaceutically acceptable salt thereof, wherein R¹ is H and R² is optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-; or
R² is Y⁴—Y³—Y²—Y¹—, wherein
Y¹ is a bond, aryl, heteroaryl, or C₁₋₃ alkyl;
Y² is a bond, O, S, N, or NH, wherein when Y² is N then R² is (Y⁴—Y³)₂—N—Y¹—, or the N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—; and
further wherein any O, S, or N is optionally protected with a suitable protecting group.

In an embodiment, the compound of Formula (1), Formula (1a), or a pharmaceutically acceptable salt thereof, wherein one of R¹ and R² is H and the other is HO—(C₁₋₂ alkyl)-, H₂N—(C₁₋₂ alkyl)-, optionally substituted phenyl, or Y⁴—Y³—Y²—Y¹—, wherein
Y¹ is a bond, aryl, heteroaryl, or C₁₋₃ alkyl;
Y² is a bond, O, N, or NH, wherein when Y² is N then N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted phenyl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, R², Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—.

In an embodiment, the compound of Formula (1), or a pharmaceutically acceptable salt thereof, as previously described, with the proviso that at least one of R¹ and R² is not H.

Exemplary compounds of Formula (1) and Formula (1a) are provided in Table 01. disclosed herein.

In an embodiment, a compound of Formula (A)

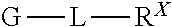

wherein
L is a linking group;
G is a structure of Formula (1) or (1a) covalently attached to L through one of R¹, R², R³, R⁴, R⁵, or R⁶, specifically linked through R¹ or R²; and
R^{X} is a reactive group suitable for forming a covalent bond to an antibody or antibody fragment.

L, the linking group, can be a bond or a group containing 1 to about 250 non-hydrogen atoms including C, N, O, S, halogen, or a combination thereof, further wherein L can optionally include one or more groups including an ether, thioether, amide, carbonyl, ester, carbonate, carbamate, urea, or a combination thereof. In an embodiment, L comprises an ethylene glycol unit, specifically about 2 to about 25 repeating ethylene glycol units, more specifically about 5 to about 10 repeating ethylene glycol units; an amino acid unit, specifically 1 to about 12 amino acid units, more specifically about 2 to about 10 amino acid units, and yet more specifically about 2 to about 3 amino acid units; or a combination thereof.

Within the amino acid groups of L, each amino acid unit can be arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, or citrulline (Cit). The amino acid unit may be enzymatically cleaved by one or more enzymes, including a tumor-associated protease. In an embodiment, the amino acid unit is alanine-valine, valine-alanine, valine-citrulline, or citrulline-valine.

In an embodiment, the L linking group comprises amino acid sequence GGFG (SEQ ID NO:1).

The R^{X} group is a reactive group capable of attaching a compound such as Formula (1) or (1a) to an antibody or antibody fragment. The R^{X} may be an amine, —O—NH₂, maleimide, azide, 2,5-dioxopyrrolidin-1-yl formate,

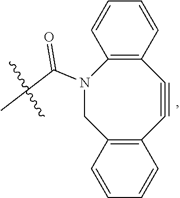 [SMILES: *C(=O)N1Cc2ccccc2C#Cc2ccccc21]

thiol, pentafluorophenyl ester, and the like.

In an embodiment, a compound of Formula (B)

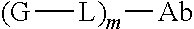

wherein
L is a linking group as previously described;
G is a structure of Formula (1) or (1a) covalently attached to L through one of R¹, R², R³, R⁴, R⁵, or R⁶, specifically linked through R¹ or R²;
Ab is an antibody or antibody fragment; and
m is 1, 2, 3, 4, 5, 6, 7, or 8.

Ab is an antibody or antibody fragment that preferentially binds to a target cell. “Antibody” as herein includes monoclonal antibodies, polyclonal antibodies, dimers, multimers, multispecific antibodies (e.g., bispecific antibodies), and antibody fragments that exhibit the desired biological activity. Antibodies can be murine, human, humanized, chimeric, or derived from other species. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass of immunoglobulin molecule. The immunoglobulins can be derived from any species, including human, murine, or rabbit origin.

In an embodiment, the antibody-drug conjugate of Formula (B) has a drug loading of drug, e.g. compound of Formula (1) or (1a), to antibody/antibody fragment (Ab) of from 1 to about 8, specifically about 2 to about 6, more specifically about 3 to about 4.

Exemplary compounds of Formula (A) are compounds 46 and 47 disclosed herein.

Disclosed herein are pharmaceutical formulations comprising a compound of Formula (1) or (1a), compound of Formula (A), or compound of Formula (B), specifically an antibody-drug conjugate of Formula (B).

The compound of Formula (1), Formula (1a), compound of Formula (A), or Formula (B), are cytotoxic compounds suitable for use to treat proliferative diseases, specifically as anticancer agents.

The compounds are described using standard nomenclature. Unless defined otherwise, all technical and scientific terms used herein have the same meaning as is commonly understood by one of ordinary skill in the art to which this disclosure belongs. Unless clearly contraindicated by the context each compound name includes the free acid or free base form of the compound as well hydrates of the compound and all pharmaceutically acceptable salts of the compound.

The terms “Formula (1),” “Formula (1a),” “Formula (A),” and “Formula (B)”, etc., as used herein, encompass all compounds that satisfy Formula (1), Formula (A), and Formula (B), including any enantiomers, racemates and stereoisomers, as well as all pharmaceutically acceptable salts and radioisotopes of such compounds. The phrases “a compound of Formula (1),” “a compound of Formula (1a),” “a compound of Formula (A),” and “a compound of Formula (B)” include all subgeneric groups of Formula (1), Formula (1a), Formula (A), and Formula (B), and so forth, as well as all forms of such compounds, including salts and hydrates, unless clearly contraindicated by the context in which this phrase is used.

Formula (1) and Formula (1a) include all subformulae thereof. In certain situations, the compounds of Formula (1) and Formula (1a) may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In these situations, single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example, a chiral high performance liquid chromatography (HPLC) column.

Where a compound exists in various tautomeric forms, the compound is not limited to any one of the specific tautomers, but rather includes all tautomeric forms.

All isotopes of atoms occurring in the present compounds are contemplated. Isotopes include those atoms having the same atomic number but different mass numbers. By way of general example, and without limitation, isotopes of hydrogen include tritium and deuterium; isotopes of carbon include ¹¹C, ¹³C, and ¹⁴C; and an isotope of fluorine includes ¹⁸F.

A dash (“-”) that is not between two letters or symbols is used to indicate a point of attachment for a substituent. For example, —(CH₂)C₃-C₈cycloalkyl is attached through carbon of the methylene (CH₂) group.

“Alkanoyl” is an alkyl group as defined herein, covalently bound to the group it substitutes by a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

The term “alkyl”, as used herein, means a branched or straight chain saturated aliphatic hydrocarbon group having the specified number of carbon atoms, generally from 1 to about 12 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₆alkyl, C₁-C₄alkyl, and C₁-C₂alkyl. When C₀-C₁ alkyl is used herein in conjunction with another group, for example, (cycloalkyl)C₀-C₄alkyl, the indicated group, in this case cycloalkyl, is either directly bound by a single covalent bond (C₀), or attached by an alkyl chain having the specified number of carbon atoms, in this case 1, 2, 3, or 4 carbon atoms. Examples of alkyl include, but are not limited to, methyl, ethyl, n-propyl, isopropyl, n-butyl, 3-methylbutyl, t-butyl, n-pentyl, and sec-pentyl.

The term “alkoxy” represents an alkyl group as defined above with the indicated number of carbon atoms attached through an oxygen bridge. Examples of alkoxy include methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

The term “aryl”, as used herein, means aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Bicyclic aryl groups may be further substituted with carbon or non-carbon atoms or groups. Bicyclic aryl groups may contain two fused aromatic rings (naphthyl) or an aromatic ring fused to a 5- to 7-membered non-aromatic cyclic group that optionally contains 1 or 2 heteroatoms independently chosen from N, O, and S, for example, a 3,4-methylenedioxy-phenyl group. Aryl groups include, for example, phenyl, naphthyl, including 1-naphthyl and 2-naphthyl, and bi-phenyl.

The term “cycloalkyl”, as used herein, indicates a saturated hydrocarbon ring group, having only carbon ring atoms and having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane.

The term “cycloalkenyl”, as used herein, means a saturated hydrocarbon ring group, comprising one or more unsaturated carbon-carbon bonds, which may occur in any stable point of the ring, and having the specified number of carbon atoms. Monocyclic cycloalkenyl groups typically have from 3 to about 8 carbon ring atoms or from 3 to 7 (3, 4, 5, 6, or 7) carbon ring atoms. Cycloalkenyl substituents may be pendant from a substituted nitrogen or carbon atom, or a substituted carbon atom that may have two substituents may have a cycloalkenyl group, which is attached as a spiro group. Examples of cycloalkenyl groups include cyclopropenyl, cyclobutenyl, cyclopentenyl, or cyclohexenyl as well as bridged or caged saturated ring groups such as norbornene.

The term “heteroaryl”, as used herein, indicates a stable 5- to 7-membered monocyclic or 7- to 10-membered bicyclic heterocyclic ring which contains at least 1 aromatic ring that contains from 1 to 4, or specifically from 1 to 3, heteroatoms chosen from N, O, and S, with remaining ring atoms being carbon. When the total number of S and O atoms in the heteroaryl group exceeds 1, these heteroatoms are not adjacent to one another. Specifically, the total number of S and O atoms in the heteroaryl group is not more than 2, more specifically the total number of S and O atoms in the heteroaryl group is not more than 1. A nitrogen atom in a heteroaryl group may optionally be quaternized. When indicated, such heteroaryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a [1,3]dioxolo[4,5-c]pyridyl group. In certain embodiments 5- to 6-membered heteroaryl groups are used. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, and 5,6,7,8-tetrahydroisoquinoline.

“Haloalkyl” includes both branched and straight-chain alkyl groups having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined herein attached through an oxygen bridge (oxygen of an alcohol radical).

“Halo” or “halogen” is any of fluoro, chloro, bromo, and iodo.

“Mono- and/or di-alkylamino” is a secondary or tertiary alkyl amino group, wherein the alkyl groups are independently chosen alkyl groups, as defined herein, having the indicated number of carbon atoms. The point of attachment of the alkylamino group is on the nitrogen. Examples of mono- and di-alkylamino groups include ethylamino, dimethylamino, and methyl-propyl-amino.

The term “substituted”, as used herein, means that any one or more hydrogens on the designated atom or group is replaced with a selection from the indicated group, provided that the designated atom's normal valence is not exceeded. When the substituent is oxo (i.e., ═O) then 2 hydrogens on the atom are replaced. When an oxo group substitutes aromatic moieties, the corresponding partially unsaturated ring replaces the aromatic ring. For example, a pyridyl group substituted by oxo is a pyridone. A stable compound or stable structure is meant to imply a compound that is sufficiently robust to survive isolation from a reaction mixture, and subsequent formulation into an effective therapeutic agent.

Unless otherwise specified, e.g. when a dash (“-”) is used, substituents are named into the core structure. For example, it is to be understood that when (cycloalkyl)alkyl is listed as a possible substituent the point of attachment of this substituent to the core structure is in the alkyl portion, or when arylalkyl is listed as a possible substituent the point attachment to the core structure is the alkyl portion.

Suitable groups that may be present on a “substituted” or “optionally substituted” position include, but are not limited to, halogen; cyano; hydroxyl; nitro; azido; alkanoyl (such as a C₂-C₆ alkanoyl group such as acyl or the like); carboxamido; alkyl groups (including cycloalkyl groups) having 1 to about 8 carbon atoms, or 1 to about 6 carbon atoms; alkenyl and alkynyl groups including groups having one or more unsaturated linkages and from 2 to about 8, or 2 to about 6 carbon atoms; alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being an exemplary arylalkyl group; arylalkoxy having 1 to 3 separate or fused rings and from 6 to about 18 ring carbon atoms, with benzyloxy being an exemplary arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothienyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

The term “pharmaceutically acceptable salt”, as used herein, includes derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The compounds of Formula (1), Formula (1a), and their conjugates may be formulated with one or more excipients and prepared into pharmaceutical formulations for any suitable route of administration including oral or parenteral administration. As used herein, parenteral administration includes intravenous, cutaneous, subcutaneous, intramuscular, and the like.

The pharmaceutical composition generally comprises the compound of Formula (1), Formula (1a), or conjugates thereof, and a pharmaceutically acceptable excipient, including carriers, buffers, antioxidants, and the like.

Pharmaceutical formulations for oral administration include tablets, capsules, powders, liquid, semisolids, and the like. Known pharmaceutically acceptable excipients used for oral administration may be used.

For parenteral administration, including intravenous or other injection, the formulation will comprise the compound of Formula (1), Formula (1a), or a conjugate thereof, and a parenterally acceptable aqueous solution. Parenteral solutions are to be pyrogen-free and have suitable pH, isotonicity, and stability. Suitable parenteral vehicles include Sodium Chloride Injection, Ringer's Injection, Lactated Ringer's Injection, and the like. The parenteral formulation may further comprise an antioxidant, a buffer, a preservative, a stabilizer, or a combination thereof.

The compounds and compositions disclosed herein find use for treating cancer, and in particular, in the form of ADCs for cancer therapy.

“Cancer” as used herein can refer to adenocarcinomas, carcinomas, leukemias, lymphomas, sarcomas, solid and lymphoid cancers, and the like. Examples of different types of cancer include, acute lymphocytic leukemia (acute lymphoblastic leukemia), acute myeloid leukemia (acute myelogenous leukemia, acute myeloblastic leukemia, acute myelocytic leukemia, acute granulocytic leukemia, and acute nonlymphocytic leukemia), anal cancer, B-cell lymphoma, bile duct cancer, bladder cancer, blood cancer, bone cancer, breast cancer, Burkitt's lymphoma, central nervous system cancer, cervical cancer, choriocarcinoma, chronic lymphocytic leukemia, chronic myeloid leukemia (chronic myelogenous leukemia), colon cancer, colorectal cancer, endometrial cancer, epithelial cancer, esophageal cancer, fibrosarcoma, gall bladder cancer, gastrointestinal carcinoid tumors, glioma, head and neck cancer, Large Cell lymphoma, liposarcoma, liver cancer (i.e., hepatocarcinoma), lung cancer (e.g., non-small cell lung cancer or NSCLC), melanoma, monocytic leukemia, multiple myeloma, myelodysplastic syndromes (MDS), myelogenous leukemia, neuroblastoma, non-Hodgkin's lymphoma, ovarian cancer, osteogenic sarcoma, pancreatic cancer, pleural cancer, prostate cancer, rectal cancer, renal cancer (i.e., renal cell carcinoma), skin cancer, Small Cell lymphoma, small intestine cancer, stomach (gastric) cancer, testicular cancer, thyroid cancer, uterine cancer, and the like.

The patient may be a mammalian patient, specifically a human.

The dosage amount of the compound of Formula (1) or Formula (1a) for the treatment of a disease, such as a proliferative disease, will depend on the disease to be treated, the severity of the disease, previous therapy and concurrent therapy, the patient's clinical history, and other factors. The Formula (1), Formula (1a), or a conjugate thereof, can be administered to the patient at one time or over a series of treatments over days, weeks, or months. In an exemplary amount, about 1 g/kg to about 15 mg/kg of the compound of Formula (1) or Formula (1a) may be a starting dosage for administration to the patient. The dosage may be by one or more separate administrations, or by continuous infusion. An exemplary daily dosage might range from about 1 g/kg to about 100 mg/kg or more, depending on the factors previously discussed. An exemplary dosage of the compound of Formula (1) or Formula (1a) to be administered to a patient can be in the range of about 0.1 to about 10 mg/kg of patient weight. In an embodiment, an exemplary dosing regimen comprises a course of administering an initial loading dose of about 4 mg/kg, followed by additional doses every week, two weeks, or three weeks of the compound of Formula (1) or Formula (1a).

In an embodiment, a pharmaceutical composition comprises the compound of Formula (1), Formula (1a), or a pharmaceutically acceptable salt thereof, and a pharmaceutically acceptable excipient.

In yet another embodiment, a method of treating a proliferative disease including a cancer, comprises administering to a patient in need thereof a compound of Formula (1), Formula (1a), or a pharmaceutically acceptable salt thereof, or a pharmaceutical formulation thereof.

The following examples are merely illustrative of the topoisomerase inhibitor compounds disclosed herein and are not intended to limit the scope hereof.

### EXAMPLES

General Solvents and reagents were purchased from Sigma-Aldrich, VWR, Ambeed, Combi-Blocks, Inc., or Fisher Scientific, and used without further purification. Reactions were monitored either by thin-layer chromatography (TLC) or by analytical liquid chromatography-mass spectrometry (LC-MS) employing a Waters Acquity Ultra Performance LC system and a Synapt high-definition mass spectrometer. Unless otherwise indicated, compounds were purified by flash column chromatography on a Teledyne ISCO Combi-Flash system using normal phase silica gel (SiliCycle Inc.) or reverse phase (Teledyne Gold-C18, GoldC18Aq, or Gemini (5 μm C18 110 Å150×30 mm) with (5% ACN+0.05% FA)/(H₂O+0.05% FA) to 95% (ACN+0.05% FA)/(H₂O+0.05% FA) gradient).

The purity of compounds was determined by analytical HPLC (Waters Acquity Ultra Performance) using an Acquity UPLC CSH C18 1.7 μm (50 mm×2.1 mm) column and flow rate of 0.3 mL/min. Gradient conditions: solvent A (0.1% formic acid in water) and solvent B (0.1% formic acid in acetonitrile): 0-0.1 min 95% A, 0.1-4.0 min 5-95% B (linear gradient), 4.0-5.0 min 95% B, UV detection at 254 nm and 220 nm.

DMTMM=4-(4,6-dimethoxy-1,3,5-triazin-2-yl)-4-methylmorpholinium chloride, NMM=4-methylmorpholine, DMF=dimethylformamide, TEA=triethylamine, ACN=acetonitrile, FA=formic acid, DIPEA=diisopropylethylamine, HATU=Hexafluorophosphate Azabenzotriazole Tetramethyl Uronium, HOBt=Hydroxybenzotriazole, EEDQ=N-ethoxycarbonyl-2-ethoxy-1,2-dihydroquinoline, PyBOP=Benzotriazol-1-yloxy)tripyrrolidinophosphonium hexafluorophosphate.

### Example 01: Representative Copper (Cu(I))-catalyzed Azide-Alkyne Cycloaddition (CuAAC) Pilot Scale Coupling Reaction to Prepare Compounds 1, 2, 4-6, 11, 13, 16, 19, 22, 23, 33, 36, 37, 39, 46-49, and 50-59

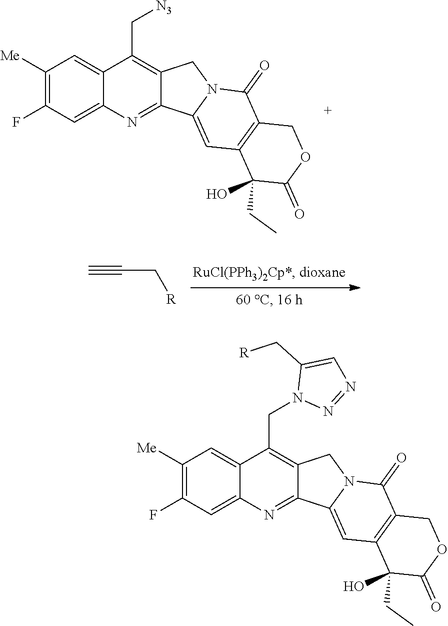

Camptothecin azide (ACS Med. Chem. Lett. 2019, 10, 1386-1392) (10 mg, 0.023 mmol) and alkyne (0.0919 mmol, 4 eq) were added to vial under argon atmosphere. (PPh₃)₃CuBr (4.3 mg, 20 mol %) was added to the vial and dissolved in anhydrous dichloromethane (1.435 mL). Diisopropylethylamine (80 μL, 0.4593 mmol, 20 eq) was added to the vial and the reaction was stirred at 35° C. for 16 hours sealed under argon atmosphere. The crude reaction was concentrated en vacuo and dissolved in (3 mL) (2:1) (ACN:H₂O)+0.05% formic acid (FA). The crude material was purified via reverse phase chromatography (15.5 g C18) (5% ACN+0.05% FA to 95% ACN+0.05% FA/H₂O+0.05% FA gradient). The product was recovered by lyophilization to obtain payloads 1, 2, 4-6, 11, 13, 16, 19, 22, 23, 33, 36, 37, 39, 46-49, and 50-59. Yields and mass spectrometry results are provided in Table 01.

### Example 02: Ruthenium-Catalyzed Azide-Alkyne Cycloaddition (RuAAC) Pilot Scale Coupling Reaction to Prepare Payloads 3, 8, 9, 12, and 65

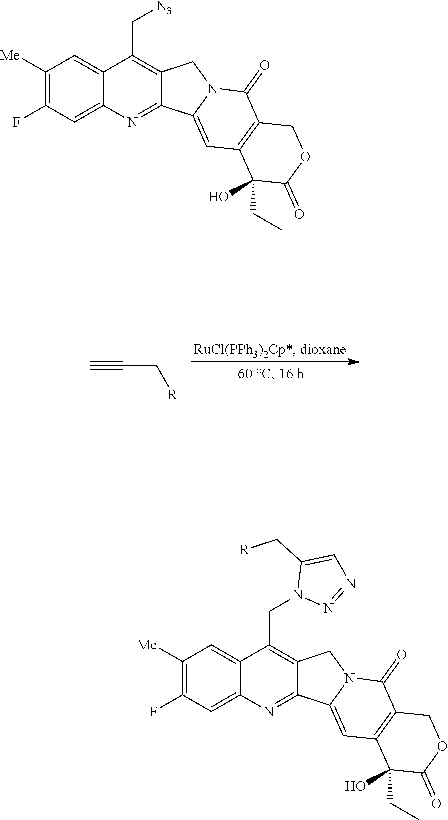

Camptothecin-N₃ (10 mg, 0.023 mmol) and alkyne (0.0919 mmol, 4 eq) were added to vial under argon atmosphere. Cp*RuCl(PPh₃)₂ (3.66 mg, 20 mol %) was added to the vial and dissolved in anhydrous 1,4-dioxane (1.464 mL). The reaction was stirred at 60° C. for 16 hours sealed under argon atmosphere. The crude reaction was concentrated en vacuo and dissolved in (3 mL) (2:1) (ACN:H₂O)+0.05% FA. The crude material was purified via reverse phase chromatography (15.5 g C18) (5% (ACN+0.05% FA) to 95% (ACN+0.05%) FA/(H₂O+0.05% FA) gradient). The product was recovered by lyophilization to obtain compounds 3, 8, 9, 12, and 65. Yields and mass spectrometry results are provided in Table 01.

### Example 03: Representative Glycolic Acid Amide Coupling to Prepare Payloads 7, 10, 15, 21, 38, 60-64, 68, and 69

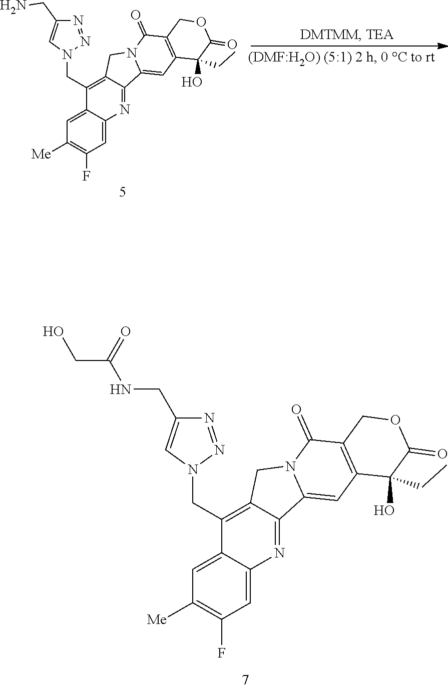 [SMILES: CC[C@@]1(O)C(=O)OCc2c1cc1n(c2=O)Cc2c-1nc1cc(F)c(C)cc1c2Cn1cc(CN)nn1 ; CC[C@@]1(O)C(=O)OCc2c1cc1n(c2=O)Cc2c-1nc1cc(F)c(C)cc1c2Cn1cc(CNC(=O)CO)nn1]

Reference: ACS Med. Chem. Lett. 2019, 10, 1386-1392 To an oven dried vial (7.5 mg, 0.0153 mmol) 5 was added with (1.3 mg, 0.0170 mmol) glycolic acid under argon atmosphere. DMF (0.425 mL) was added to the vial followed by (2 μL, 0.0153 mmol) triethylamine. The vial was cooled to 0° C. In a separate vial (8.67 mg, 0.0313 mmol) DMTMM was dissolved in (0.085 mL) H₂O. The DMTMM solution was added to the reaction and then allowed to warm to room temperature. The vial was stirred at room temperature for 2 h. After completion of the reaction, the vial was diluted with (2 mL) H₂O and (1 mL) of acetonitrile and loaded on a 15.5 g C18Aq column. (0% ACN+0.05% FA) to 50% (ACN+0.05% FA)/(H₂O+0.05% FA) gradient). The product was recovered by lyophilization to obtain 7 (3.6 mg, 43% yield) as a white solid. (ESI) m/z: [M+H]+=549.29. This reaction was used to obtain payloads 7, 10, 15, 21, 38, 60-64, 68, and 69. Yields and mass spectrometry results are provided in Table 01.

### Example 04: Representative Fmoc-Glycine Coupling and Deprotection to Prepare Payloads 32, 66, and 67

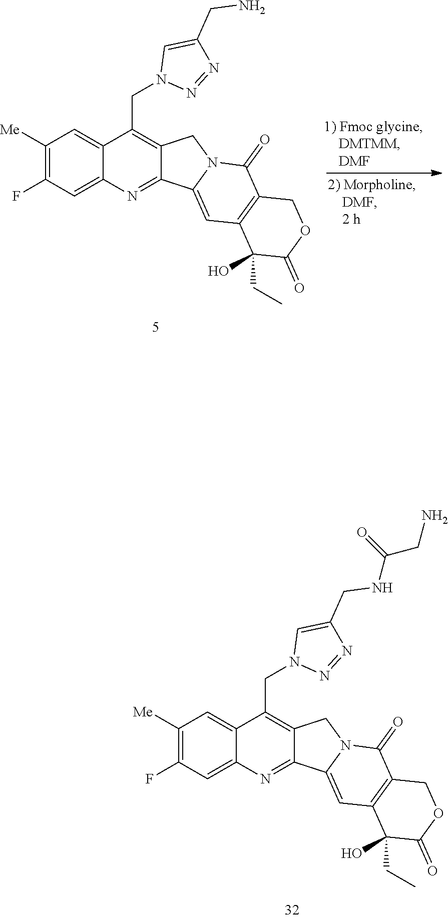 [SMILES: CC[C@@]1(O)C(=O)OCc2c1cc1n(c2=O)Cc2c-1nc1cc(F)c(C)cc1c2Cn1cc(CN)nn1 ; CC[C@@]1(O)C(=O)OCc2c1cc1n(c2=O)Cc2c-1nc1cc(F)c(C)cc1c2Cn1cc(CNC(=O)CN)nn1]

To an oven dried vial was added fmoc-glycine (7.6 mg, 0.025 mmol, 1.25 eq), 5 (10 mg, 0.02 mmol, 1 eq), and DMF (0.36 mL, 0.07 M) at room temperature. DMTMM (11.3 mg, 0.041 mmol, 2 eq) was added and stirred at room temperature until the reaction was completed. The crude reaction was loaded neat on a 15.5 g C18Aq column. (0% ACN+0.05% FA) to 50% (ACN+0.05% FA)/(H₂O+0.05% FA) gradient). The fmoc-protected glycine product was obtained by lyophilization as a white solid. The fmoc-protected glycine product was then dissolved in (1:4) (Morpholine:DMF) (0.056 mL) at room temperature and stirred for 1 h, whereupon the product was obtained by precipitation by addition of Et₂O (0.5 mL). The solid was isolated by centrifugation and washed with Et₂O further (1 mL) by vortex, centrifugation, and decanting to obtain 32 (0.8 mg, 38% yield) as a white solid. (ESI) m/z: [M+H]=548.19. This reaction was used to obtain payloads 32, 66, and 67. Yields and mass spectrometry results are provided in Table 01.

TABLE 01
Payloads

| | | (ESI) m/z: | |
|---|---|---|---|
| Cmpd | Yield | [M + H]⁺ | Structure |
| 1 | 61% | 576.50 | 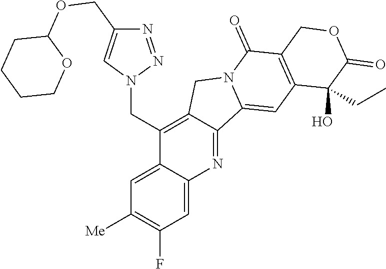 [SMILES: CC[C@@]1(O)C(=O)OCc2c1cc1n(c2=O)Cc2c-1nc1cc(F)c(C)cc1c2Cn1cc(COC2CCCCO2)nn1] |
| 2 | 54% | 491.06 | 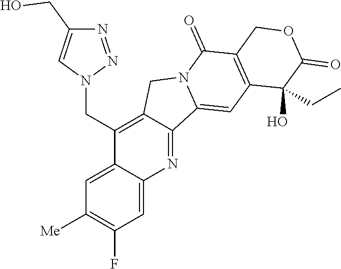 [SMILES: CC[C@@]1(O)C(=O)OCc2c1cc1n(c2=O)Cc2c-1nc1cc(F)c(C)cc1c2Cn1cc(CO)nn1] |
| 3 | 17% | 492.34 | 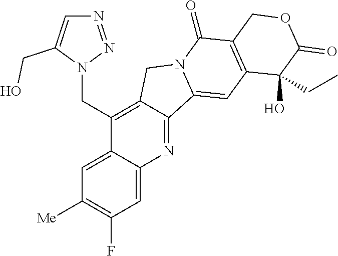 [SMILES: CC[C@@]1(O)C(=O)OCc2c1cc1n(c2=O)Cc2c-1nc1cc(F)c(C)cc1c2Cn1nncc1CO] |
| 4 | 62% | 591.58 | 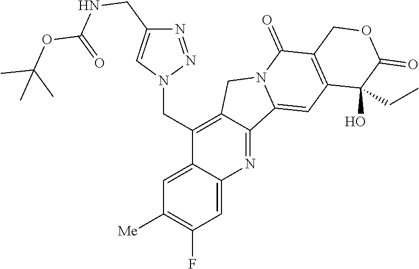 [SMILES: CC[C@@]1(O)C(=O)OCc2c1cc1n(c2=O)Cc2c-1nc1cc(F)c(C)cc1c2Cn1cc(CNC(=O)OC(C)(C)C)nn1] |
| 5 | 44% | 491.17 | 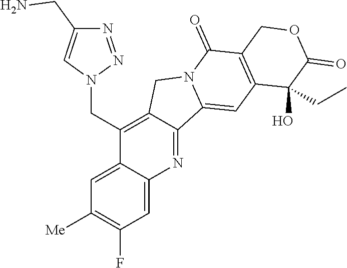 [SMILES: CC[C@@]1(O)C(=O)OCc2c1cc1n(c2=O)Cc2c-1nc1cc(F)c(C)cc1c2Cn1cc(CN)nn1] |
| 6 | 35% | 505.34 | 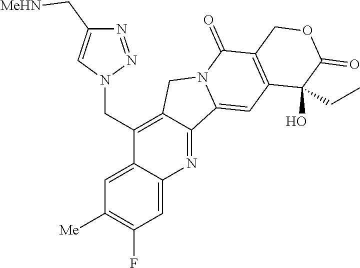 [SMILES: CC[C@@]1(O)C(=O)OCc2c1cc1n(c2=O)Cc2c-1nc1cc(F)c(C)cc1c2Cn1cc(CNC)nn1] |
| 7 | 43% | 549.29 | 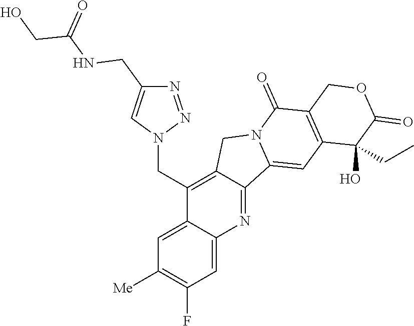 [SMILES: CC[C@@]1(O)C(=O)OCc2c1cc1n(c2=O)Cc2c-1nc1cc(F)c(C)cc1c2Cn1cc(CNC(=O)CO)nn1] |
| 8 | 58% | 491.06 | 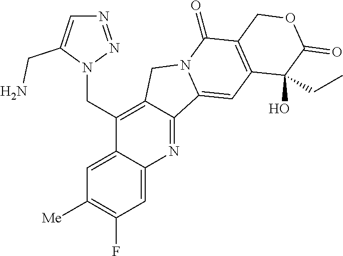 [SMILES: CC[C@@]1(O)C(=O)OCc2c1cc1n(c2=O)Cc2c-1nc1cc(F)c(C)cc1c2Cn1nncc1CN] |
| 9 | 16% | 505.26 | 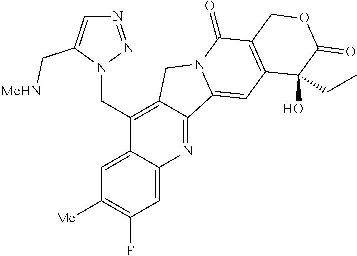 [SMILES: CC[C@@]1(O)C(=O)OCc2c1cc1n(c2=O)Cc2c-1nc1cc(F)c(C)cc1c2Cn1nncc1CNC] |
| 10 | Proposed | Proposed | 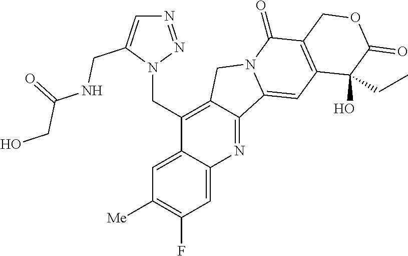 [SMILES: CC[C@@]1(O)C(=O)OCc2c1cc1n(c2=O)Cc2c-1nc1cc(F)c(C)cc1c2Cn1nncc1CNC(=O)CO] |
| 11 | 19% | 506.20 | 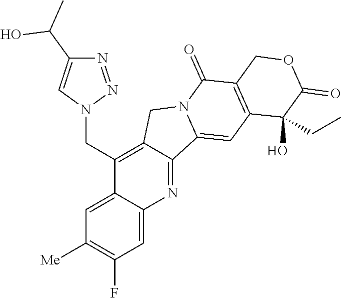 [SMILES: CC[C@@]1(O)C(=O)OCc2c1cc1n(c2=O)Cc2c-1nc1cc(F)c(C)cc1c2Cn1cc(C(C)O)nn1] |
| 12 | 60% | 522.21 | 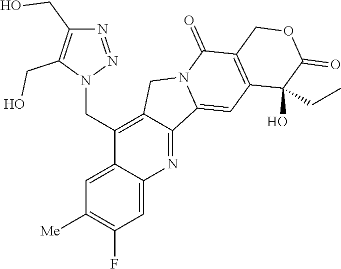 [SMILES: CC[C@@]1(O)C(=O)OCc2c1cc1n(c2=O)Cc2c-1nc1cc(F)c(C)cc1c2Cn1nnc(CO)c1CO] |
| 13 | 64% | 553.24 | 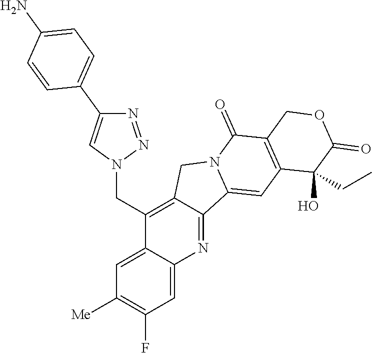 [SMILES: CC[C@@]1(O)C(=O)OCc2c1cc1n(c2=O)Cc2c-1nc1cc(F)c(C)cc1c2Cn1cc(-c2ccc(N)cc2)nn1] |
| 14 | Proposed | Proposed | 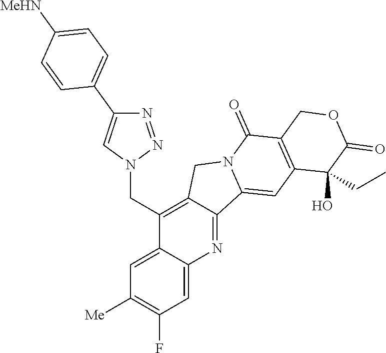 [SMILES: CC[C@@]1(O)C(=O)OCc2c1cc1n(c2=O)Cc2c-1nc1cc(F)c(C)cc1c2Cn1cc(-c2ccc(NC)cc2)nn1] |
| 15 | 46% | 611.28 | 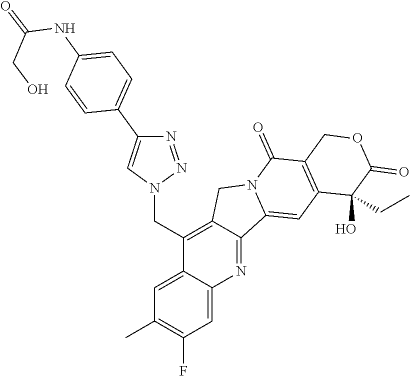 [SMILES: CC[C@@]1(O)C(=O)OCc2c1cc1n(c2=O)Cc2c-1nc1cc(F)c(C)cc1c2Cn1cc(-c2ccc(NC(=O)CO)cc2)nn1] |
| 16 | 83% | 553.24 | 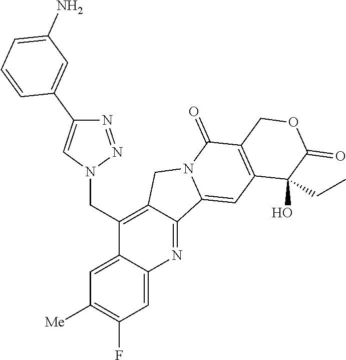 [SMILES: CC[C@@]1(O)C(=O)OCc2c1cc1n(c2=O)Cc2c-1nc1cc(F)c(C)cc1c2Cn1cc(-c2cccc(N)c2)nn1] |
| 17 | Proposed | Proposed | 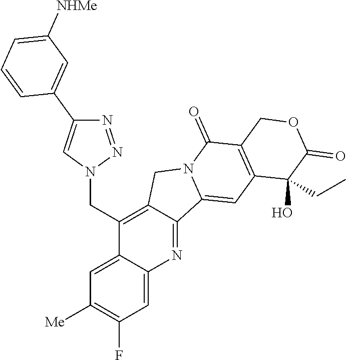 [SMILES: CC[C@@]1(O)C(=O)OCc2c1cc1n(c2=O)Cc2c-1nc1cc(F)c(C)cc1c2Cn1cc(-c2cccc(NC)c2)nn1] |
| 18 | Proposed | Proposed | 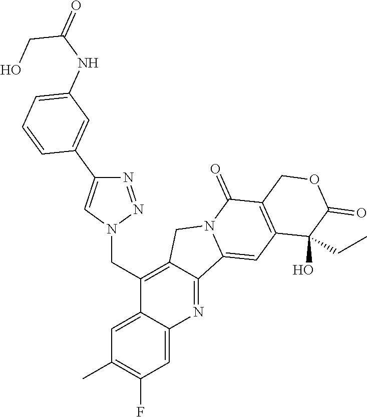 [SMILES: CC[C@@]1(O)C(=O)OCc2c1cc1n(c2=O)Cc2c-1nc1cc(F)c(C)cc1c2Cn1cc(-c2cccc(NC(=O)CO)c2)nn1] |
| 19 | 45% | 553.26 | 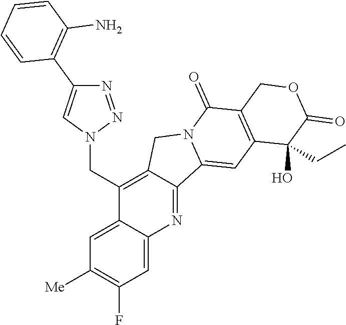 [SMILES: CC[C@@]1(O)C(=O)OCc2c1cc1n(c2=O)Cc2c-1nc1cc(F)c(C)cc1c2Cn1cc(-c2ccccc2N)nn1] |
| 20 | Proposed | Proposed | 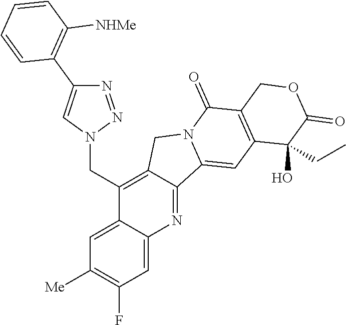 [SMILES: CC[C@@]1(O)C(=O)OCc2c1cc1n(c2=O)Cc2c-1nc1cc(F)c(C)cc1c2Cn1cc(-c2ccccc2NC)nn1] |
| 21 | Proposed | Proposed | 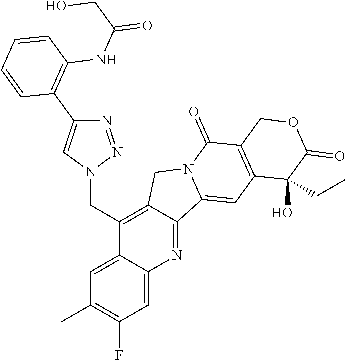 [SMILES: CC[C@@]1(O)C(=O)OCc2c1cc1n(c2=O)Cc2c-1nc1cc(F)c(C)cc1c2Cn1cc(-c2ccccc2NC(=O)CO)nn1] |
| 22 | Quantitative | 560.31 | 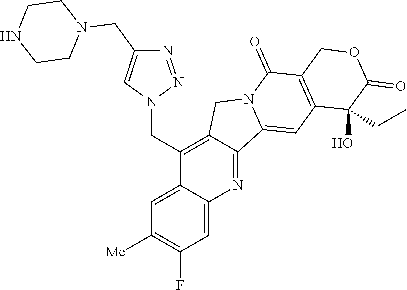 [SMILES: CC[C@@]1(O)C(=O)OCc2c1cc1n(c2=O)Cc2c-1nc1cc(F)c(C)cc1c2Cn1cc(CN2CCNCC2)nn1] |
| 23 | 38% | 589.26 | 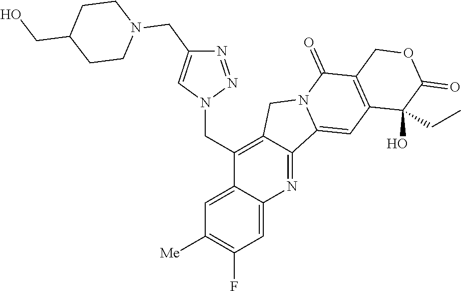 [SMILES: CC[C@@]1(O)C(=O)OCc2c1cc1n(c2=O)Cc2c-1nc1cc(F)c(C)cc1c2Cn1cc(CN2CCC(CO)CC2)nn1] |
| 24 | Proposed | Proposed | |
| 25 | Proposed | Proposed | |
| 26 | Proposed | Proposed | 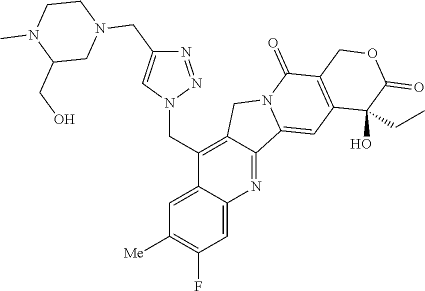 [SMILES: CC[C@@]1(O)C(=O)OCc2c1cc1n(c2=O)Cc2c-1nc1cc(F)c(C)cc1c2Cn1cc(CN2CCN(C)C(CO)C2)nn1] |
| 27 | Proposed | Proposed | 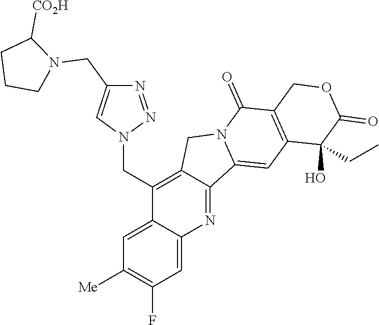 [SMILES: CC[C@@]1(O)C(=O)OCc2c1cc1n(c2=O)Cc2c-1nc1cc(F)c(C)cc1c2Cn1cc(CN2CCCC2C(=O)O)nn1] |
| 28 | Proposed | Proposed | 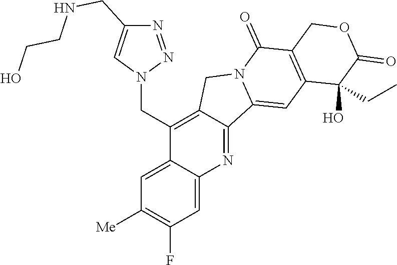 [SMILES: CC[C@@]1(O)C(=O)OCc2c1cc1n(c2=O)Cc2c-1nc1cc(F)c(C)cc1c2Cn1cc(CNCCO)nn1] |
| 29 | Proposed | Proposed | 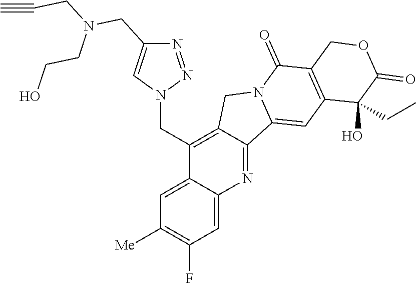 [SMILES: C#CCN(CCO)Cc1cn(Cc2c3c(nc4cc(F)c(C)cc24)-c2cc4c(c(=O)n2C3)COC(=O)[C@]4(O)CC)nn1] |
| 30 | Proposed | Proposed | 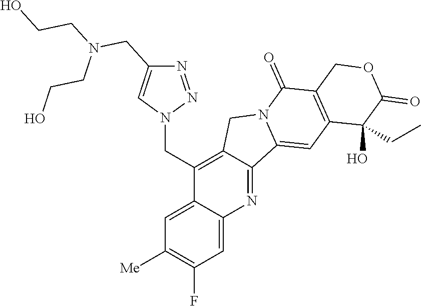 [SMILES: CC[C@@]1(O)C(=O)OCc2c1cc1n(c2=O)Cc2c-1nc1cc(F)c(C)cc1c2Cn1cc(CN(CCO)CCO)nn1] |
| 31 | Proposed | Proposed | 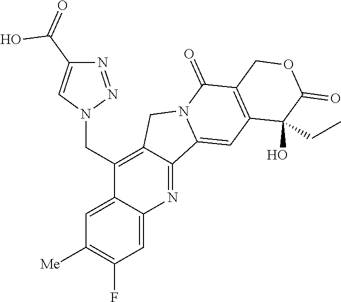 [SMILES: CC[C@@]1(O)C(=O)OCc2c1cc1n(c2=O)Cc2c-1nc1cc(F)c(C)cc1c2Cn1cc(C(=O)O)nn1] |
| 32 | 23% | 548.19 | 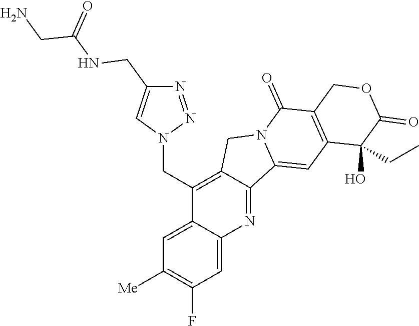 [SMILES: CC[C@@]1(O)C(=O)OCc2c1cc1n(c2=O)Cc2c-1nc1cc(F)c(C)cc1c2Cn1cc(CNC(=O)CN)nn1] |
| 33 | 16% | 574.26 | 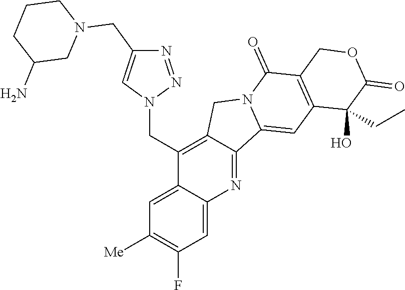 [SMILES: CC[C@@]1(O)C(=O)OCc2c1cc1n(c2=O)Cc2c-1nc1cc(F)c(C)cc1c2Cn1cc(CN2CCCC(N)C2)nn1] |
| 34 | Proposed | Proposed | 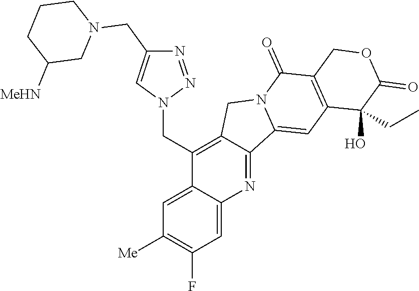 [SMILES: CC[C@@]1(O)C(=O)OCc2c1cc1n(c2=O)Cc2c-1nc1cc(F)c(C)cc1c2Cn1cc(CN2CCCC(NC)C2)nn1] |
| 35 | Proposed | Proposed | 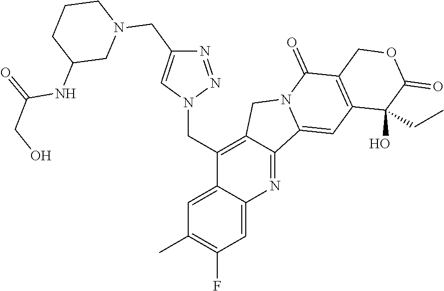 [SMILES: CC[C@@]1(O)C(=O)OCc2c1cc1n(c2=O)Cc2c-1nc1cc(F)c(C)cc1c2Cn1cc(CN2CCCC(NC(=O)CO)C2)nn1] |
| 36 | 21% | 505.28 | 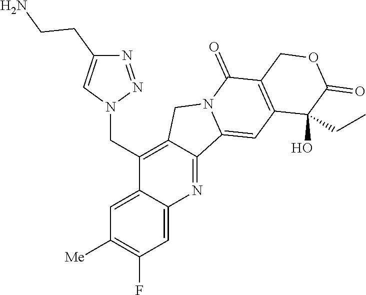 [SMILES: CC[C@@]1(O)C(=O)OCc2c1cc1n(c2=O)Cc2c-1nc1cc(F)c(C)cc1c2Cn1cc(CCN)nn1] |
| 37 | 40% | 519.18 | 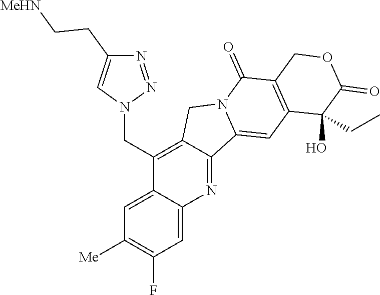 [SMILES: CC[C@@]1(O)C(=O)OCc2c1cc1n(c2=O)Cc2c-1nc1cc(F)c(C)cc1c2Cn1cc(CCNC)nn1] |
| 38 | 47% | 563.35 | 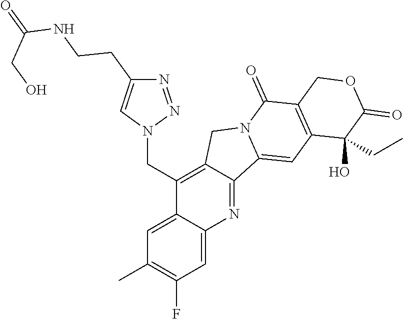 [SMILES: CC[C@@]1(O)C(=O)OCc2c1cc1n(c2=O)Cc2c-1nc1cc(F)c(C)cc1c2Cn1cc(CCNC(=O)CO)nn1] |
| 39 | 39% | 506.15 | 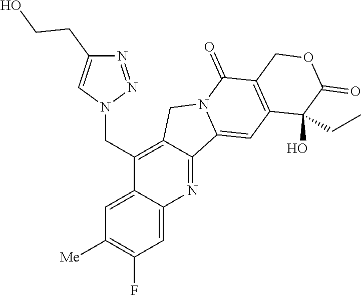 [SMILES: CC[C@@]1(O)C(=O)OCc2c1cc1n(c2=O)Cc2c-1nc1cc(F)c(C)cc1c2Cn1cc(CCO)nn1] |
| 40 | Proposed | Proposed | 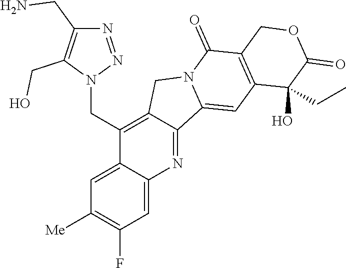 [SMILES: CC[C@@]1(O)C(=O)OCc2c1cc1n(c2=O)Cc2c-1nc1cc(F)c(C)cc1c2Cn1nnc(CN)c1CO] |
| 41 | Proposed | Proposed | 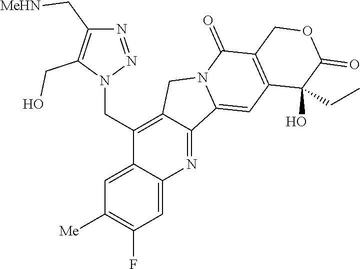 [SMILES: CC[C@@]1(O)C(=O)OCc2c1cc1n(c2=O)Cc2c-1nc1cc(F)c(C)cc1c2Cn1nnc(CNC)c1CO] |
| 42 | Proposed | Proposed | 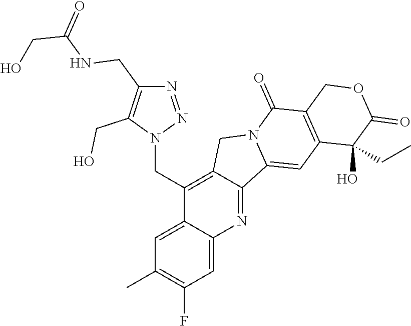 [SMILES: CC[C@@]1(O)C(=O)OCc2c1cc1n(c2=O)Cc2c-1nc1cc(F)c(C)cc1c2Cn1nnc(CNC(=O)CO)c1CO] |
| 43 | Proposed | Proposed | 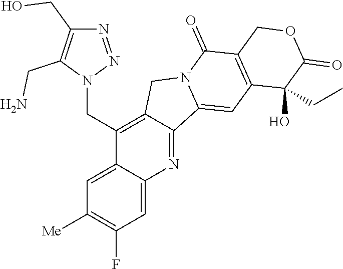 [SMILES: CC[C@@]1(O)C(=O)OCc2c1cc1n(c2=O)Cc2c-1nc1cc(F)c(C)cc1c2Cn1nnc(CO)c1CN] |
| 44 | Proposed | Proposed | 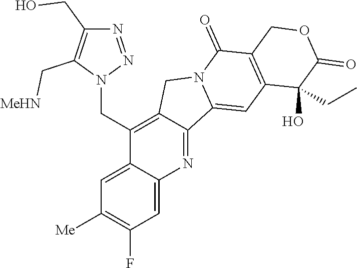 [SMILES: CC[C@@]1(O)C(=O)OCc2c1cc1n(c2=O)Cc2c-1nc1cc(F)c(C)cc1c2Cn1nnc(CO)c1CNC] |
| 45 | Proposed | Proposed | 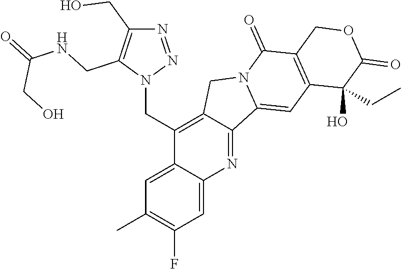 [SMILES: CC[C@@]1(O)C(=O)OCc2c1cc1n(c2=O)Cc2c-1nc1cc(F)c(C)cc1c2Cn1nnc(CO)c1CNC(=O)CO] |
| 46 | 34% | 572.20 | 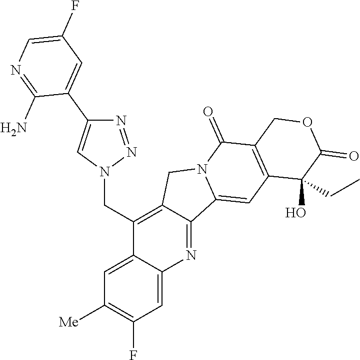 [SMILES: CC[C@@]1(O)C(=O)OCc2c1cc1n(c2=O)Cc2c-1nc1cc(F)c(C)cc1c2Cn1cc(-c2cc(F)cnc2N)nn1] |
| 47 | 38% | 517.37 | 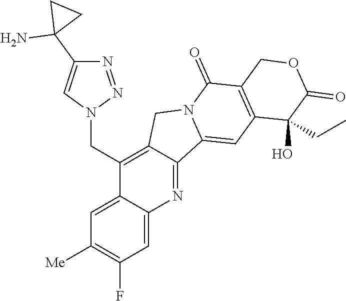 [SMILES: CC[C@@]1(O)C(=O)OCc2c1cc1n(c2=O)Cc2c-1nc1cc(F)c(C)cc1c2Cn1cc(C2(N)CC2)nn1] |
| 48 | 26% | 554.27 | 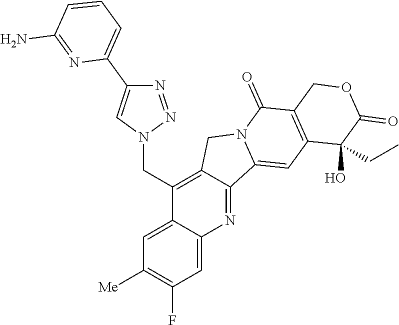 [SMILES: CC[C@@]1(O)C(=O)OCc2c1cc1n(c2=O)Cc2c-1nc1cc(F)c(C)cc1c2Cn1cc(-c2cccc(N)n2)nn1] |
| 49 | 29% | 554.26 | 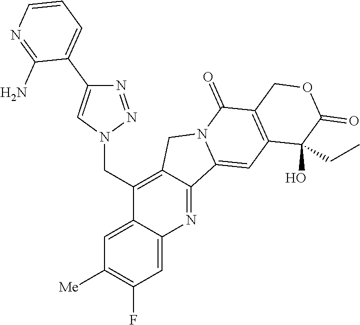 [SMILES: CC[C@@]1(O)C(=O)OCc2c1cc1n(c2=O)Cc2c-1nc1cc(F)c(C)cc1c2Cn1cc(-c2cccnc2N)nn1] |
| 50 | 27% | 505.21 | 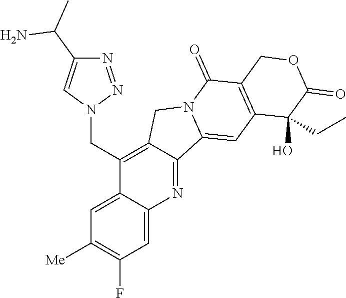 [SMILES: CC[C@@]1(O)C(=O)OCc2c1cc1n(c2=O)Cc2c-1nc1cc(F)c(C)cc1c2Cn1cc(C(C)N)nn1] |
| 51 | 40% | 519.14 | 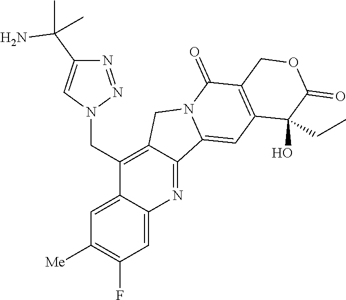 [SMILES: CC[C@@]1(O)C(=O)OCc2c1cc1n(c2=O)Cc2c-1nc1cc(F)c(C)cc1c2Cn1cc(C(C)(C)N)nn1] |
| 52 | 32% | 554.26 | 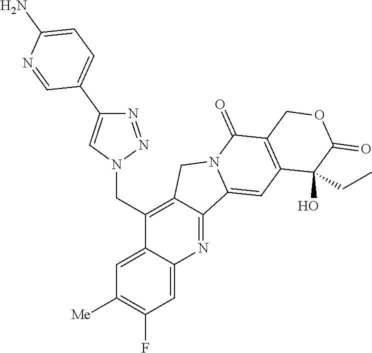 [SMILES: CC[C@@]1(O)C(=O)OCc2c1cc1n(c2=O)Cc2c-1nc1cc(F)c(C)cc1c2Cn1cc(-c2ccc(N)nc2)nn1] |
| 53 | 12% | 519.12 | 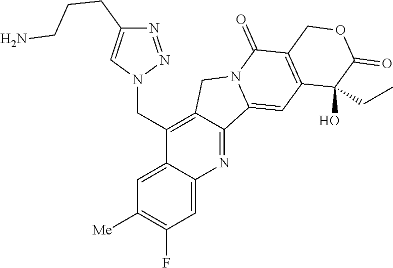 [SMILES: CC[C@@]1(O)C(=O)OCc2c1cc1n(c2=O)Cc2c-1nc1cc(F)c(C)cc1c2Cn1cc(CCCN)nn1] |
| 54 | 21% | 589.27 | 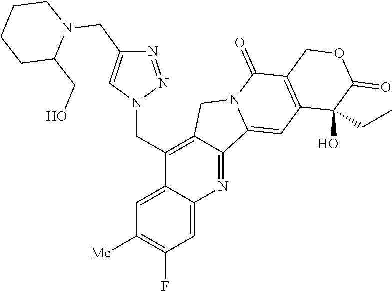 [SMILES: CC[C@@]1(O)C(=O)OCc2c1cc1n(c2=O)Cc2c-1nc1cc(F)c(C)cc1c2Cn1cc(CN2CCCCC2CO)nn1] |
| 55 | 24% | 535.21 | 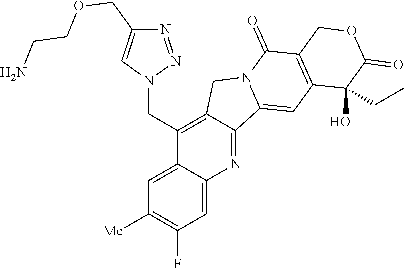 [SMILES: CC[C@@]1(O)C(=O)OCc2c1cc1n(c2=O)Cc2c-1nc1cc(F)c(C)cc1c2Cn1cc(COCCN)nn1] |
| 56 | 32% | 589.27 | 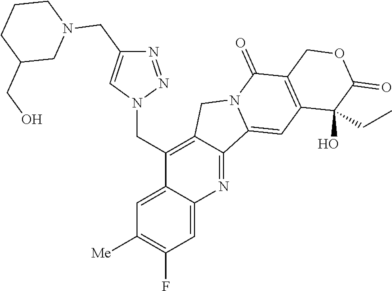 [SMILES: CC[C@@]1(O)C(=O)OCc2c1cc1n(c2=O)Cc2c-1nc1cc(F)c(C)cc1c2Cn1cc(CN2CCCC(CO)C2)nn1] |
| 57 | 26% | 531.25 | 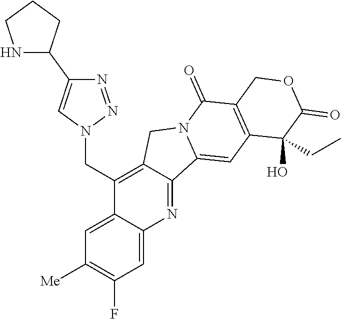 [SMILES: CC[C@@]1(O)C(=O)OCc2c1cc1n(c2=O)Cc2c-1nc1cc(F)c(C)cc1c2Cn1cc(C2CCCN2)nn1] |
| 58 | 25% | 590.20 | 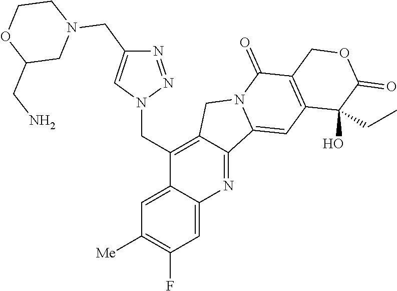 [SMILES: CC[C@@]1(O)C(=O)OCc2c1cc1n(c2=O)Cc2c-1nc1cc(F)c(C)cc1c2Cn1cc(CN2CCOC(CN)C2)nn1] |
| 59 | 40% | 520.08 | 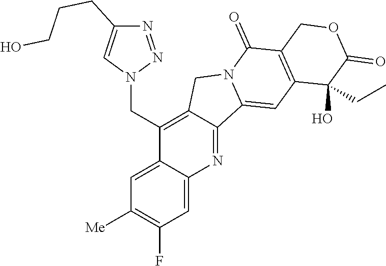 [SMILES: CC[C@@]1(O)C(=O)OCc2c1cc1n(c2=O)Cc2c-1nc1cc(F)c(C)cc1c2Cn1cc(CCCO)nn1] |
| 60 | 75% | 618.14 | 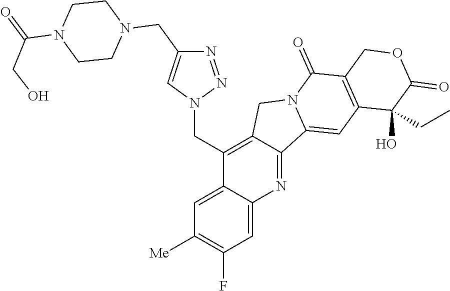 [SMILES: CC[C@@]1(O)C(=O)OCc2c1cc1n(c2=O)Cc2c-1nc1cc(F)c(C)cc1c2Cn1cc(CN2CCN(C(=O)CO)CC2)nn1] |
| 61 | 99% | 589.14 | 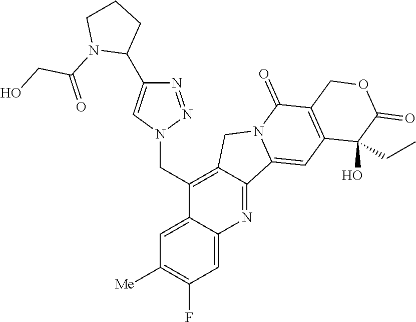 [SMILES: CC[C@@]1(O)C(=O)OCc2c1cc1n(c2=O)Cc2c-1nc1cc(F)c(C)cc1c2Cn1cc(C2CCCN2C(=O)CO)nn1] |
| 62 | 67% | 648.17 | 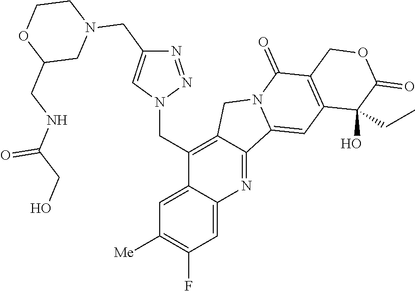 [SMILES: CC[C@@]1(O)C(=O)OCc2c1cc1n(c2=O)Cc2c-1nc1cc(F)c(C)cc1c2Cn1cc(CN2CCOC(CNC(=O)CO)C2)nn1] |
| 63 | 17% | 575.06 | 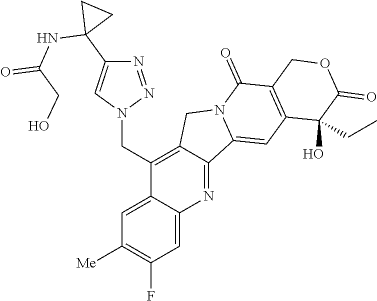 [SMILES: CC[C@@]1(O)C(=O)OCc2c1cc1n(c2=O)Cc2c-1nc1cc(F)c(C)cc1c2Cn1cc(C2(NC(=O)CO)CC2)nn1] |
| 64 | 34% | 563.17 | 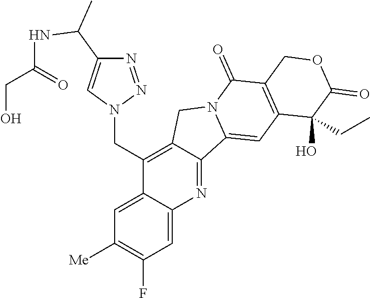 [SMILES: CC[C@@]1(O)C(=O)OCc2c1cc1n(c2=O)Cc2c-1nc1cc(F)c(C)cc1c2Cn1cc(C(C)NC(=O)CO)nn1] |
| 65 | 14% | 506.15 | 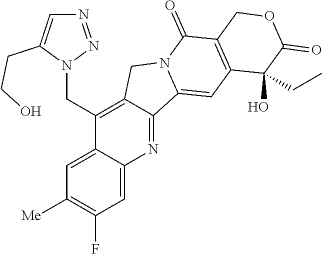 [SMILES: CC[C@@]1(O)C(=O)OCc2c1cc1n(c2=O)Cc2c-1nc1cc(F)c(C)cc1c2Cn1nncc1CCO] |
| 66 | 92% | 610.26 | 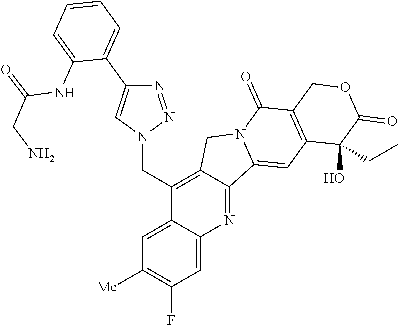 [SMILES: CC[C@@]1(O)C(=O)OCc2c1cc1n(c2=O)Cc2c-1nc1cc(F)c(C)cc1c2Cn1cc(-c2ccccc2NC(=O)CN)nn1] |
| 67 | 89% | 610.24 | 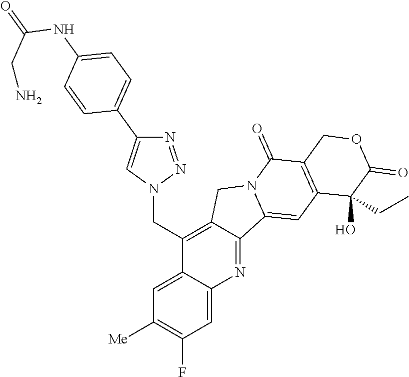 [SMILES: CC[C@@]1(O)C(=O)OCc2c1cc1n(c2=O)Cc2c-1nc1cc(F)c(C)cc1c2Cn1cc(-c2ccc(NC(=O)CN)cc2)nn1] |
| 68 | 32% | 577.26 | 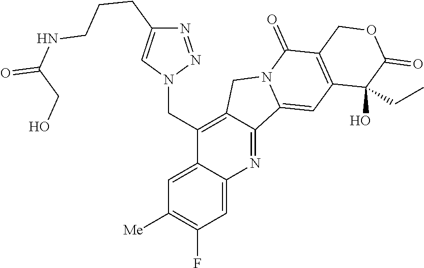 [SMILES: CC[C@@]1(O)C(=O)OCc2c1cc1n(c2=O)Cc2c-1nc1cc(F)c(C)cc1c2Cn1cc(CCCNC(=O)CO)nn1] |
| 69 | 21% | 577.11 | 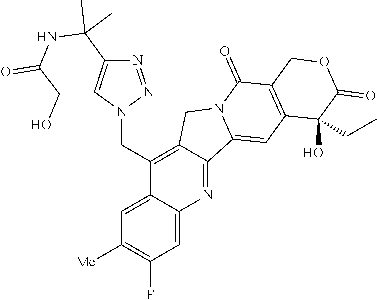 [SMILES: CC[C@@]1(O)C(=O)OCc2c1cc1n(c2=O)Cc2c-1nc1cc(F)c(C)cc1c2Cn1cc(C(C)(C)NC(=O)CO)nn1] |

### Example 05: Large Scale Synthesis of Compound 2

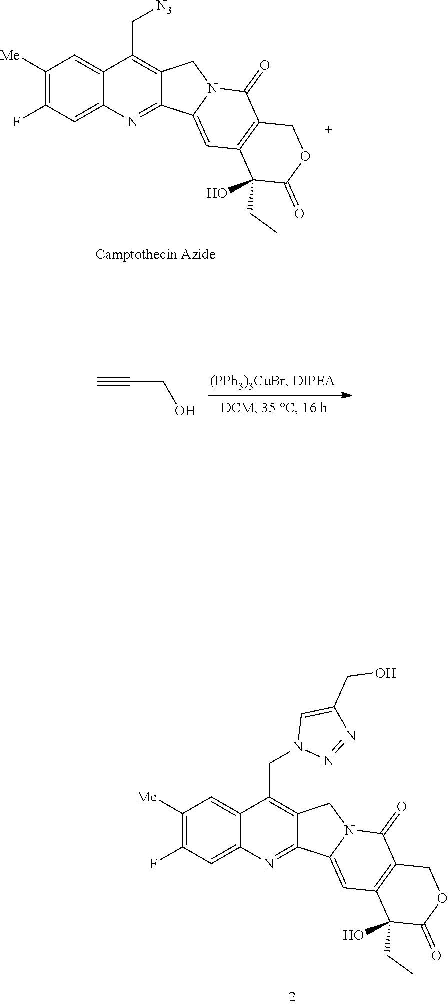

Camptothecin azide (0.25 g, 0.5742 mmol) and (0.132 ml, 2.297 mmol, 4 eq) propargyl alcohol were added to a flask under argon atmosphere. (PPh₃)₃CuBr (107 mg, 20 mol %) was added to the flask and dissolved in anhydrous dichloromethane (36 mL). Diisopropylethylamine (2.011 mL, 11.483 mmol, 20 eq) was added to the flask and the reaction was stirred at 35° C. for 16 hours sealed under argon atmosphere. The crude reaction was diluted with methanol (0.5 ml) and purified on 212 g silica (10% MeOH in DCM). The product was concentrated en vacuo to obtain compound 2 (0.154 g, 55% yield) as a light tan solid. (ESI) m/z: [M+H]+=491.06.

### Example 06: Large Scale Synthesis of Compound 5

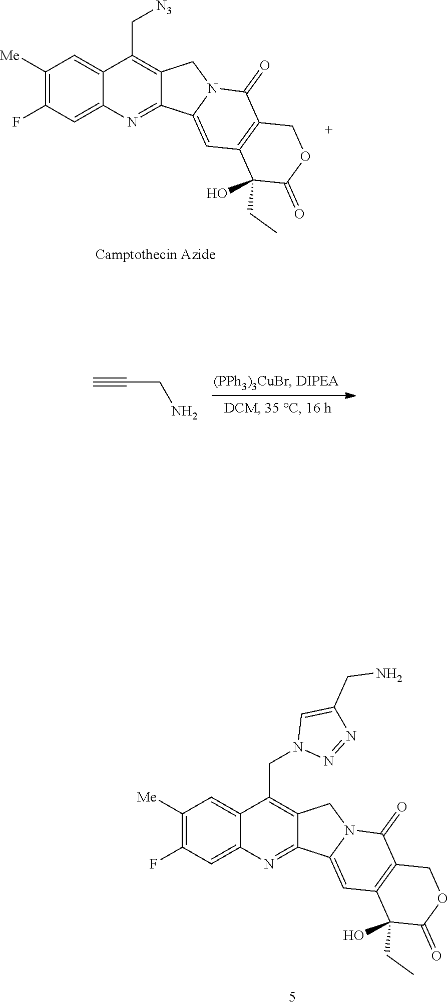

Camptothecin-azide (0.25 g, 0.5742 mmol) and (0.146 ml, 2.297 mmol, 4 eq) propargyl amine were added to a flask under argon atmosphere. (PPh₃)₃CuBr (107 mg, 20 mol %) was added to the flask and dissolved in anhydrous dichloromethane (36 mL). Diisopropylethylamine (2.011 mL, 11.483 mmol, 20 eq) was added to the flask and the reaction was stirred at 35° C. for 16 hours sealed under argon atmosphere. The crude reaction was concentrated en vacuo and dissolved in (50 mL) (2:1) (ACN:H₂O) (+0.0500 FA). The crude material was purified via reverse phase chromatography (275 g C18) (500 ACN+0.050% FA)/(H₂O+0.0500 FA) to (9500 ACN+0.05% FA)/(H₂O+0.0500 FA) gradient). The product was recovered by lyophilization to obtain compound 5 (0.187 g, 670% yield) as a light tan solid. (ESI) m/z: [M+H]+=491.17.

TABLE 02
Linkers Proposed to be attached to Payloads (“PL”) compounds 1-69.
Several linkers contain amino acid sequence GGFG (SEQ ID NO: 1).

| Linker | Payload | |
|---|---|---|
| Abbreviation | Type | Structure |
| MC | Amino- PL | 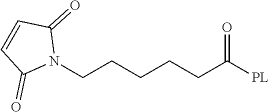 |
| PEG MC | Amino- PL | 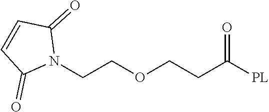 |
| Aryl MC | Amino- PL | 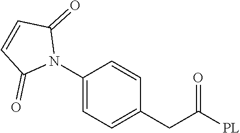 |
| MC-G | Amino- PL | 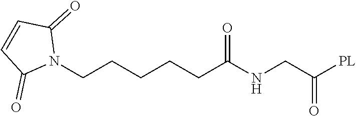 |
| MC-A | Amino- PL | 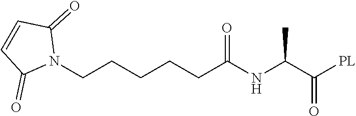 |
| MC-A-A | Amino- PL | 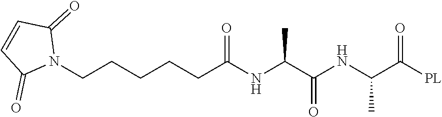 |
| MC-A-A-A | Amino- PL | 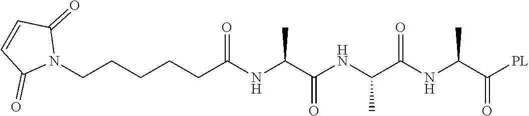 |
| MC-G-G-F-G | Amino- PL | 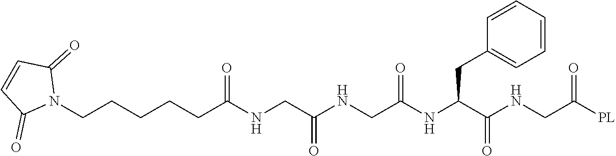 |
| MC-G-G-F- G- hemiaminal | Alcohol- PL | 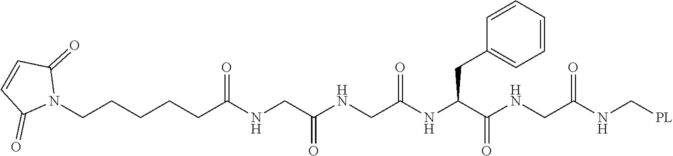 |
| MC-G-G-F- G- hemiaminal- glycolic acid | Amine- PL | 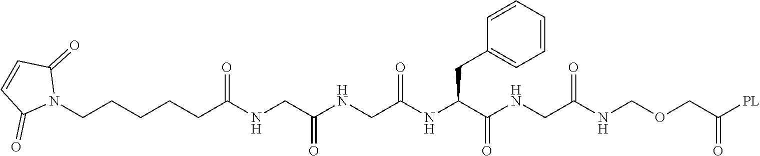 |
| MC-V-A | Amino- PL | 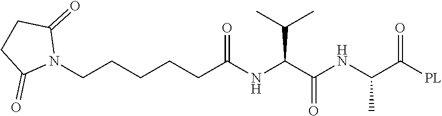 |
| MC-V-C | Amino- PL | 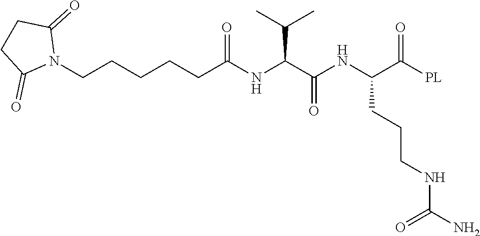 |
| MC-V-C- PAB | Amino- PL | 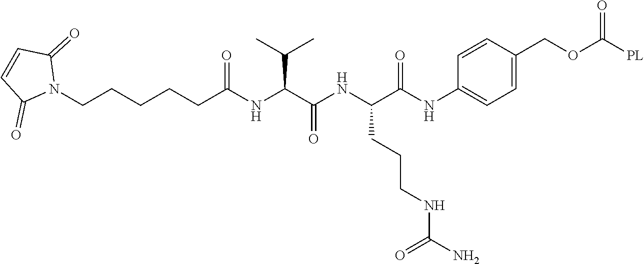 |
| MC-E-V-C | Amino- PL | 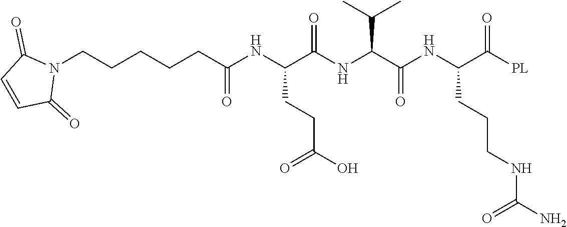 |
| MC-E-V-C- PAB | Amino- PL | 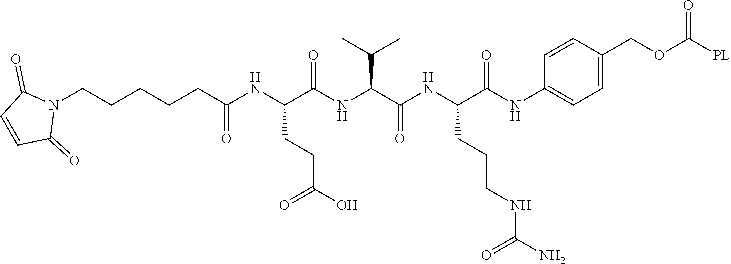 |
| PEG MC-G | Amino- PL | 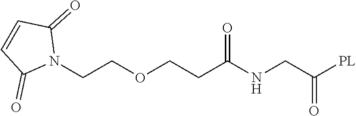 |
| PEG MC-A | Amino- PL | 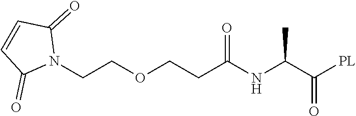 |
| PEG MC-A- A | Amino- PL | 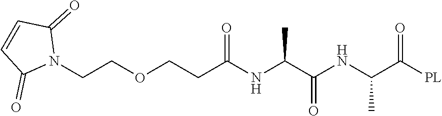 |
| PEG MC-A- A-A | Amino- PL | 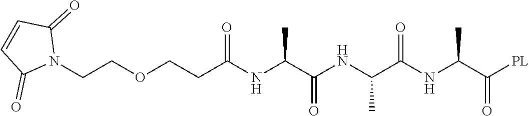 |
| PEG MC-G- G-F-G | Amino- PL | 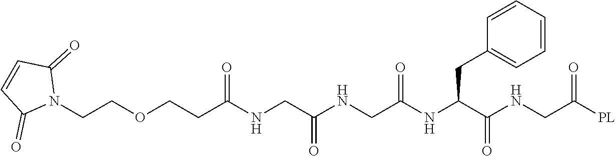 |
| PEG MC-G- G-F-G- hemiaminal | Alcohol- PL | 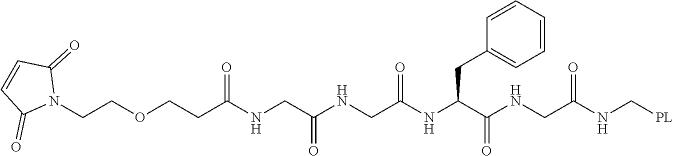 |
| PEG MC-G- G-F-G- hemiaminal- glycolic acid | Amine- PL | 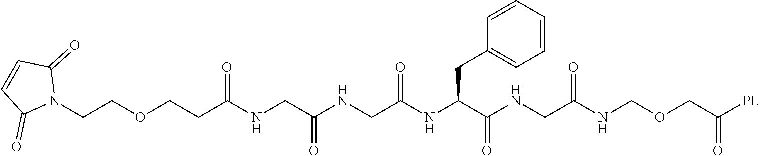 |
| PEG MC-V- A | Amino- PL | 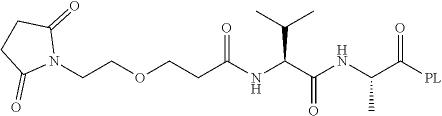 |
| PEG MC-V- C | Amino- PL | 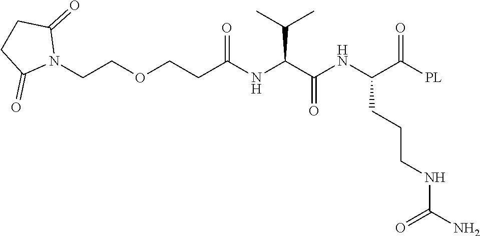 |
| PEG MC-V- C-PAB | Amino- PL | 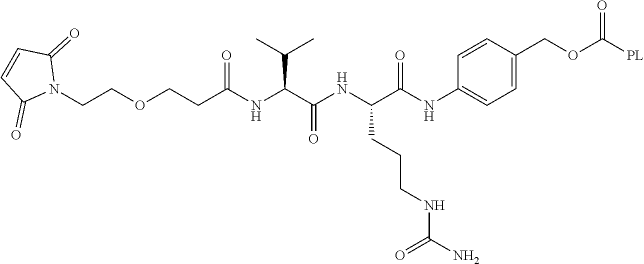 |
| PEG MC-E- V-C | Amino- PL | 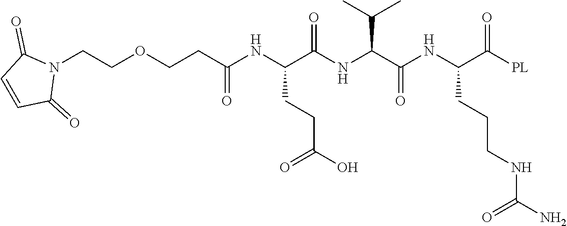 |
| PEG MC-E- V-C-PAB | Amino- PL | 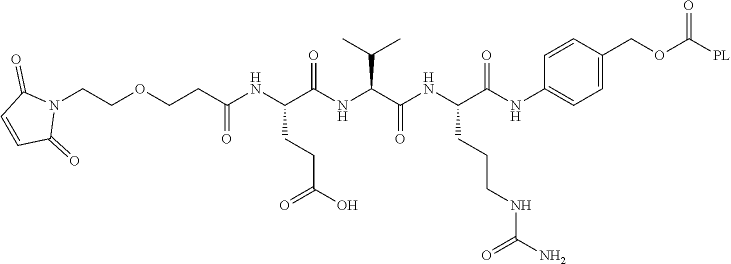 |
| Aryl MC-G | Amino- PL | 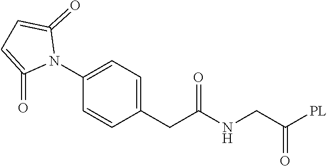 |
| Aryl MC-A | Amino- PL | 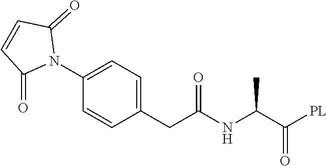 |
| Aryl MC-A- A | Amino- PL | 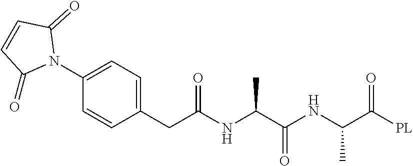 |
| Aryl MC-A- A-A | Amino- PL | 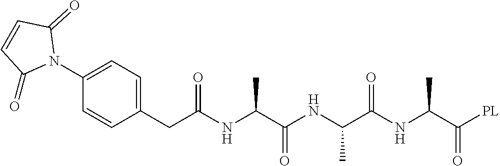 |
| Aryl MC-G- G-F-G | Amino- PL | 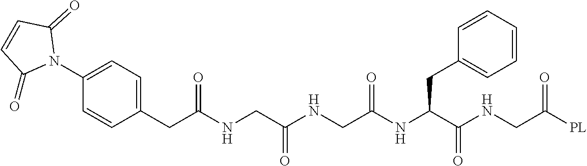 |
| Aryl MC-G- G-F-G- hemiaminal | Alcohol- PL | 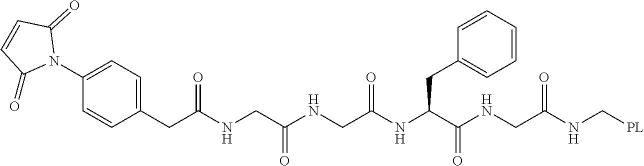 |
| Aryl MC-G- G-F-G- hemiaminal- glycolic acid | Amine- PL | 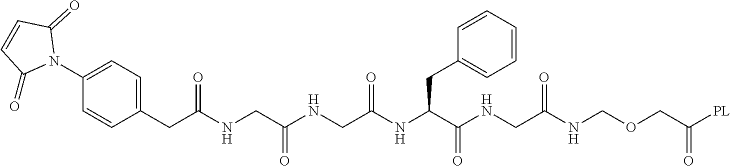 |
| Aryl MC-V- A | Amino- PL | 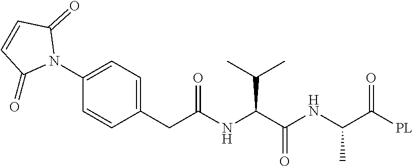 |
| Aryl MC-V- C | Amino- PL | 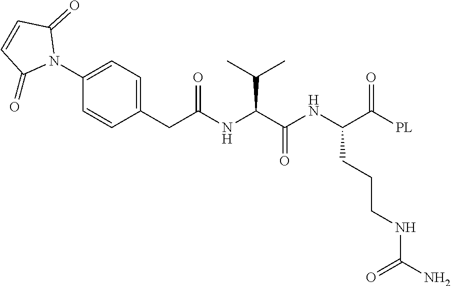 |
| Aryl MC-V- C-PAB | Amino- PL | 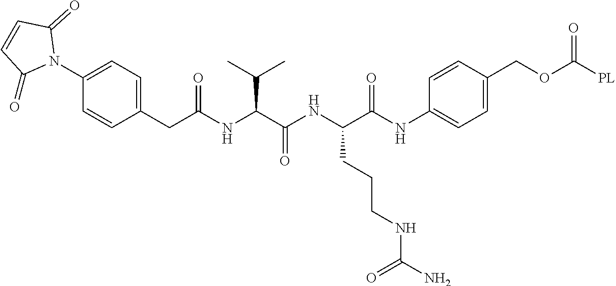 |
| Aryl MC-E- V-C | Amino- PL | 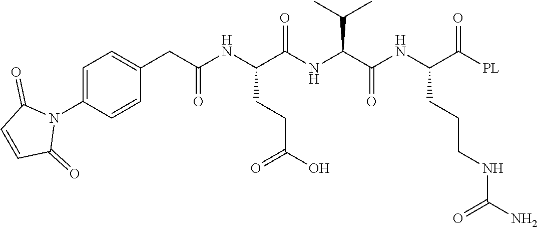 |
| Aryl MC-E- V-C-PAB | Amino- PL | 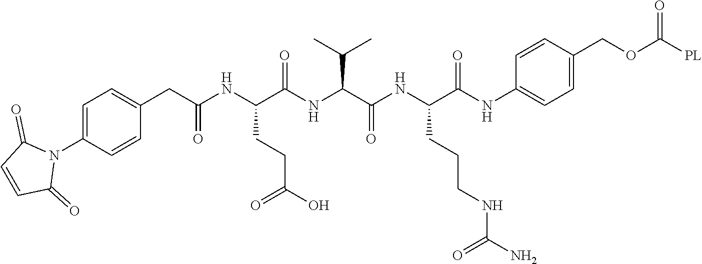 |
| Thiol- | Amino- | Disulfide linkers with terminal maleimide as described in Chem. |
| Disulfides | PL | Soc. Rev., 2019,48, 4361-4374, incorporated by reference herein |

### Example 07: Synthesis of Payload Linkers

### Example 08: Synthesis of PL1

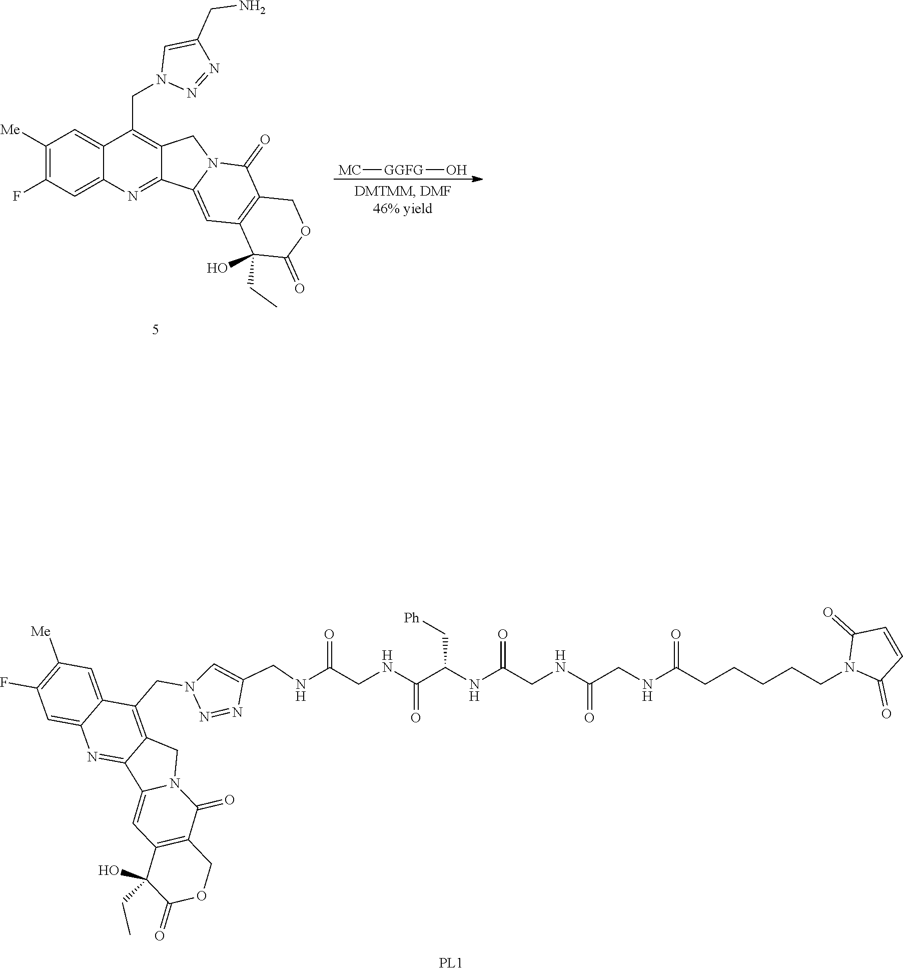

To an oven dried vial was added 5 (27 mg, 0.054 mmol, 1 eq) and MC-GGFG-OH (36 mg, 0.068 mmol, 1.25 eq) under Ar. Anhydrous DMF (1.54 mL) was added to the vial to yield a solution. DMTMM (30 mg, 0.108 mmol, 2 eq) was added to the vial and briefly sonicated to yield a suspension. The vial was stirred at room temperature for 2 h whereupon the LCMS indicated 5 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL1 (25.2 mg, 46% yield) as a white solid. (ESI) m/z: [M+H]+=1002.12

### Example 09: Synthesis of PL2

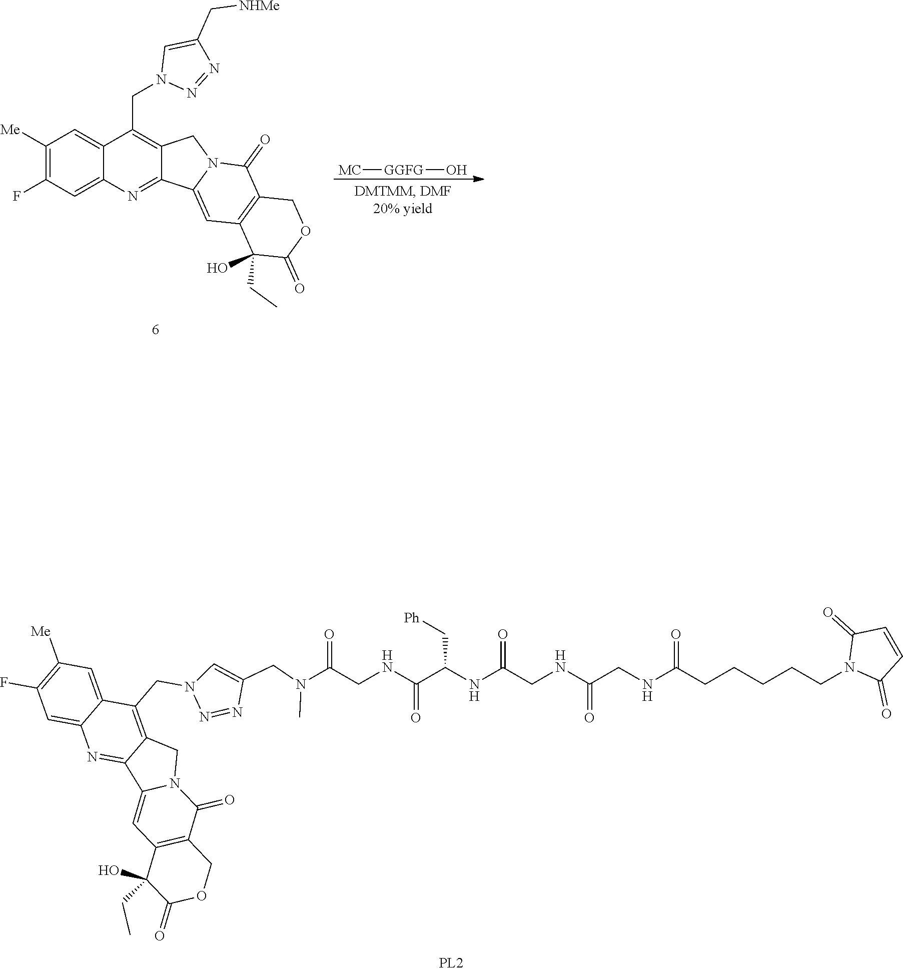

To an oven dried vial was added 6 (2.0 mg, 0.004 mmol, 1 eq) and MC-GGFG-OH (2.9 mg, 0.005 mmol, 1.25 eq) under Ar. Anhydrous DMF (0.3 mL) was added to the vial to yield a solution. DMTMM (2.4 mg, 0.009 mmol, 2 eq) was added to the vial and briefly sonicated to yield a suspension. The vial was stirred at room temperature for 0.5 h whereupon the LCMS indicated 6 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL2 (0.8 mg, 20% yield) as a white solid. (ESI) m/z: [M+H]+=1016.32

### Example 10: Synthesis of PL3

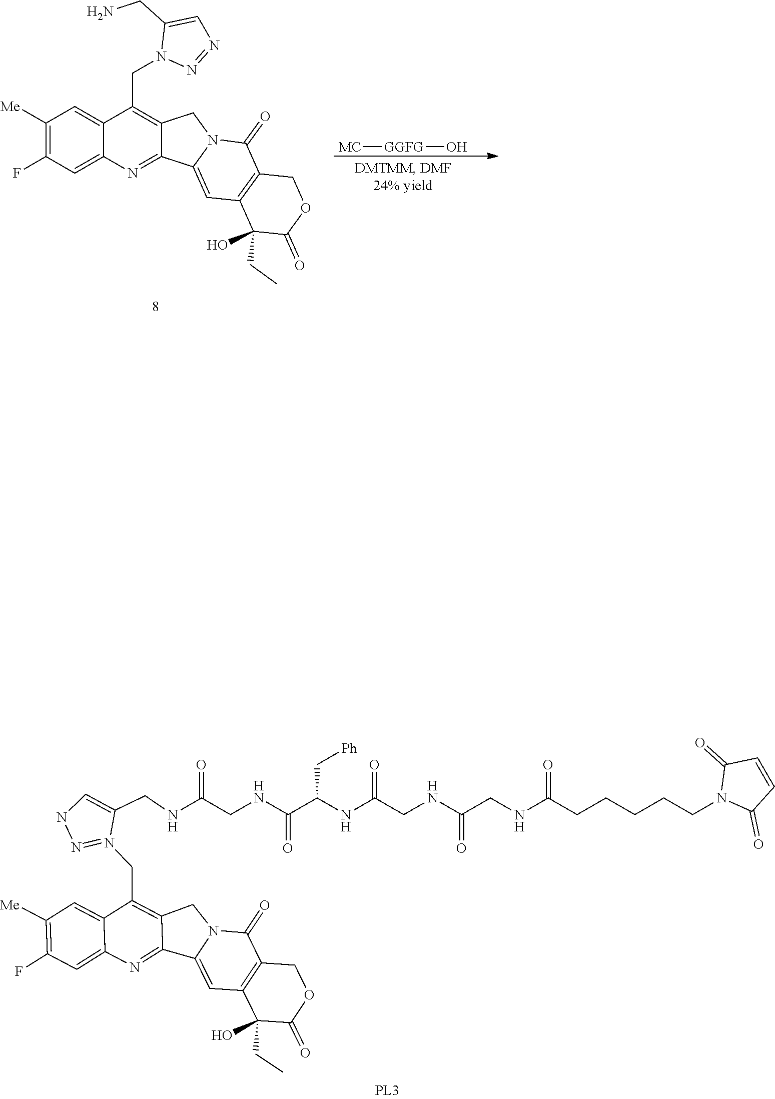

To an oven dried vial was added 8 (4.0 mg, 0.008 mmol, 1 eq) and MC-GGFG-OH (5.5 mg, 0.010 mmol, 1.25 eq) under Ar. Anhydrous DMF (0.15 mL) was added to the vial to yield a solution. DMTMM (4.6 mg, 0.017 mmol, 2 eq) was added to the vial and briefly sonicated to yield a suspension. The vial was stirred at room temperature for 1 h whereupon the LCMS indicated 8 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL3 (2.0 mg, 24% yield) as a white solid. (ESI) m/z: [M+H]+=1001.72

### Example 11: Synthesis of PL4

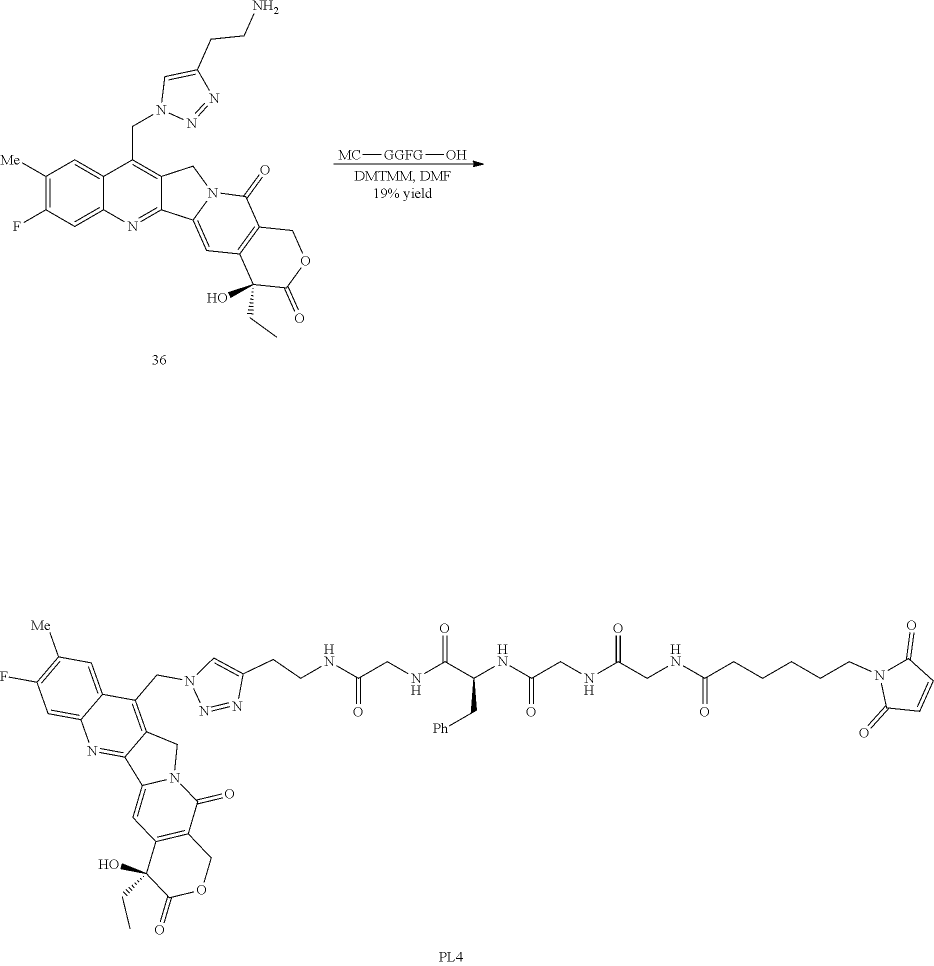

To an oven dried vial was added 36 (13.0 mg, 0.026 mmol, 1 eq) and MC-GGFG-OH (17.3 mg, 0.033 mmol, 1.25 eq) under Ar. Anhydrous DMF (0.47 mL) was added to the vial to yield a solution. DMTMM (14.5 mg, 0.052 mmol, 2 eq) was added to the vial and briefly sonicated to yield a suspension. The vial was stirred at room temperature for 2 h whereupon the LCMS indicated 36 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL4 (5.1 mg, 19% yield) as a white solid. (ESI) m/z: [M+H]+=1016.82.

### Example 12: Synthesis of PL5

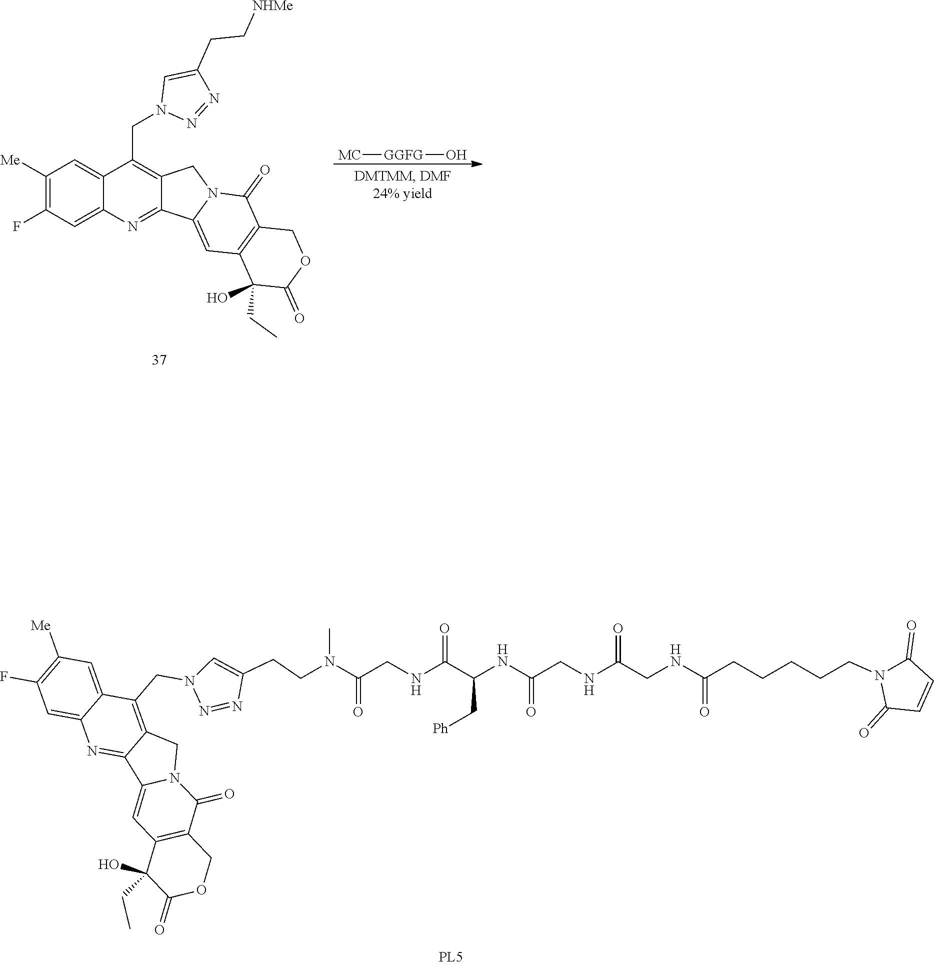

To an oven dried vial was added 37 (12.0 mg, 0.024 mmol, 1 eq) and MC-GGFG-OH (15.8 mg, 0.030 mmol, 1.25 eq) under Ar. Anhydrous DMF (0.43 mL) was added to the vial to yield a solution. DMTMM (13.2 mg, 0.048 mmol, 2 eq) was added to the vial and briefly sonicated to yield a suspension. The vial was stirred at room temperature for 0.5 h whereupon the LCMS indicated 37 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL5 (6.0 mg, 24% yield) as a white solid. (ESI) m/z: [M+H]+=1030.56.

### Example 13: Synthesis of PL6

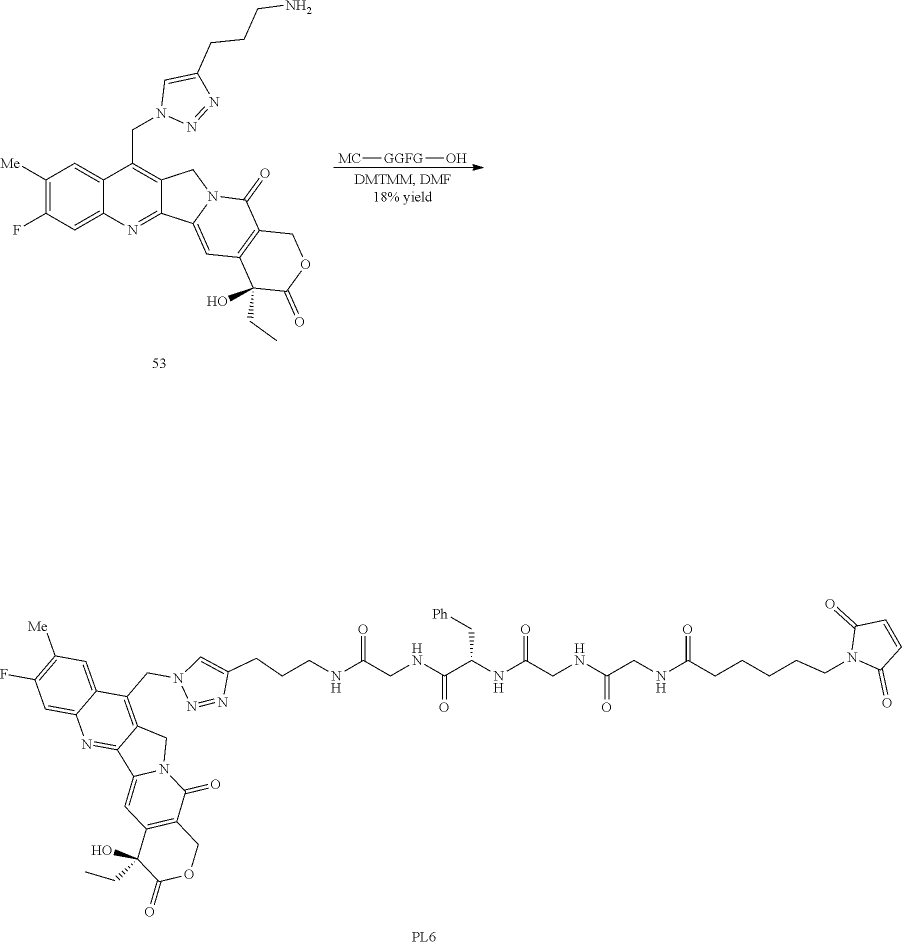

To an oven dried vial was added 53 (9.0 mg, 0.016 mmol, 1 eq) and MC-GGFG-OH (10.9 mg, 0.020 mmol, 1.25 eq) under Ar. Anhydrous DMF (0.68 mL) was added to the vial to yield a solution. DMTMM (9.1 mg, 0.033 mmol, 2 eq) was added to the vial and briefly sonicated to yield a suspension. The vial was stirred at room temperature for 0.5 h whereupon the LCMS indicated 53 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL6 (3.0 mg, 18% yield) as a white solid. (ESI) m/z: [M+H]+=1030.60.

### Example 14: Synthesis of PL7

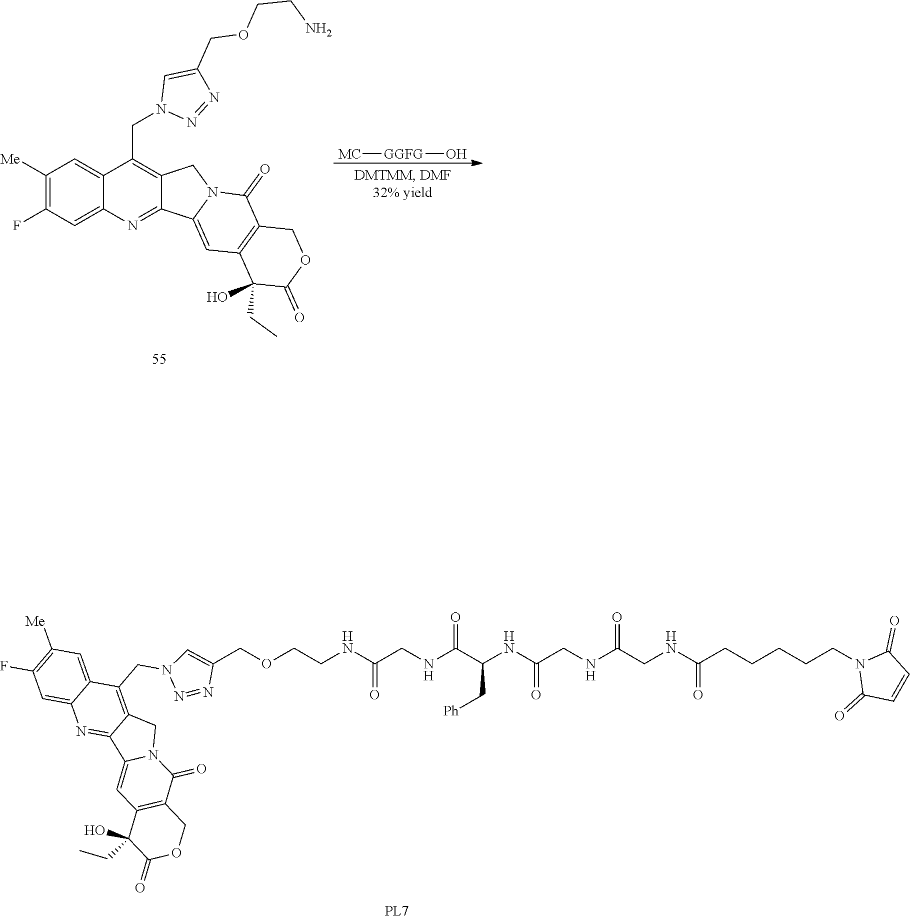

To an oven dried vial was added 55 (9.0 mg, 0.017 mmol, 1 eq) and MC-GGFG-OH (11.1 mg, 0.021 mmol, 1.25 eq) under Ar. Anhydrous DMF (0.70 mL) was added to the vial to yield a solution. DMTMM (9.3 mg, 0.034 mmol, 2 eq) was added to the vial and briefly sonicated to yield a suspension. The vial was stirred at room temperature for 0.5 h whereupon the LCMS indicated 55 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL7 (5.6 mg, 31.8% yield) as a white solid. (ESI) m/z: [M+H]+=1046.75.

### Example 15: Synthesis of PL8

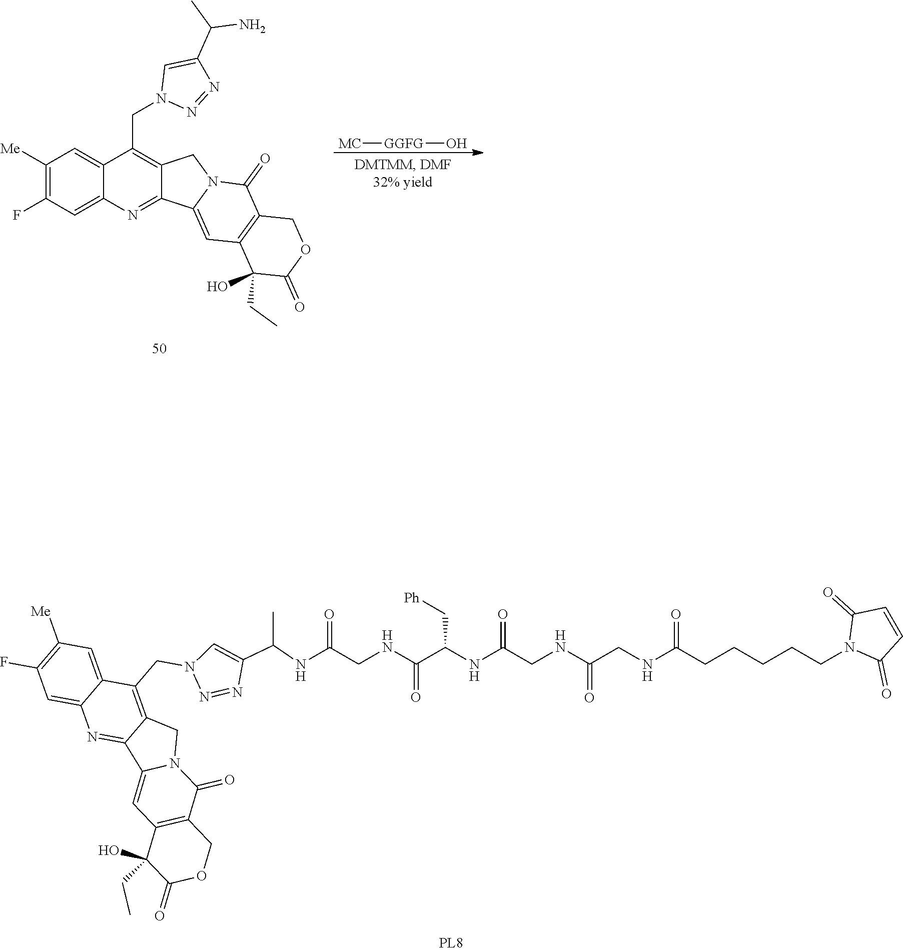

To an oven dried vial was added 50 (10.0 mg, 0.019 mmol, 1 eq) and MC-GGFG-OH (12.9 mg, 0.024 mmol, 1.25 eq) under Ar. Anhydrous DMF (0.81 mL) was added to the vial to yield a solution. DMTMM (10.8 mg, 0.039 mmol, 2 eq) was added to the vial and briefly sonicated to yield a suspension. The vial was stirred at room temperature for 0.5 h whereupon the LCMS indicated 50 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL8 (6.3 mg, 32% yield) as a white solid. (ESI) m/z: [M+H]+=1016.35.

### Example 16: Synthesis of PL9

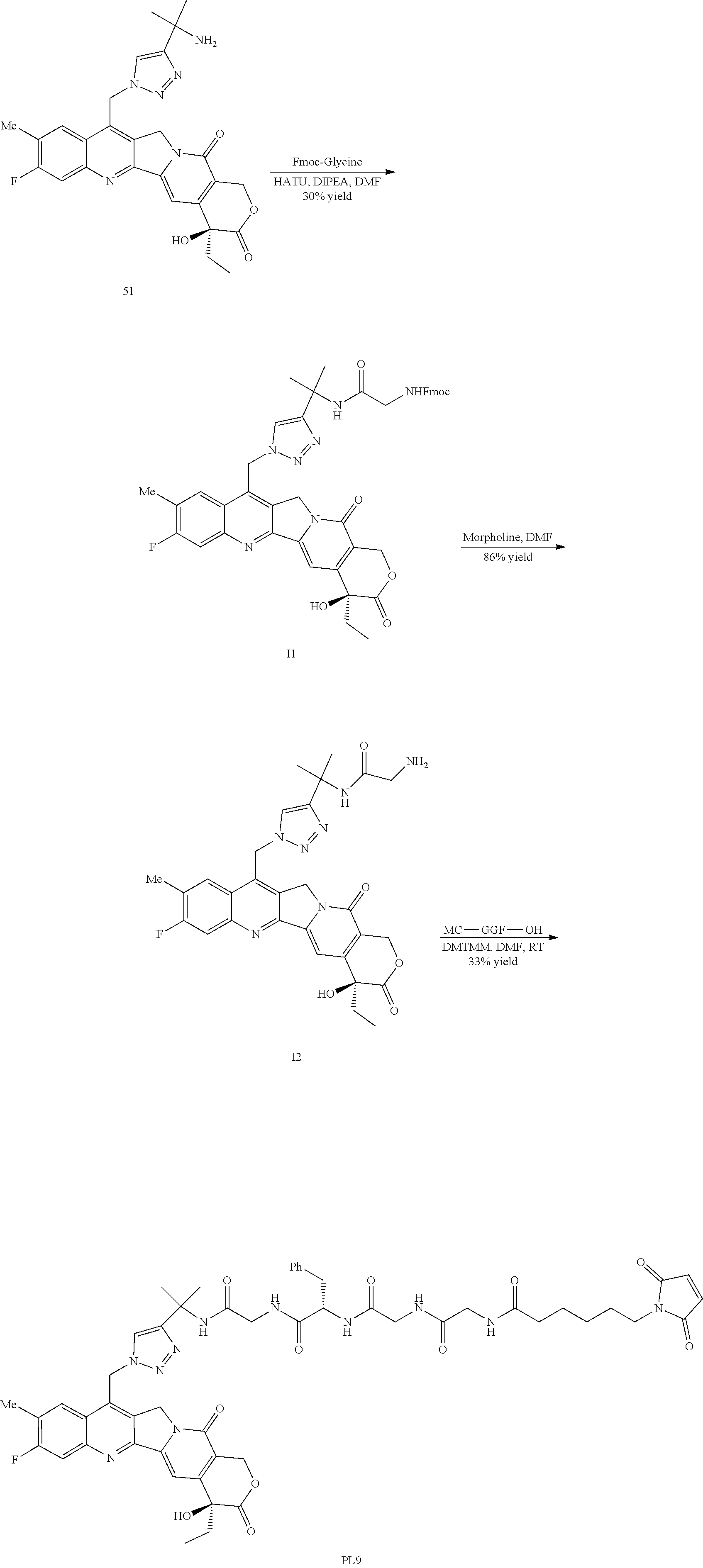

To an oven dried vial was added Fmoc-glycine (25.1 mg, 0.084 mmol, 2 eq) with Anhydrous DMF (1.21 mL). To the vial was added DIPEA (29.4 uL, 0.169 mmol, 4 eq) followed by HATU (32.1 mg, 0.084 mmol, 2 eq) and stirred 10 minutes at room temperature. After ten minutes, 51 (22.2 mg, 0.042 mmol, 1 eq) was added to the vial and stirred at room temperature for 2.5 h whereupon LCMS indicated 51 was consumed. The crude reaction was purified via reverse phase (Teledyne Gemini (5 μm C18 110 Å 150×30 mm) with (5% ACN+0.05% FA)/(H₂O+0.05% FA) to 95% (ACN+0.05% FA)/(H₂O+0.05% FA) gradient). The product was recovered by lyophilization to obtain I1 (10.9 mg, 30% yield) as a white solid. (ESI) m/z: [M+H]+=798.58.

To an oven dried vial was added I1 (11 mg, 0.014 mmol, 1 eq) and dissolved in (1:4) (Morpholine:DMF) (0.195 mL) and stirred at room temperature under Ar. LCMS at 90 minutes indicated that I1 was consumed. The crude reaction was precipitated with diethyl ether (2 mL) and the solid was obtained by centrifugation. The solid was washed with diethyl ether (1×) (2 mL) and the solid was obtained by centrifugation. This obtained solid was redissolved in (1:1) (H₂O:ACN) and lyophilized to give 12 (6.8 mg, 86% yield) as a white solid. (ESI) m/z: [M+H]+=576.20.

To an oven dried vial was added I2 (7.0 mg, 0.012 mmol, 1 eq) and MC-GGF-OH (7.0 mg, 0.015 mmol, 1.25 eq) under Ar. Anhydrous DMF (0.37 mL) was added to the vial to yield a solution. DMTMM (6.5 mg, 0.024 mmol, 2 eq) was added to the vial and briefly sonicated to yield a suspension. The vial was stirred at room temperature for 0.5 h whereupon the LCMS indicated I2 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL9 (4.0 mg, 33% yield) as a white solid. (ESI) m/z: [M+H]+=1030.60.

### Example 17: Synthesis of PL10

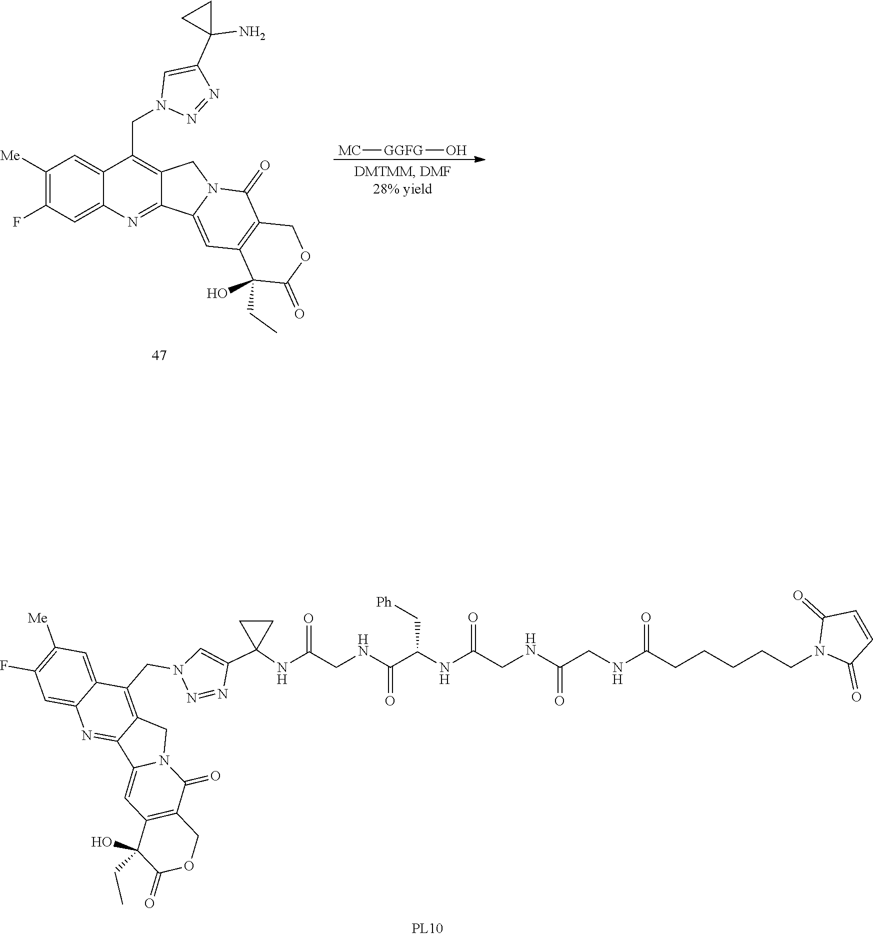

To an oven dried vial was added 47 (18.0 mg, 0.035 mmol, 1 eq) and MC-GGFG-OH (23.5 mg, 0.044 mmol, 1.25 eq) under Ar. Anhydrous DMF (0.1.11 mL) was added to the vial to yield a solution. NMM (5 uL, 0.045 mmol, 1.25 eq) was added to the vial followed by DMTMM (19.6 mg, 0.071 mmol, 2 eq) was added to the vial and briefly sonicated to yield a suspension. The vial was stirred at room temperature for 0.25 h whereupon the LCMS indicated 47 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL10 (10.0 mg, 27.5% yield) as a white solid. (ESI) m/z: [M+H]+=1028.34.

### Example 18: Synthesis of PL11

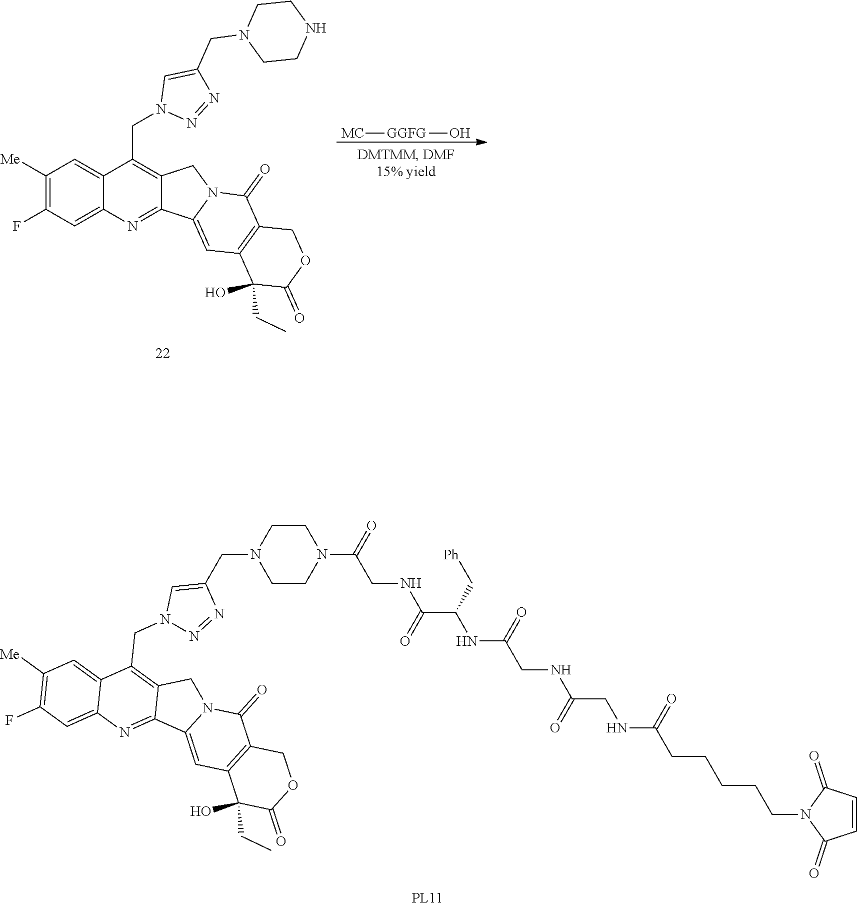

To an oven dried vial was added 22 (12.0 mg, 0.021 mmol, 1 eq) and MC-GGFG-OH (14.2 mg, 0.027 mmol, 1.25 eq) under Ar. Anhydrous DMF (0.67 mL) was added to the vial to yield a solution. DMTMM (11.9 mg, 0.043 mmol, 2 eq) was added to the vial and briefly sonicated to yield a suspension. The vial was stirred at room temperature for 2 h whereupon the LCMS indicated 22 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL11 (3.4 mg, 15% yield) as a white solid. (ESI) m/z: [M+H]+=1071.46.

### Example 19: Synthesis of PL12

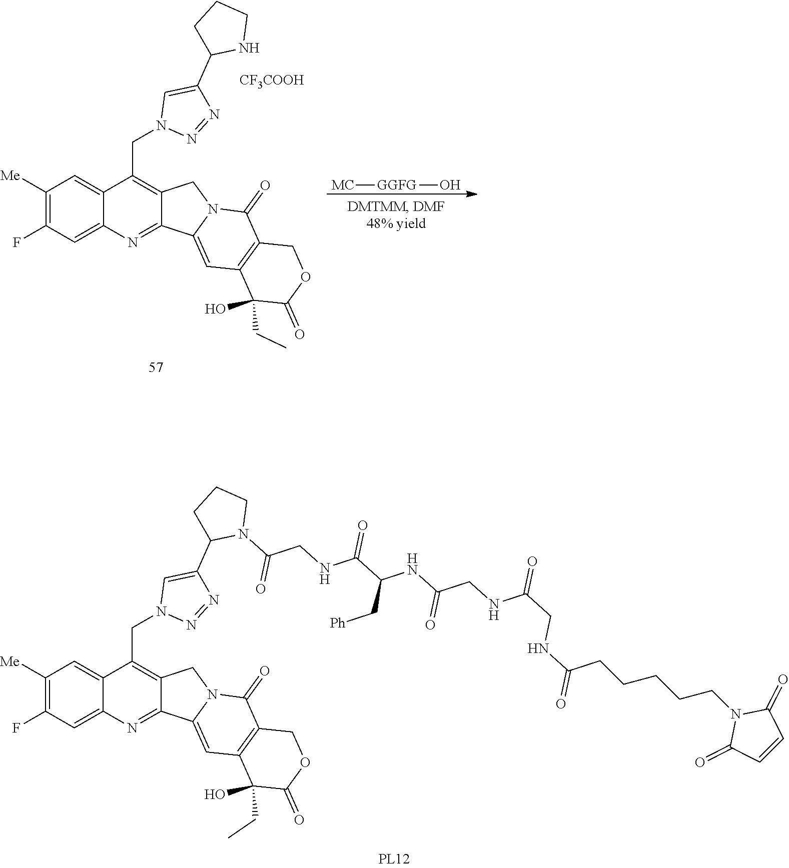

To an oven dried vial was added 57 (10.0 mg, 0.016 mmol, 1 eq) and MC-GGFG-OH (10.5 mg, 0.020 mmol, 1.25 eq) under Ar. Anhydrous DMF (0.66 mL) was added to the vial to yield a solution. NMM (2 uL, 0.02 mmol, 1.25 eq) was added to the vial followed by DMTMM (8.8 mg, 0.032 mmol, 2 eq) and briefly sonicated to yield a suspension. The vial was stirred at room temperature for 0.5 h whereupon the LCMS indicated 57 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL12 (7.9 mg, 48% yield) as a white solid. (ESI) m/z: [M+H]+=1042.08.

### Example 20: Synthesis of PL13

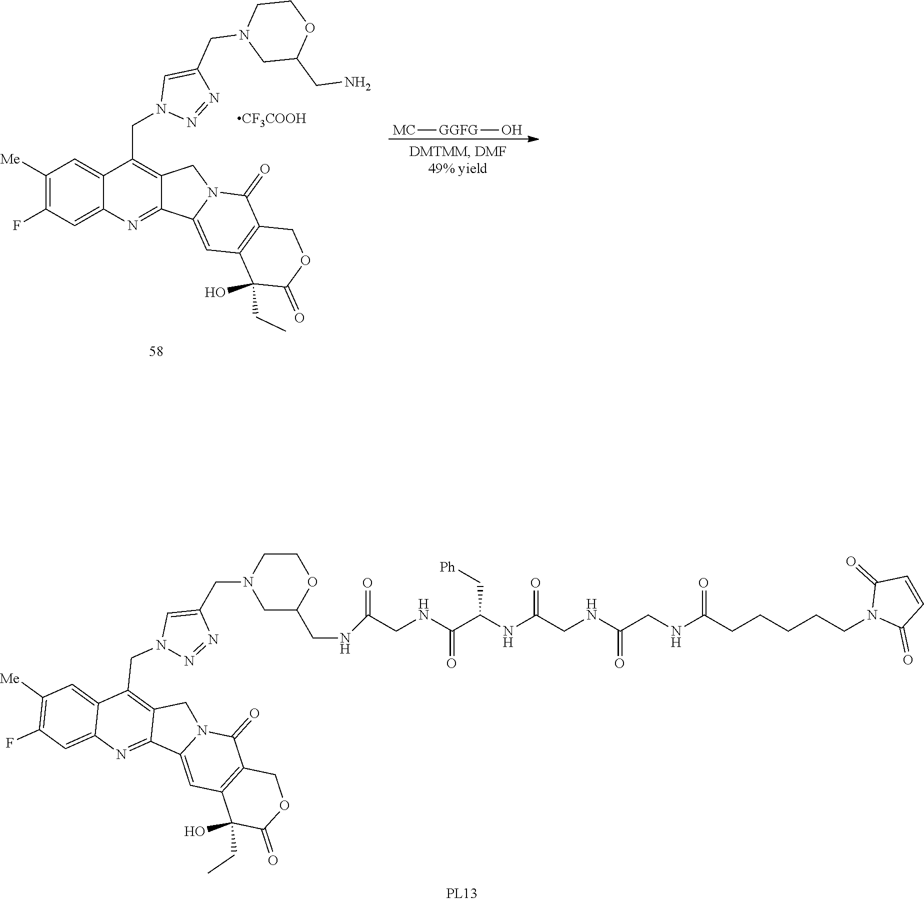

To an oven dried vial was added 58 (15.0 mg, 0.021 mmol, 1 eq) and MC-GGFG-OH (14.1 mg, 0.027 mmol, 1.25 eq) under Ar. Anhydrous DMF (0.89 mL) was added to the vial to yield a solution. NMM (3 uL, 0.027 mmol, 1.25 eq) was added to the vial followed by DMTMM (14.5 mg, 0.052 mmol, 2 eq) and briefly sonicated to yield a suspension. The vial was stirred at room temperature for 0.5 h whereupon the LCMS indicated 58 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL13 (11.5 mg, 49% yield) as a white solid. (ESI) m/z: [M+H]+=1101.92.

### Example 21: Synthesis of PL14

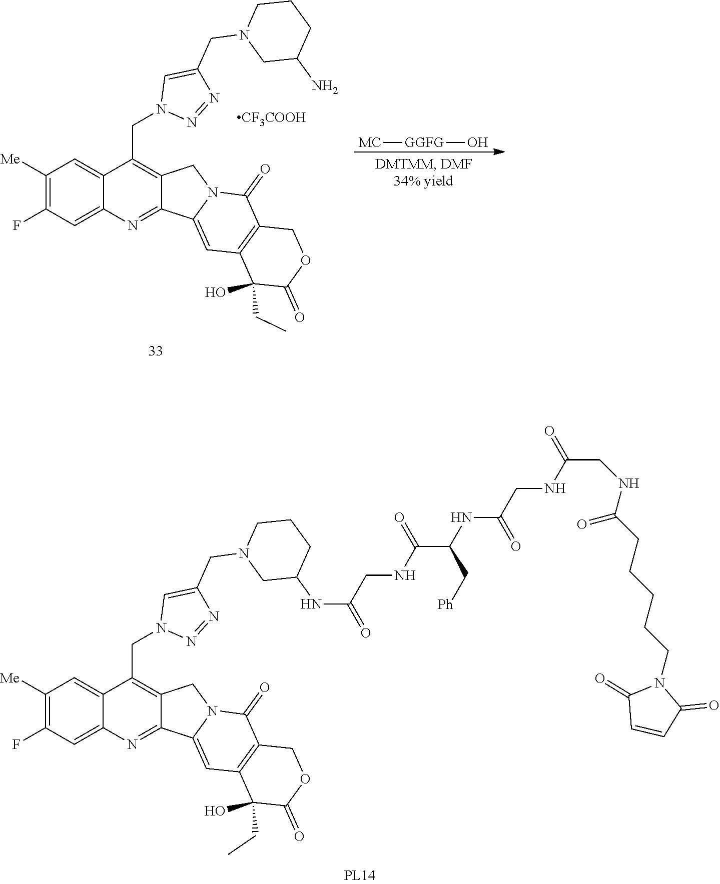

To an oven dried vial was added 33 (11.0 mg, 0.016 mmol, 1 eq) and MC-GGFG-OH (10.4 mg, 0.020 mmol, 1.25 eq) under Ar. Anhydrous DMF (0.49 mL) was added to the vial to yield a solution. NMM (2 uL, 0.02 mmol, 1.25 eq) was added to the vial followed by DMTMM (14.5 mg, 0.052 mmol, 2 eq) and briefly sonicated to yield a suspension. The vial was stirred at room temperature for 0.5 h whereupon the LCMS indicated 33 was consumed. The crude reaction was purified via reverse (Teledyne Gemini (5 μm C18 110 Å 150×30 mm) with (5% ACN)/(H₂O) to 95% (ACN)/(H₂O) gradient). The product was recovered by lyophilization to obtain PL14 (5.8 mg, 34% yield) as a white solid. (ESI) m/z: [M+H]+=1085.51.

### Example 22: Synthesis of PL15

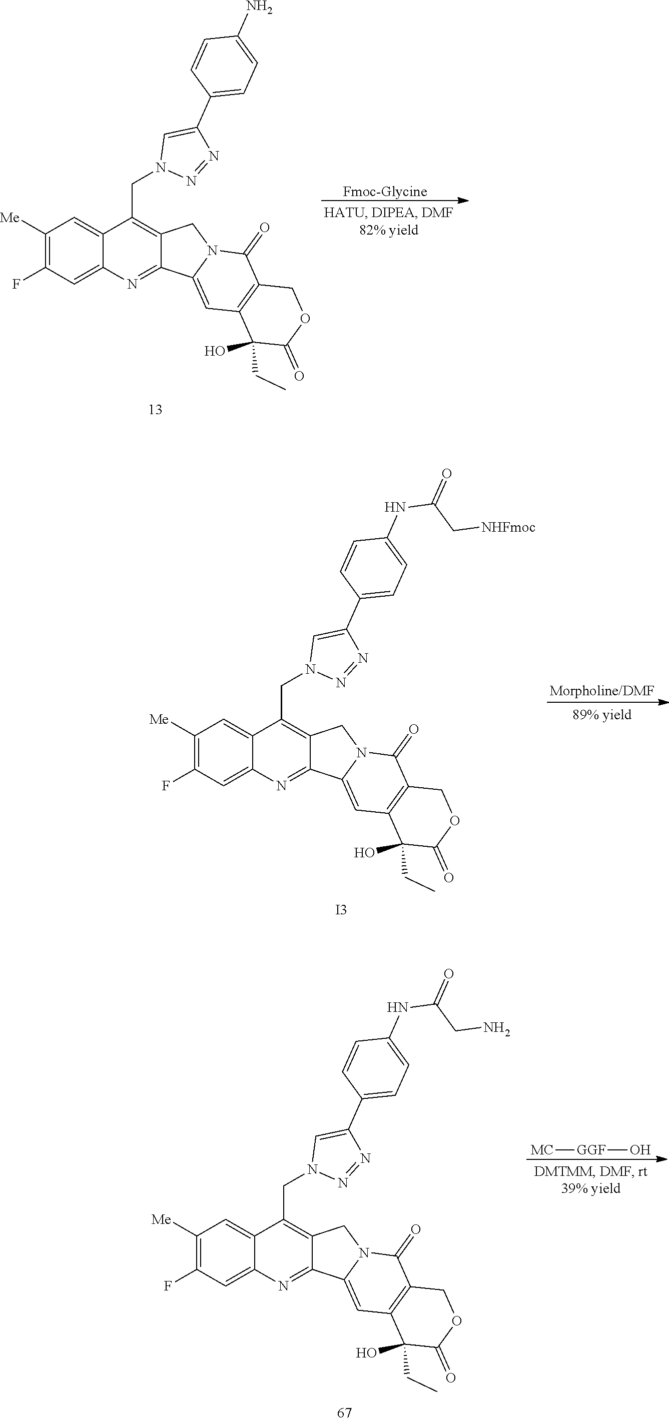

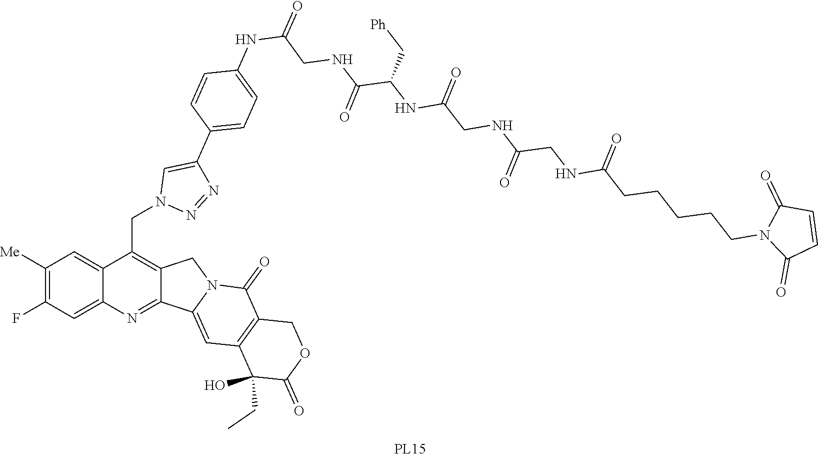 [SMILES: CC[C@@]1(O)C(=O)OCc2c1cc1n(c2=O)Cc2c-1nc1cc(F)c(C)cc1c2Cn1cc(-c2ccc(NC(=O)CNC(=O)[C@H](Cc3ccccc3)NC(=O)CNC(=O)CNC(=O)CCCCCN3C(=O)C=CC3=O)cc2)nn1]

To an oven dried vial was added Fmoc-glycine (11.9 mg, 0.040 mmol, 2 eq) with Anhydrous DMF (0.57 mL). To the vial was added DIPEA (14.0 uL, 0.080 mmol, 4 eq) followed by HATU (15.3 mg, 0.040 mmol, 2 eq) and stirred 10 minutes at room temperature. After ten minutes, 13 (11.0 mg, 0.020 mmol, 1 eq) was added to the vial and stirred at room temperature for 16 h whereupon LCMS indicated I3 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain 13 (13.7 mg, 82% yield) as a white solid. (ESI) m/z: [M+H]+=832.06.

To an oven dried vial was added I3 (14 mg, 0.016 mmol, 1 eq) and dissolved in (1:4) (Morpholine:DMF) (0.235 mL) and stirred at room temperature under Ar. LCMS at 60 minutes indicated that 13 was consumed. The crude reaction was precipitated with diethyl ether (2.5 mL) and the solid was obtained by centrifugation. The solid was washed with diethyl ether (1×) (2.5 mL) and the solid was obtained by centrifugation. This obtained solid was redissolved in (1:1) (H₂O:ACN) and lyophilized to give 67 (8.9 mg, 89% yield) as a white solid. (ESI) m/z: [M+H]+=610.24.

To an oven dried vial was added 67 (8.0 mg, 0.013 mmol, 1 eq) and MC-GGF-OH (7.5 mg, 0.016 mmol, 1.25 eq) under Ar. Anhydrous DMF (0.23 mL) was added to the vial to yield a solution. DMTMM (7.0 mg, 0.025 mmol, 2 eq) was added to the vial and briefly sonicated to yield a suspension. The vial was stirred at room temperature for 0.5 h whereupon the LCMS indicated 67 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL15 (5.2 mg, 39% yield) as a white solid. (ESI) m/z: [M+H]+=1064.26.

### Example 23: Synthesis of PL16

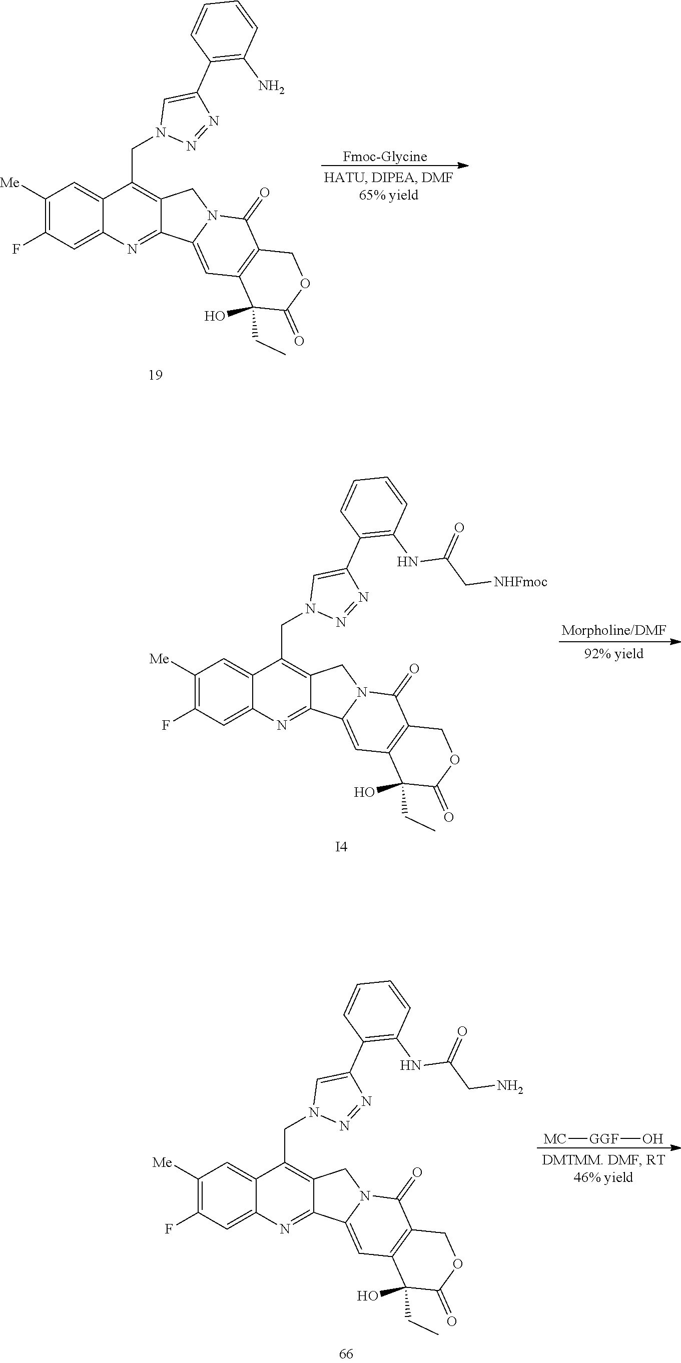

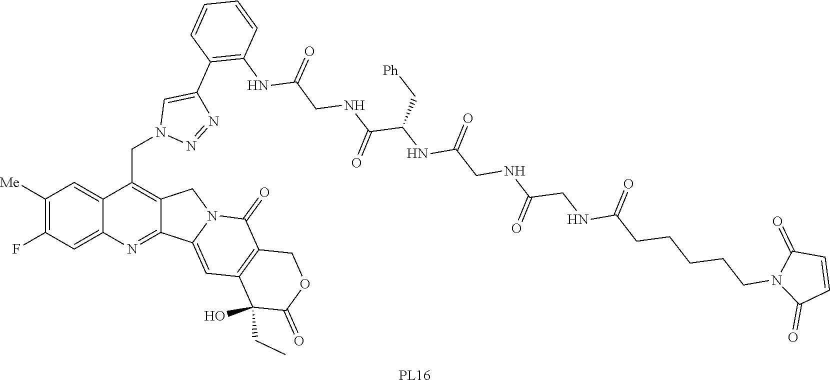 [SMILES: CC[C@@]1(O)C(=O)OCc2c1cc1n(c2=O)Cc2c-1nc1cc(F)c(C)cc1c2Cn1cc(-c2ccccc2NC(=O)CNC(=O)[C@H](Cc2ccccc2)NC(=O)CNC(=O)CNC(=O)CCCCCN2C(=O)C=CC2=O)nn1]

To an oven dried vial was added Fmoc-glycine (17.0 mg, 0.057 mmol, 2 eq) with Anhydrous DMF (0.82 mL). To the vial was added DIPEA (20.0 uL, 0.114 mmol, 4 eq) followed by HATU (22 mg, 0.057 mmol, 2 eq) and stirred 10 minutes at room temperature. After ten minutes, 19 (16.0 mg, 0.029 mmol, 1 eq) was added to the vial and stirred at room temperature for 4 h whereupon LCMS indicated I9 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain 14 (21.4 mg, 65% yield) as a white solid. (ESI) m/z: [M+H]+=832.23.

To an oven dried vial was added I4 (21 mg, 0.026 mmol, 1 eq) and dissolved in (1:4) (Morpholine:DMF) (0.368 mL) and stirred at room temperature under Ar. LCMS at 60 minutes indicated that 14 was consumed. The crude reaction was precipitated with diethyl ether (3.7 mL) and the solid was obtained by centrifugation. The solid was washed with diethyl ether (1×) (3.7 mL) and the solid was obtained by centrifugation. This obtained solid was redissolved in (1:1) (H₂O:ACN) and lyophilized to give 66 (14.5 mg, 92% yield) as a white solid. (ESI) m/z: [M+H]+=610.26.

To an oven dried vial was added 66 (13.0 mg, 0.021 mmol, 1 eq) and MC-GGF-OH (12.6 mg, 0.027 mmol, 1.25 eq) under Ar. Anhydrous DMF (0.38 mL) was added to the vial to yield a solution. DMTMM (11.8 mg, 0.043 mmol, 2 eq) was added to the vial and briefly sonicated to yield a suspension. The vial was stirred at room temperature for 0.5 h whereupon the LCMS indicated 66 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL16 (10.4 mg, 46% yield) as a white solid. (ESI) m/z: [M+H]+=1064.44.

### Example 24: Synthesis of PL17

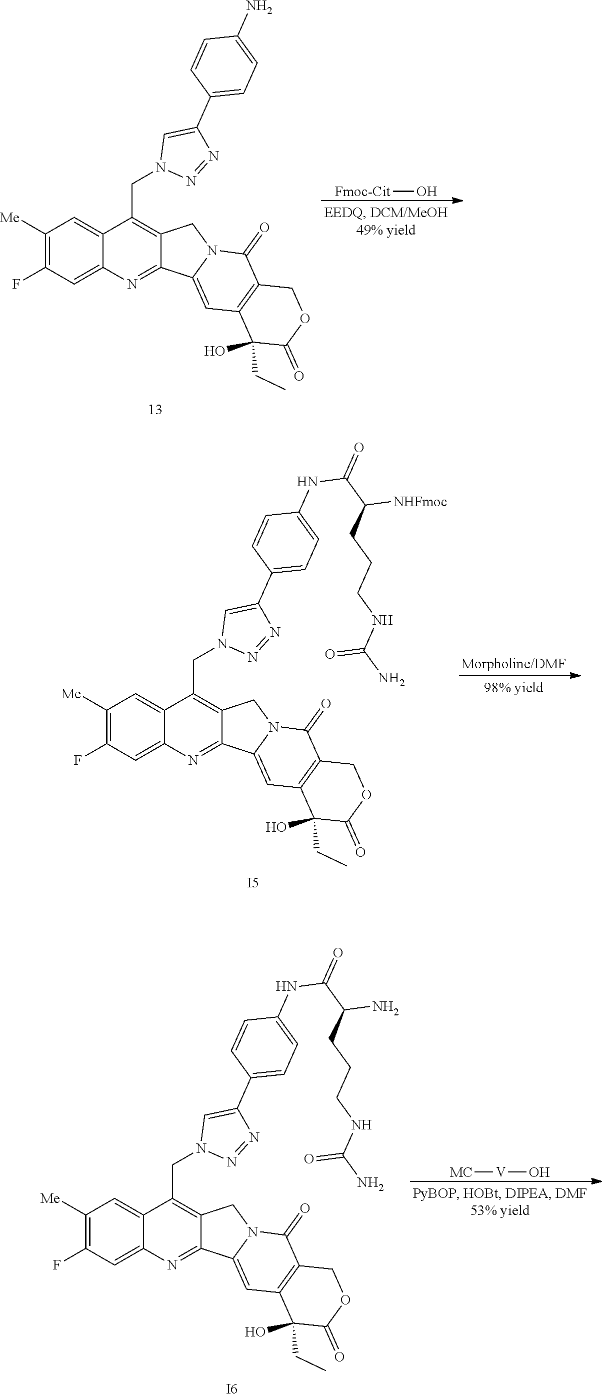

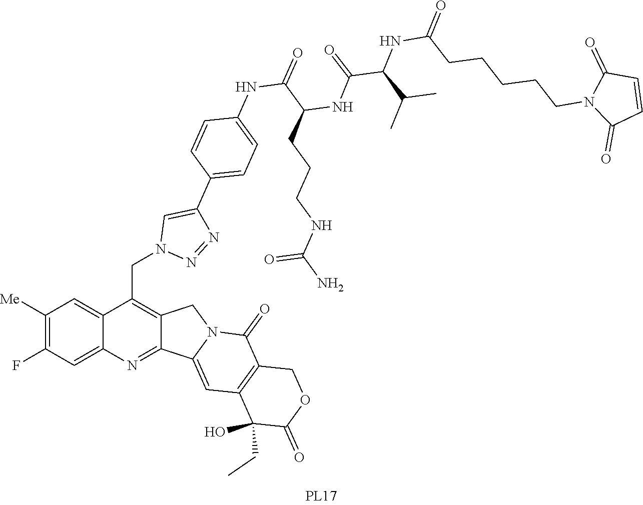 [SMILES: CC[C@@]1(O)C(=O)OCc2c1cc1n(c2=O)Cc2c-1nc1cc(F)c(C)cc1c2Cn1cc(-c2ccc(NC(=O)[C@H](CCCNC(N)=O)NC(=O)[C@@H](NC(=O)CCCCCN3C(=O)C=CC3=O)C(C)C)cc2)nn1]

To an oven dried vial was added Fmoc-Cit-OH (49.8 mg, 0.125 mmol, 2 eq) with (20% MeOH/DCM) (8.35 mL). To the vial was added I3 (35 mg, 0.063 mmol, 1 eq) followed by EEDQ (31 mg, 0.125 mmol, 2 eq) and stirred 16 h whereupon LCMS indicated 13 was consumed. The crude reaction was stripped dry via rotary evaporation, redissolved in DMF (1 mL), and purified via reverse phase (Teledyne Gemini (5 μm C18 110 Å 150×30 mm) with (5% ACN+0.05% TFA)/(H₂O+0.05% TFA) to 95% (ACN+0.05% TFA)/(H₂O+0.05% TFA) gradient). The product was recovered by lyophilization to obtain 15 (28.4 mg, 49% yield) as a white solid. (ESI) m/z: [M+H]+=932.50.

To an oven dried vial was added I5 (27 mg, 0.029 mmol, 1 eq) and dissolved in (1:4) (Morpholine:DMF) (0.415 mL) and stirred at room temperature under Ar. LCMS at 60 minutes indicated that 15 was consumed. The crude reaction was precipitated with diethyl ether (4.5 mL) and the solid was obtained by centrifugation. The solid was washed with diethyl ether (1×) (4.5 mL) and the solid was obtained by centrifugation. This obtained solid was redissolved in (1:1) (H₂O:ACN) and lyophilized to give 16 (20.1 mg, 98% yield) as a white solid. (ESI) m/z: [M+H]+=710.49.

To an oven dried vial was added I6 (12.7 mg, 0.018 mmol, 1 eq) and MC-V-OH (11.1 mg, 0.036 mmol, 2 eq) under Ar. Anhydrous DMF (0.33 mL) was added to the vial to yield a solution, followed by HOBt (4.8 mg, 0.036 mmol, 2 eq). DIPEA (6 uL, 0.036 mmol, 2 eq) was added to the vial, followed by PyBOP (14 mg, 0.027 mmol, 1.5 eq). The vial was stirred at room temperature for 0.5 h whereupon the LCMS indicated I6 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL17 (9.4 mg, 53% yield) as a white solid. (ESI) m/z: [M+H]+=1002.16.

### Example 25: Synthesis of PL18

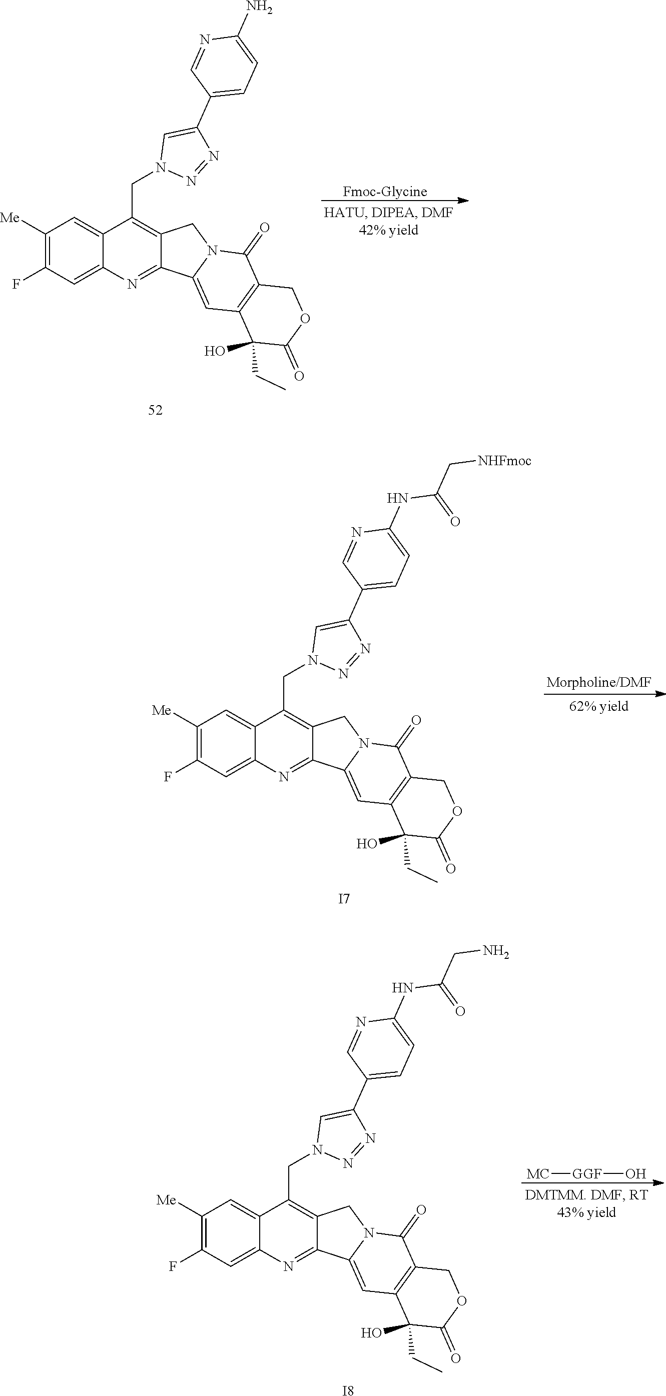

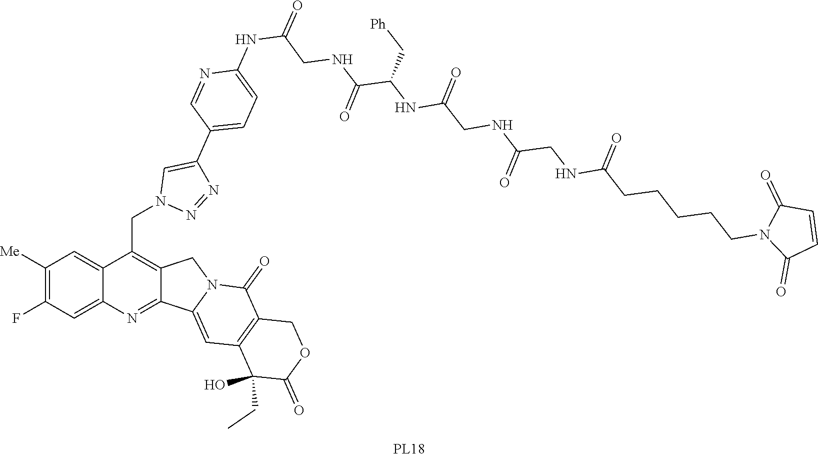 [SMILES: CC[C@@]1(O)C(=O)OCc2c1cc1n(c2=O)Cc2c-1nc1cc(F)c(C)cc1c2Cn1cc(-c2ccc(NC(=O)CNC(=O)[C@H](Cc3ccccc3)NC(=O)CNC(=O)CNC(=O)CCCCCN3C(=O)C=CC3=O)nc2)nn1]

To an oven dried vial was added Fmoc-glycine (36.6 mg, 0.123 mmol, 2 eq) with Anhydrous DMF (1.76 mL). To the vial was added DIPEA (57.1 uL, 0.328 mmol, 4 eq) followed by HATU (46.8 mg, 0.123 mmol, 2 eq) and stirred 10 minutes at room temperature. After ten minutes, 52 (23 mg, 0.041 mmol, 1 eq) was added to the vial and stirred at room temperature for 16 h whereupon LCMS indicated 52 was 80% consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain 17 (14.3 mg, 42% yield) as a white solid. (ESI) m/z: [M+H]+=833.39.

To an oven dried vial was added I7 (14 mg, 0.017 mmol, 1 eq) and dissolved in (1:4) (Morpholine:DMF) (0.245 mL) and stirred at room temperature under Ar. LCMS at 60 minutes indicated that 17 was consumed. The crude reaction was precipitated with diethyl ether (2.5 mL) and the solid was obtained by centrifugation. The solid was washed with diethyl ether (1×) (2.5 mL) and the solid was obtained by centrifugation. This obtained solid was redissolved in (1:1) (H₂O:ACN) and lyophilized to give 18 (6.5 mg, 62% yield) as a white solid. (ESI) m/z: [M+H]+=611.30.

To an oven dried vial was added I8 (7.0 mg, 0.011 mmol, 1 eq) and MC-GGF-OH (6.3 mg, 0.013 mmol, 1.25 eq) under Ar. Anhydrous DMF (1.9 mL) was added to the vial to yield a solution. DMTMM (5.9 mg, 0.021 mmol, 2 eq) was added to the vial and briefly sonicated to yield a suspension. The vial was stirred at room temperature for 0.5 h whereupon the LCMS indicated I8 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL18 (4.9 mg, 43% yield) as a white solid. (ESI) m/z: [M+H]+=1065.54.

### Example 26: Synthesis of PL19

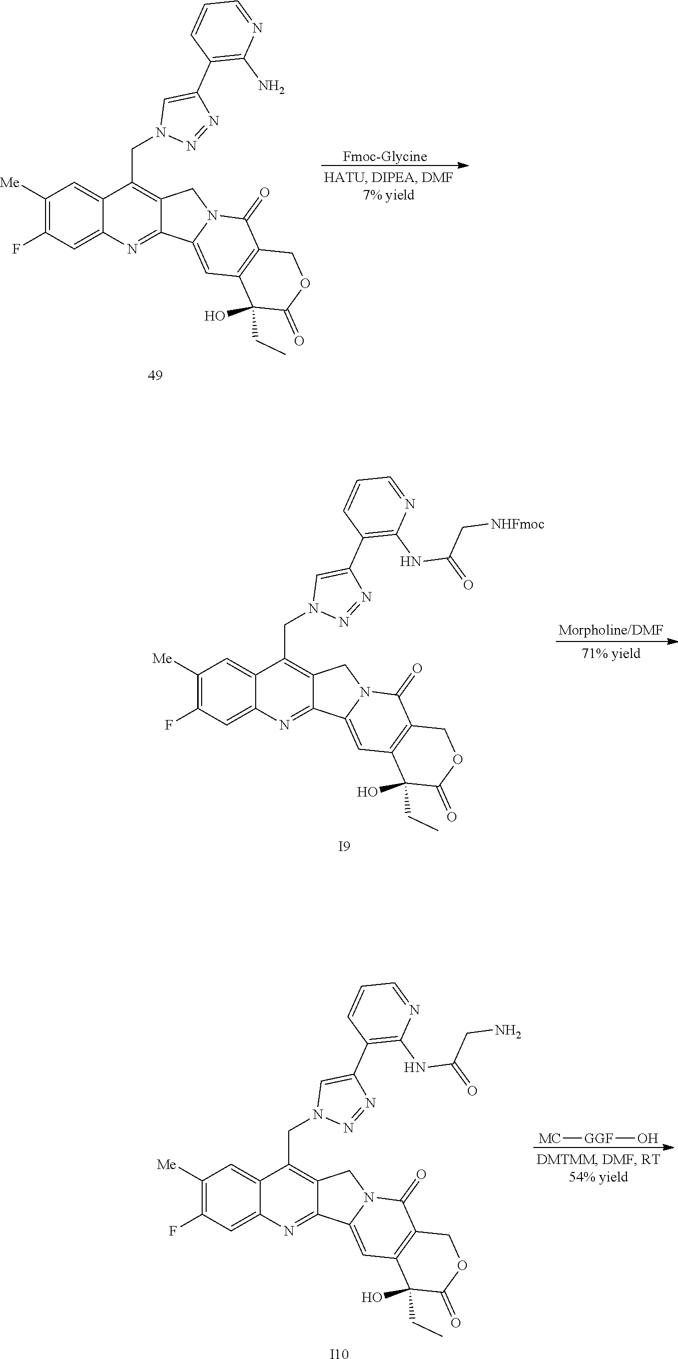

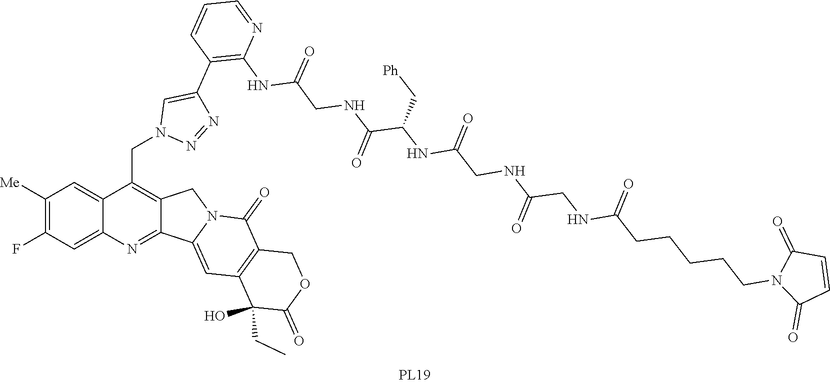 [SMILES: CC[C@@]1(O)C(=O)OCc2c1cc1n(c2=O)Cc2c-1nc1cc(F)c(C)cc1c2Cn1cc(-c2cccnc2NC(=O)CNC(=O)[C@H](Cc2ccccc2)NC(=O)CNC(=O)CNC(=O)CCCCCN2C(=O)C=CC2=O)nn1]

To an oven dried vial was added Fmoc-glycine (27 mg, 0.049 mmol, 2 eq) with Anhydrous DMF (1.41 mL). To the vial was added DIPEA (52 uL, 0.296 mmol, 4 eq) followed by HATU (37.5 mg, 0.099 mmol, 2 eq) and stirred 10 minutes at room temperature. After ten minutes, 49 (27 mg, 0.049 mmol, 1 eq) was added to the vial and stirred at room temperature for 48 h whereupon LCMS indicated 49 was 10% consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain 19 (2.8 mg, 7% yield) as a white solid. (ESI) m/z: [M+H]+=833.34.

To an oven dried vial was added I9 (3 mg, 0.003 mmol, 1 eq) and dissolved in (1:4) (Morpholine:DMF) (0.048 mL) and stirred at room temperature under Ar. LCMS at 60 minutes indicated that 19 was consumed. The crude reaction was precipitated with diethyl ether (1 mL) and the solid was obtained by centrifugation. The solid was washed with diethyl ether (1×) (1 mL) and the solid was obtained by centrifugation. This obtained solid was redissolved in (1:1) (H₂O:ACN) and lyophilized to give 110 (1.5 mg, 71% yield) as a white solid. (ESI) m/z: [M+H]+=611.19.

To an oven dried vial was added I10 (2 mg, 0.002 mmol, 1 eq) and MC-GGF-OH (1.5 mg, 0.003 mmol, 1.25 eq) under Ar. Anhydrous DMF (0.44 mL) was added to the vial to yield a solution. DMTMM (1.4 mg, 0.005 mmol, 2 eq) was added to the vial and briefly sonicated to yield a suspension. The vial was stirred at room temperature for 0.5 h whereupon the LCMS indicated I10 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL19 (1.4 mg, 54% yield) as a white solid. (ESI) m/z: [M+H]+=1065.47.

### Example 27: Synthesis of PL20

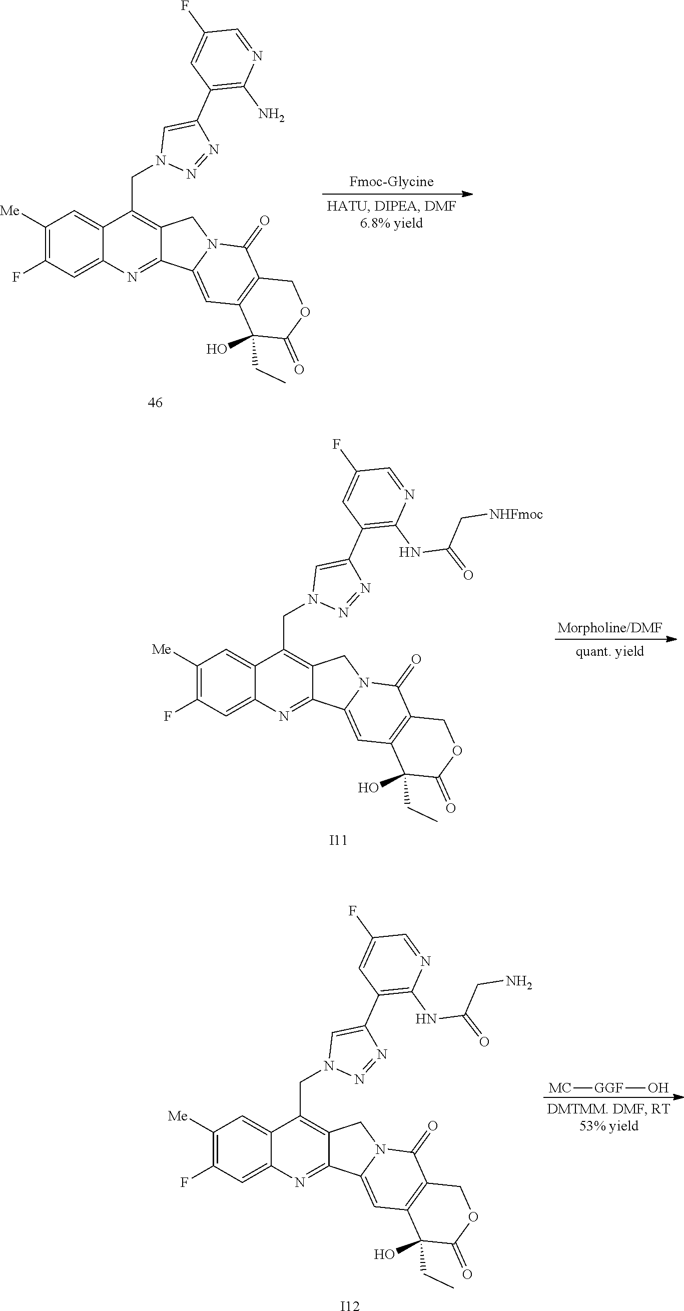

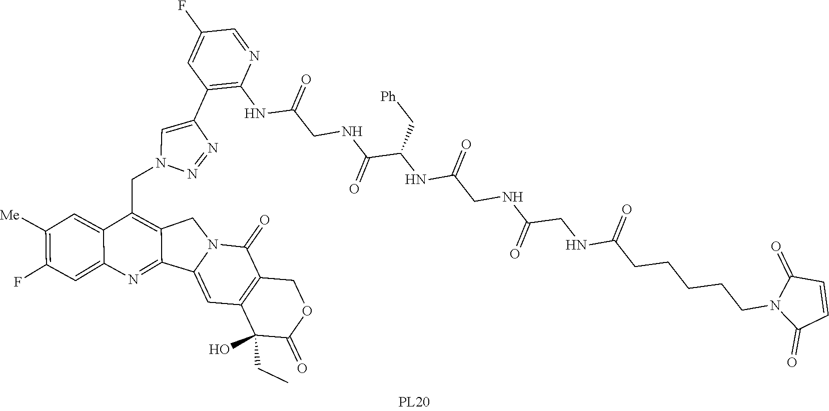 [SMILES: CC[C@@]1(O)C(=O)OCc2c1cc1n(c2=O)Cc2c-1nc1cc(F)c(C)cc1c2Cn1cc(-c2cc(F)cnc2NC(=O)CNC(=O)[C@H](Cc2ccccc2)NC(=O)CNC(=O)CNC(=O)CCCCCN2C(=O)C=CC2=O)nn1]

To an oven dried vial was added Fmoc-glycine (11.4 mg, 0.038 mmol, 2 eq) with Anhydrous DMF (0.55 mL). To the vial was added DIPEA (13 uL, 0.077 mmol, 4 eq) followed by HATU (14.6 mg, 0.038 mmol, 2 eq) and stirred 10 minutes at room temperature. After ten minutes, 46 (11 mg, 0.019 mmol, 1 eq) was added to the vial and stirred at room temperature for 48 h whereupon LCMS indicated 46 was 20% consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain I11 (1 mg, 7% yield) as a white solid. (ESI) m/z: [M+H]+=851.62.

To an oven dried vial was added I11 (1 mg, 0.016 mmol, 1 eq) and dissolved in (1:4) (Morpholine:DMF) (0.017 mL) and stirred at room temperature under Ar. LCMS at 60 minutes indicated that 111 was consumed. The crude reaction was precipitated with diethyl ether (1 mL) and the solid was obtained by centrifugation. The solid was washed with diethyl ether (1×) (1 mL) and the solid was obtained by centrifugation. This obtained solid was redissolved in (1:1) (H₂O:ACN) and lyophilized to give 112 (1 mg, quant % yield) as a white solid. (ESI) m/z: [M+H]+=629.22.

To an oven dried vial was added I12 (1 mg, 0.002 mmol, 1 eq) and MC-GGF-OH (1 mg, 0.002 mmol, 1.25 eq) under Ar. Anhydrous DMF (0.05 mL) was added to the vial to yield a solution. DMTMM (1 mg, 0.003 mmol, 2 eq) was added to the vial and briefly sonicated to yield a suspension. The vial was stirred at room temperature for 2 h whereupon the LCMS indicated I12 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL20 (0.9 mg, 53% yield) as a white solid. (ESI) m/z: [M+H]+=1083.40.

### Example 28: Synthesis of PL21

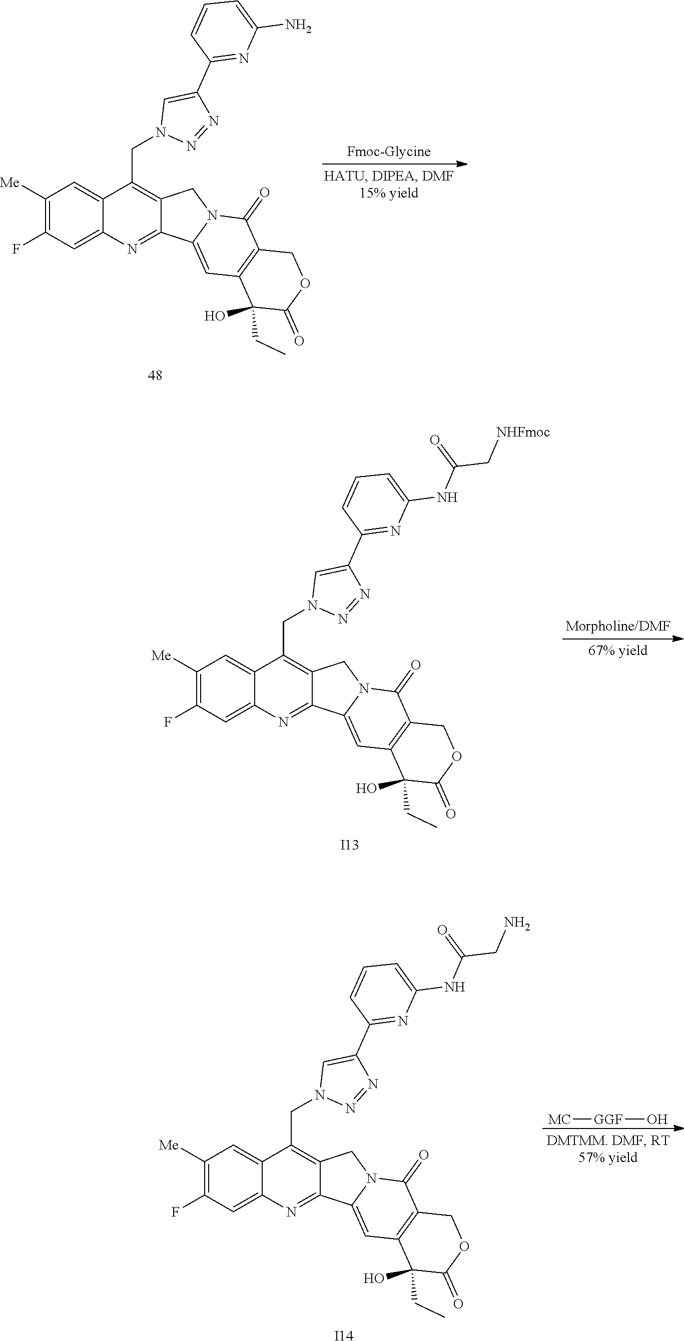

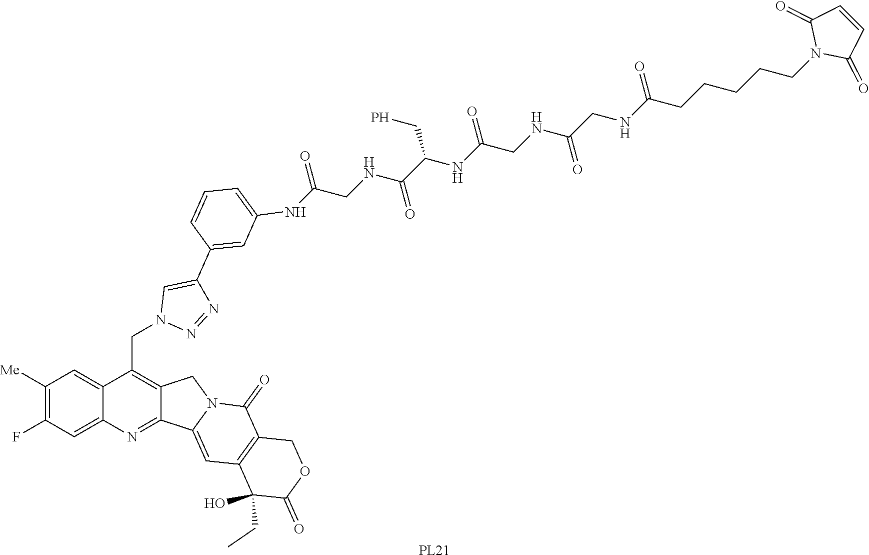

To an oven dried vial was added Fmoc-glycine (14.7 mg, 0.049 mmol, 3.5 eq) with Anhydrous DMF (0.70 mL). To the vial was added DIPEA (17 uL, 0.099 mmol, 7 eq) followed by HATU (18.8 mg, 0.049 mmol, 3.5 eq) and stirred 10 minutes at room temperature. After ten minutes, 48 (8 mg, 0.014 mmol, 1 eq) was added to the vial and stirred at room temperature for 2 h whereupon LCMS indicated 46 was 5% consumed. The reaction was stirred for 16 h at 40 C, where 46 was 40% consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain 113 (1.7 mg, 15% yield) as a white solid. (ESI) m/z: [M+H]+=833.30.

To an oven dried vial was added I13 (2 mg, 0.02 mmol, 1 eq) and dissolved in (1:4) (Morpholine:DMF) (0.027 mL) and stirred at room temperature under Ar. LCMS at 60 minutes indicated that 113 was consumed. The crude reaction was precipitated with diethyl ether (1 mL) and the solid was obtained by centrifugation. The solid was washed with diethyl ether (1×) (1 mL) and the solid was obtained by centrifugation. This obtained solid was redissolved in (1:1) (H₂O:ACN) and lyophilized to give 114 (0.8 mg, 67% yield) as a white solid. (ESI) m/z: [M+H]+=611.20.

To an oven dried vial was added I14 (1 mg, 0.001 mmol, 1 eq) and MC-GGF-OH (2 mg, 0.002 mmol, 1.25 eq) under Ar. Anhydrous DMF (0.04 mL) was added to the vial to yield a solution. DMTMM (1 mg, 0.003 mmol, 2 eq) was added to the vial and briefly sonicated to yield a suspension. The vial was stirred at room temperature for 0.5 h whereupon the LCMS indicated I14 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL21 (0.8 mg, 57% yield) as a white solid. (ESI) m/z: [M+H]+=1065.41.

### Example 29: Synthesis of PL22

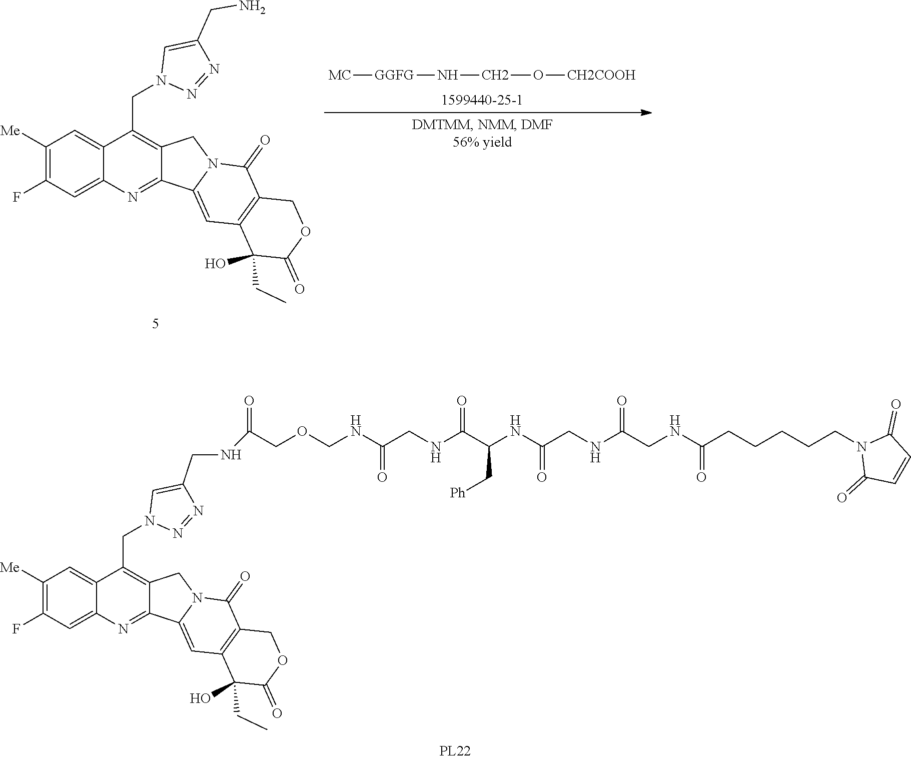

To an oven dried vial was added 5 (10.0 mg, 0.020 mmol, 1 eq) and MC-GGFG-NH—CH₂—O—CH₂COOH (13.5 mg, 0.022 mmol, 1.11 eq) under Ar. Anhydrous DMF (0.61 mL) was added to the vial to yield a solution. NMM (3 uL, 0.025 mmol, 1.25 eq) was added to the vial followed by DMTMM (10.9 mg, 0.040 mmol, 2 eq) and briefly sonicated to yield a suspension. The vial was stirred at room temperature for 0.5 h whereupon the LCMS indicated 33 was consumed. The crude reaction was purified via reverse (Teledyne Gemini (5 μm C18 110 Å 150×30 mm) with (5% ACN)/(H₂O) to 95% (ACN)/(H₂O) gradient). The product was recovered by lyophilization to obtain PL14 (12.0 mg, 56% yield) as a white solid. (ESI) m/z: [M+H]+=1089.61.

### Example 30: Synthesis of PL23

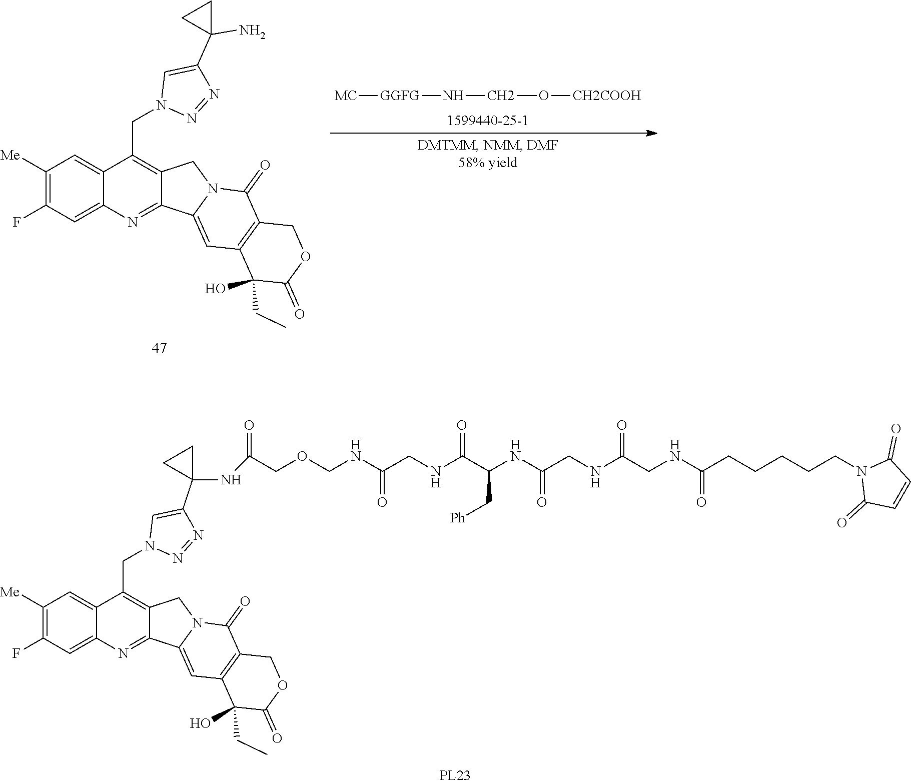

To an oven dried vial was added 47 (11.0 mg, 0.021 mmol, 1 eq) and MC-GGFG-NH—CH₂—O—CH₂COOH (14.4 mg, 0.023 mmol, 1.11 eq) under Ar. Anhydrous DMF (0.65 mL) was added to the vial to yield a solution. NMM (3 uL, 0.026 mmol, 1.25 eq) was added to the vial followed by DMTMM (11.7 mg, 0.042 mmol, 2 eq) and briefly sonicated to yield a suspension. The vial was stirred at room temperature for 0.5 h whereupon the LCMS indicated 47 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL23 (13.6 mg, 58% yield) as a white solid. (ESI) m/z: [M+H]+=1115.93.

### Example 31: Synthesis of PL24

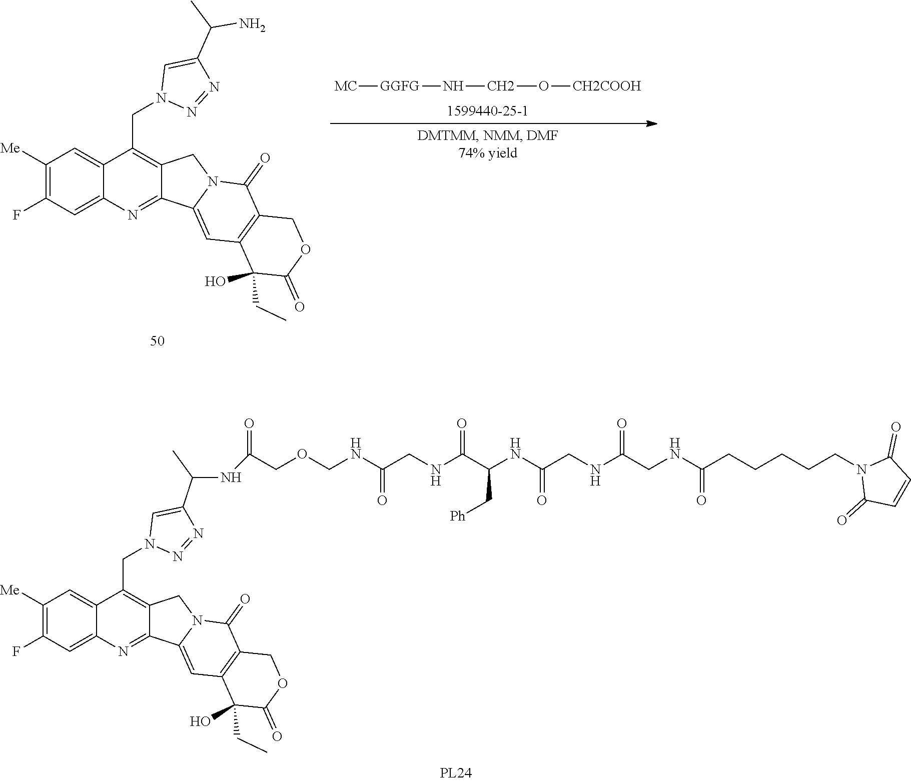

To an oven dried vial was added 50 (11.0 mg, 0.021 mmol, 1 eq) and MC-GGFG-NH—CH₂—O—CH₂COOH (14.4 mg, 0.023 mmol, 1.11 eq) under Ar. Anhydrous DMF (0.65 mL) was added to the vial to yield a solution. NMM (3 uL, 0.026 mmol, 1.25 eq) was added to the vial followed by DMTMM (11.6 mg, 0.042 mmol, 2 eq) and briefly sonicated to yield a suspension. The vial was stirred at room temperature for 0.5 h whereupon the LCMS indicated 50 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL24 (17.2 mg, 74% yield) as a white solid. (ESI) m/z: [M+H]+=1103.15.

### Example 32: Synthesis of PL25

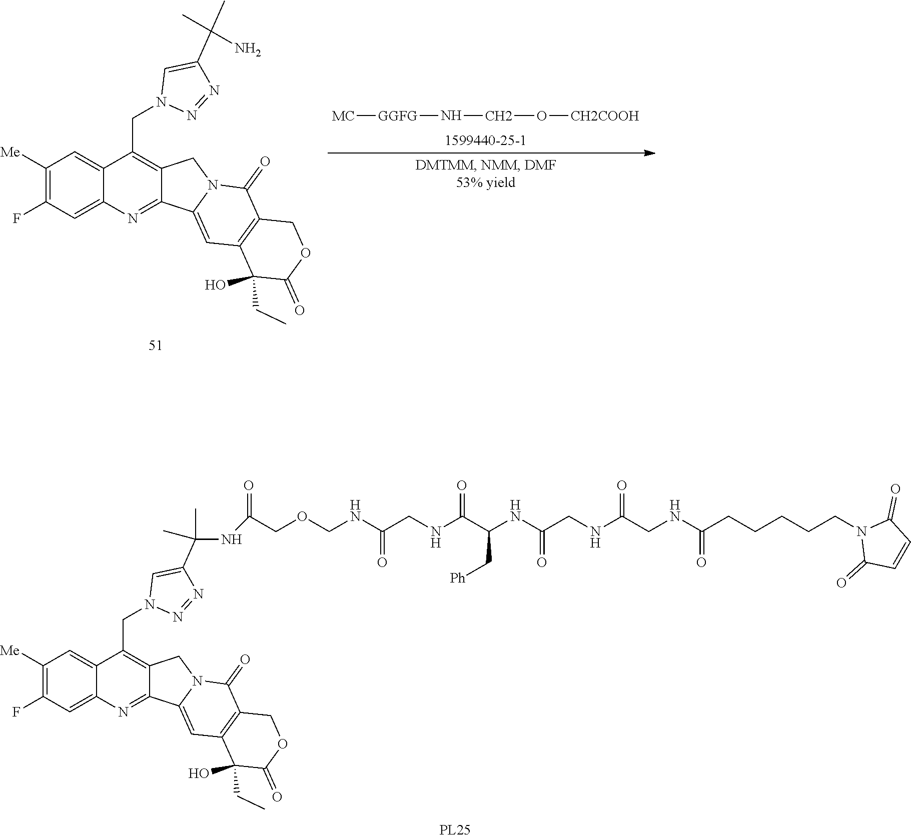

To an oven dried vial was added 51 (9.0 mg, 0.017 mmol, 1 eq) and MC-GGFG-NH—CH₂—O—CH₂COOH (11.7 mg, 0.019 mmol, 1.11 eq) under Ar. Anhydrous DMF (0.53 mL) was added to the vial to yield a solution. NMM (2 uL, 0.021 mmol, 1.25 eq) was added to the vial followed by DMTMM (9.5 mg, 0.034 mmol, 2 eq) and briefly sonicated to yield a suspension. The vial was stirred at room temperature for 0.5 h whereupon the LCMS indicated 51 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL25 (10.2 mg, 53% yield) as a white solid. (ESI) m/z: [M+H]+=1117.47.

### Example 33: Synthesis of PL26

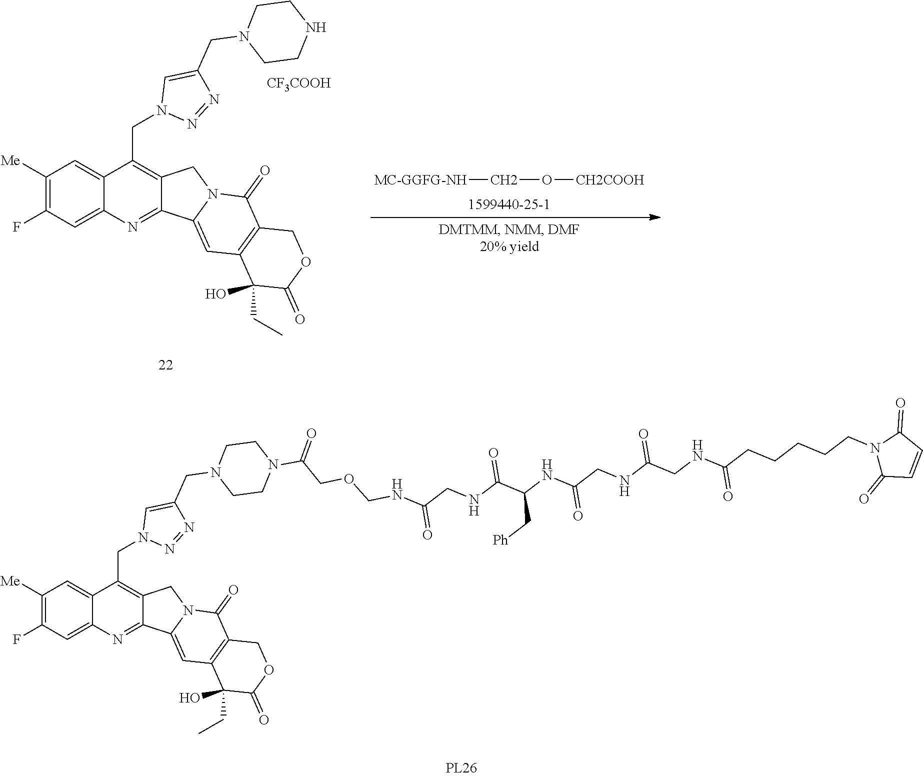

To an oven dried vial was added 22 (8.0 mg, 0.015 mmol, 1 eq) and MC-GGFG-NH—CH₂—O—CH₂COOH (10.3 mg, 0.017 mmol, 1.11 eq) under Ar. Anhydrous DMF (0.46 mL) was added to the vial to yield a solution. NMM (2 uL, 0.019 mmol, 1.25 eq) was added to the vial followed by DMTMM (8.3 mg, 0.030 mmol, 2 eq) and briefly sonicated to yield a suspension. The vial was stirred at room temperature for 0.5 h whereupon the LCMS indicated 22 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL26 (3.4 mg, 20% yield) as a white solid. (ESI) m/z: [M+H]+=1158.85.

### Example 34: Synthesis of PL27

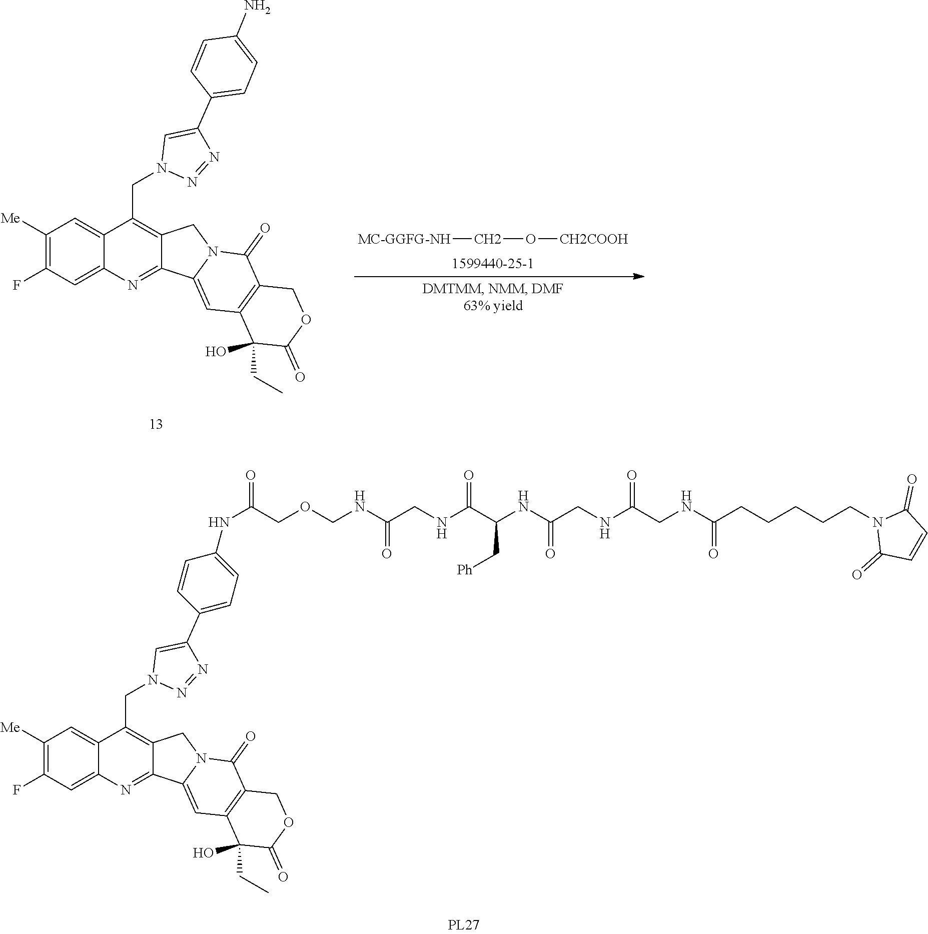

To an oven dried vial was added I3 (10.0 mg, 0.017 mmol, 1 eq) and MC-GGFG-NH—CH₂—O—CH₂COOH (11.9 mg, 0.019 mmol, 1.11 eq) under Ar. Anhydrous DMF (0.54 mL) was added to the vial to yield a solution. NMM (2 uL, 0.022 mmol, 1.25 eq) was added to the vial followed by DMTMM (9.6 mg, 0.034 mmol, 2 eq) and briefly sonicated to yield a suspension. The vial was stirred at room temperature for 0.5 h whereupon the LCMS indicated I3 was consumed. The crude reaction was purified via reverse (Teledyne Gemini (5 μm C18 110 Å 150×30 mm) with (5% ACN)/(H₂O) to 95% (ACN)/(H₂O) gradient). The product was recovered by lyophilization to obtain PL27 (12.6 mg, 63% yield) as a white solid. (ESI) m/z: [M+H]+=1151.45.

### Example 35: Synthesis of PL28

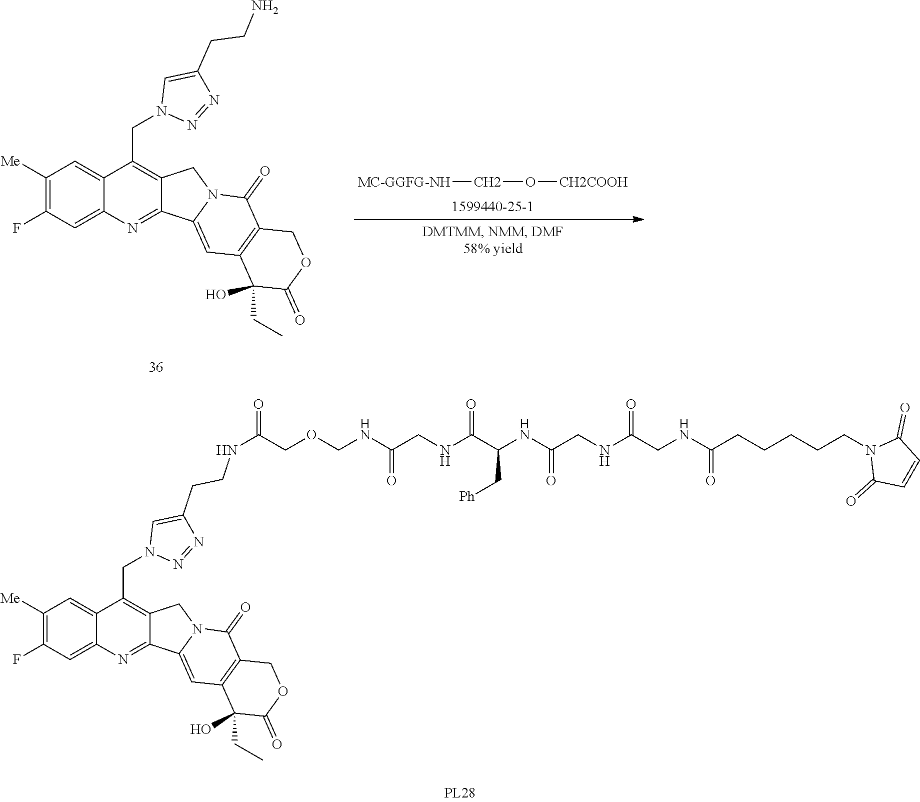

To an oven dried vial was added 36 (9.0 mg, 0.018 mmol, 1 eq) and MC-GGFG-NH—CH₂—O—CH₂COOH (12.5 mg, 0.020 mmol, 1.11 eq) under Ar. Anhydrous DMF (0.56 mL) was added to the vial to yield a solution. NMM (3 uL, 0.023 mmol, 1.25 eq) was added to the vial followed by DMTMM (10.1 mg, 0.036 mmol, 2 eq) and briefly sonicated to yield a suspension. The vial was stirred at room temperature for 0.5 h whereupon the LCMS indicated 36 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL28 (11.7 mg, 74% yield) as a white solid. (ESI) m/z: [M+H]+=1103.98.

### Example 36: Synthesis of PL29

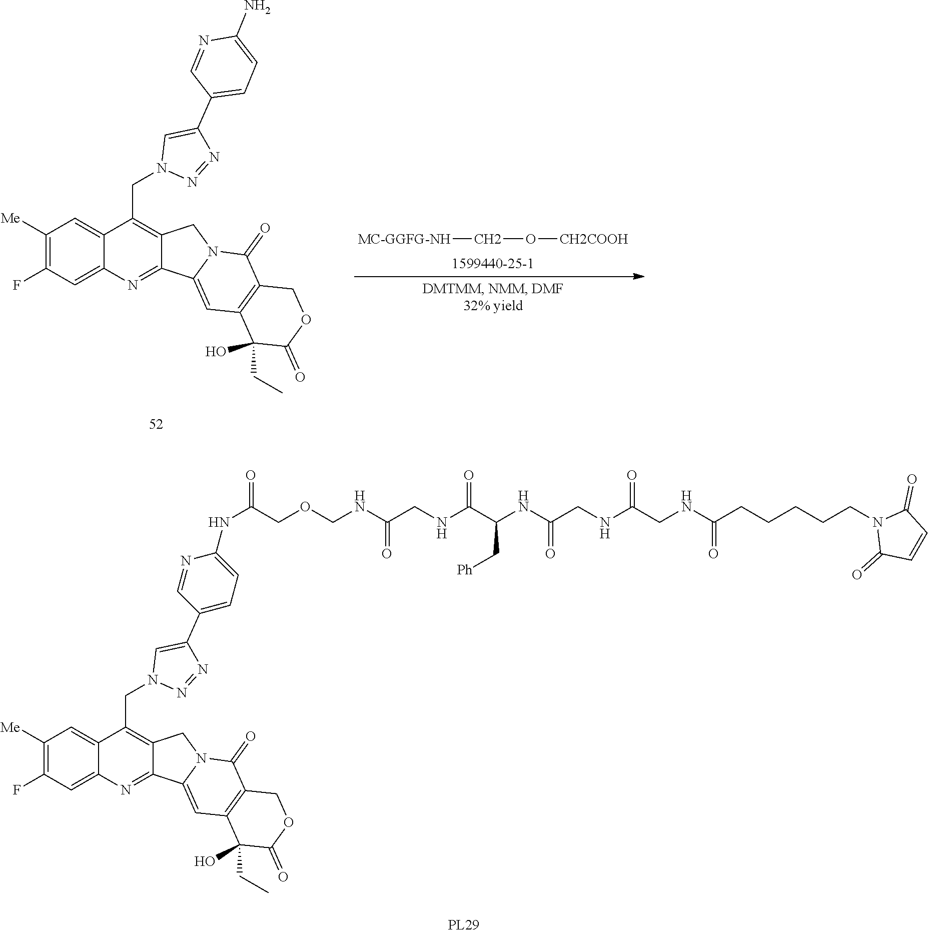

To an oven dried vial was added 52 (10.0 mg, 0.018 mmol, 1 eq) and MC-GGFG-NH—CH₂—O—CH₂COOH (12.6 mg, 0.020 mmol, 1.11 eq) under Ar. Anhydrous DMF (0.57 mL) was added to the vial to yield a solution. NMM (3 uL, 0.023 mmol, 1.25 eq) was added to the vial followed by DMTMM (10.2 mg, 0.037 mmol, 2 eq) and briefly sonicated to yield a suspension. The vial was stirred at room temperature for 16 h whereupon the LCMS indicated 50 was 45% consumed, but the reaction stalled. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL29 (6.7 mg, 32% yield) as a white solid. (ESI) m/z: [M+H]+=1152.93.

### Example 37: Synthesis of PL30

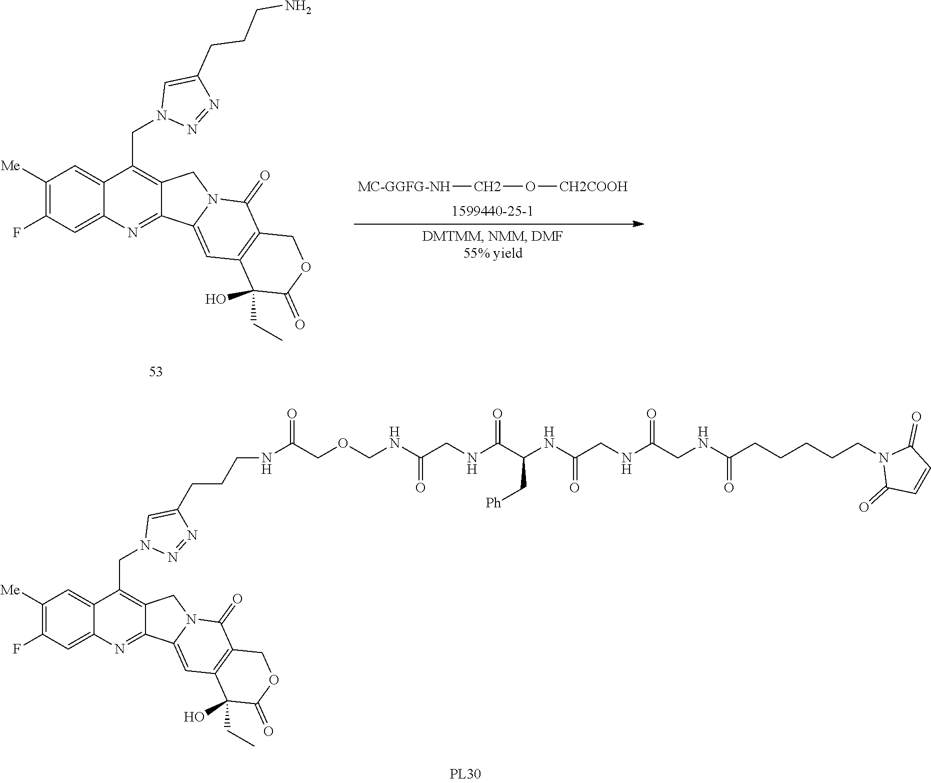

To an oven dried vial was added 53 (21 mg, 0.041 mmol, 1 eq) and MC-GGFG-NH—CH₂—O—CH₂COOH (28.2 mg, 0.046 mmol, 1.11 eq) under Ar. Anhydrous DMF (1.27 mL) was added to the vial to yield a solution. NMM (6 uL, 0.052 mmol, 1.25 eq) was added to the vial followed by DMTMM (22.8 mg, 0.083 mmol, 2 eq) and briefly sonicated to yield a suspension. The vial was stirred at room temperature for 0.5 h whereupon the LCMS indicated 53 was consumed. The crude reaction was purified via reverse (Teledyne Gemini (5 μm C18 110 Å 150×30 mm) with (5% ACN)/(H₂O) to 95% (ACN)/(H₂O) gradient). The product was recovered by lyophilization to obtain PL30 (25.2 mg, 55% yield) as a white solid. (ESI) m/z: [M+H]+=1117.75.

### Example 38: Synthesis of PL31

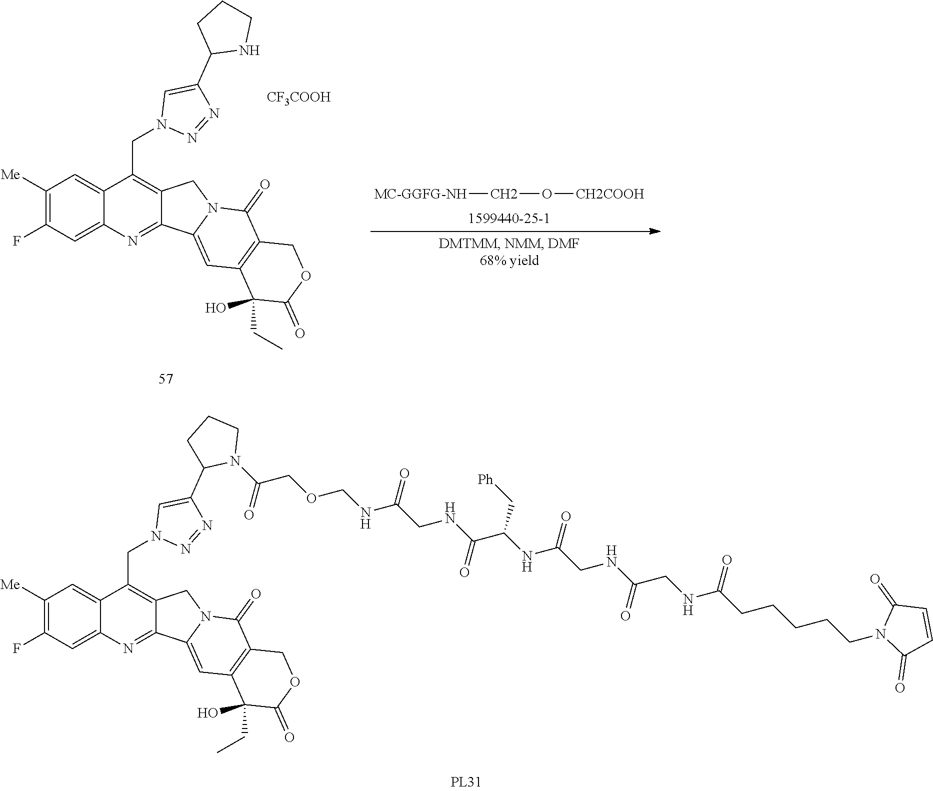

To an oven dried vial was added 57 (11.1 mg, 0.017 mmol, 1 eq) and MC-GGFG-NH—CH₂—O—CH₂COOH (11.5 mg, 0.019 mmol, 1.11 eq) under Ar. Anhydrous DMF (0.52 mL) was added to the vial to yield a solution. NMM (2 uL, 0.021 mmol, 1.25 eq) was added to the vial followed by DMTMM (9.3 mg, 0.034 mmol, 2 eq) and briefly sonicated to yield a suspension. The vial was stirred at room temperature for 0.5 h whereupon the LCMS indicated 57 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL31 (12.8 mg, 68% yield) as a white solid. (ESI) m/z: [M+H]+=1129.72.

### Example 39: Synthesis of PL32

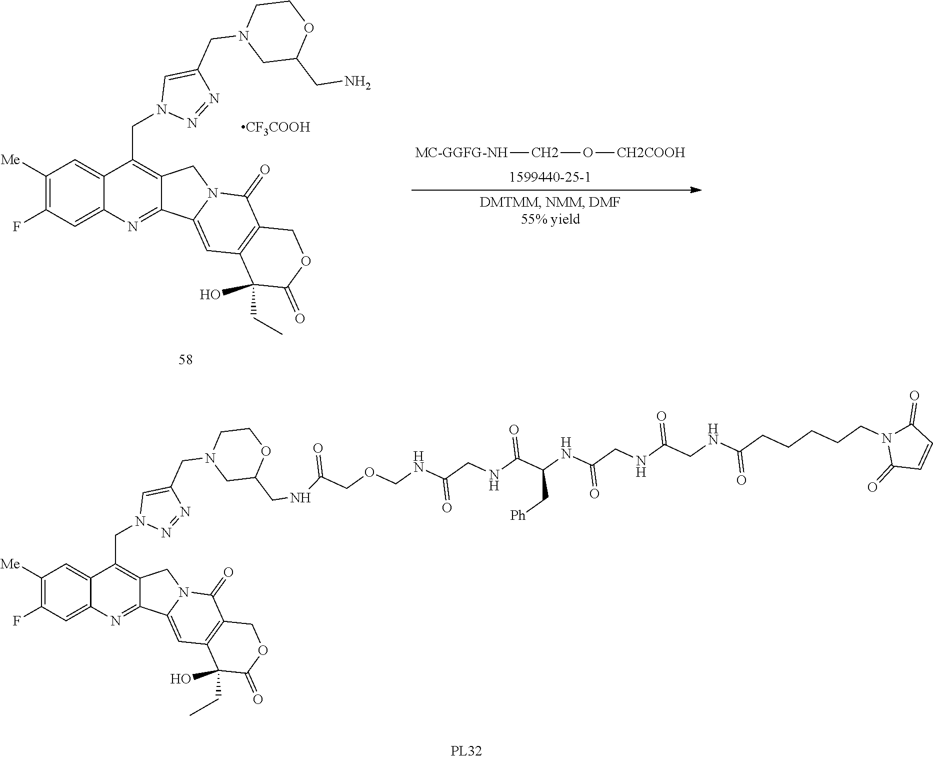

To an oven dried vial was added 58 (13.0 mg, 0.018 mmol, 1 eq) and MC-GGFG-NH—CH₂—O—CH₂COOH (12.6 mg, 0.021 mmol, 1.11 eq) under Ar. Anhydrous DMF (0.57 mL) was added to the vial to yield a solution. NMM (3 uL, 0.023 mmol, 1.25 eq) was added to the vial followed by DMTMM (10.2 mg, 0.037 mmol, 2 eq) and briefly sonicated to yield a suspension. The vial was stirred at room temperature for 0.5 h whereupon the LCMS indicated 50 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL32 (12.2 mg, 55% yield) as a white solid. (ESI) m/z: [M+H]+=1188.67.

### Example 40: Synthesis of PL33

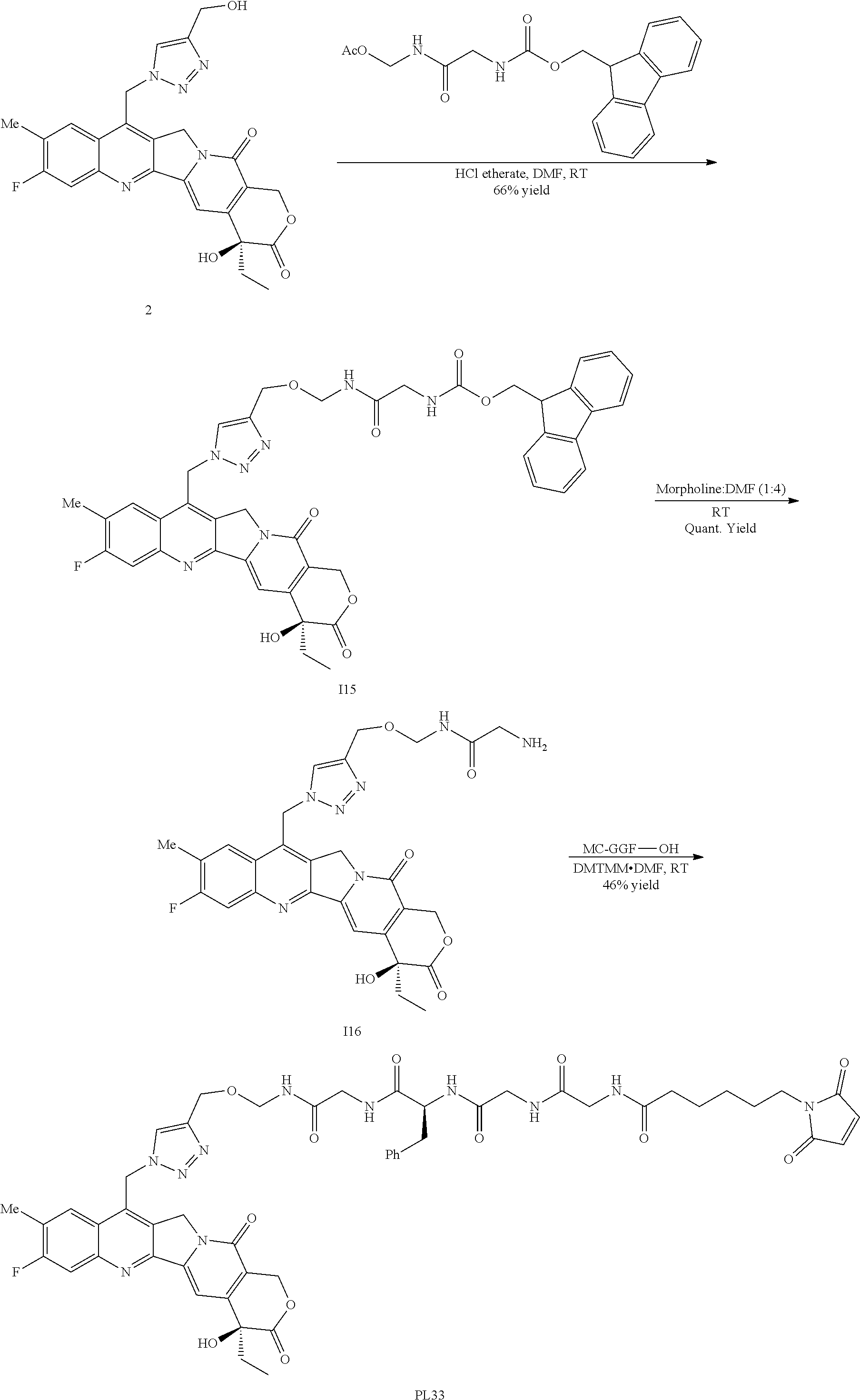

To an oven dried vial was added 2 (249 mg, 0.506 mmol, 1 eq) and OAc—CH₂—NH-Gly-Fmoc (186 mg, 0.5064 mmol, 1 eq) and suspended in Anhydrous DMF (7.13 mL) with a brief sonication under Ar. To the vial, 1M HCl etherate (0.304 mL, 0.608 mmol, 1.2 eq) was added dropwise. The vial was stirred for 1.5 h at room temperature and then OAc—CH₂—NH-Gly-Fmoc (186 mg, 0.5064 mmol, 1 eq) was added (total eq=2 thus far). The vial was stirred for 1.5 h at room temperature and then OAc—CH₂—NH-Gly-Fmoc (186 mg, 0.5064 mmol, 1 eq) was added (total eq=3 thus far). The vial was stirred for 1.5 h at room temperature and then OAc—CH₂—NH-Gly-Fmoc (186 mg, 0.5064 mmol, 1 eq) was added (total eq=4 thus far). The vial was stirred for 1.5 h at room temperature and then OAc—CH₂—NH-Gly-Fmoc (186 mg, 0.5064 mmol, 1 eq) was added (total eq=5 thus far). The vial was stirred for 16 h at room temperature. LCMS indicated that the reaction had stalled, and the reaction was purified via reverse phase (Teledyne 150 g C18Aq with (5% ACN+0.05% FA)/(H₂O+0.05% FA) to 95% (ACN+0.05% FA)/(H₂O+0.05% FA) gradient). The product was recovered by lyophilization to obtain 115 (270 mg, 66% yield) as a white solid. (ESI) m/z: [M+H]+=800.12.

To an oven dried vial was added I15 (270 mg, 0.336 mmol, 1 eq) and dissolved in (1:4) (Morpholine:DMF) (4.803 mL) and stirred at room temperature under Ar. LCMS at 120 minutes indicated that 115 was consumed. The crude reaction was precipitated with diethyl ether (50 mL) and the solid was obtained by centrifugation. The solid was washed with diethyl ether (1×) (50 mL) and the solid was obtained by centrifugation. This obtained solid was redissolved in (1:1) (H₂O:ACN) and lyophilized to give 116 (174 mg, quant % yield) as a white solid. (ESI) m/z: [M+H]+=578.23.

To an oven dried vial was added I16 (174 mg, 0.301 mmol, 1 eq) and MC-GGF-OH (177 mg, 0.376 mmol, 1.25 eq) under Ar. Anhydrous DMF (5.37 mL) was added to the vial to yield a solution. DMTMM (166 mg, 0.601 mmol, 2 eq) was added to the vial and briefly sonicated to yield a suspension. The vial was stirred at room temperature for 2 h whereupon the LCMS indicated I16 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL33 (100 mg, 32% yield) as a white solid. (ESI) m/z: [M+H]+=1032.46.

### Example 41: Synthesis of PL34

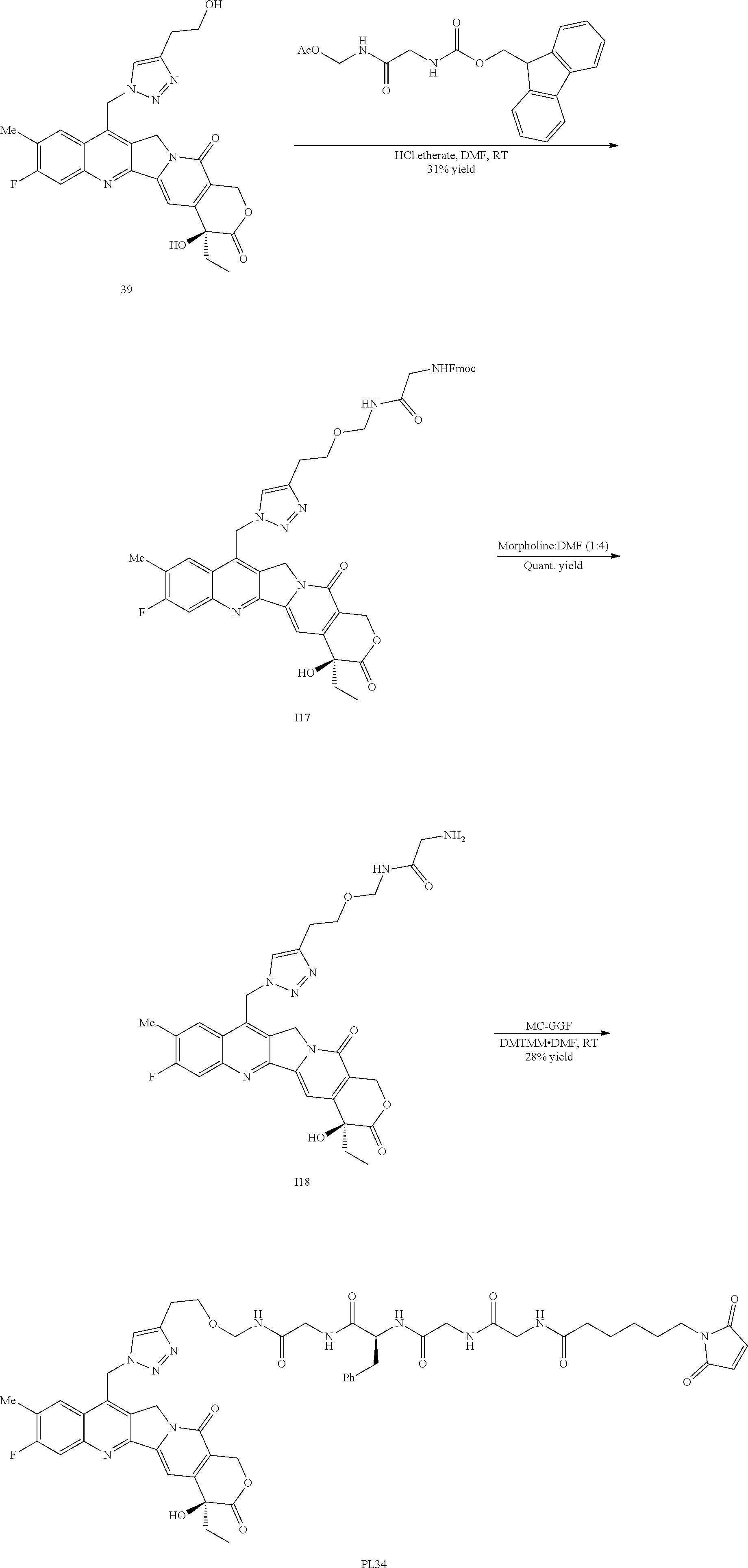

To an oven dried vial was added 39 (28 mg, 0.055 mmol, 1 eq) and OAc—CH₂—NH-Gly-Fmoc (20 mg, 0.055 mmol, 1 eq) and suspended in Anhydrous DMF (0.77 mL) with a brief sonication under Ar. To the vial, 1M HCl etherate (0.076 mL, 0.44 mmol, 8 eq) was added dropwise. The vial was stirred for 1.5 h at room temperature and then OAc—CH₂—NH₂-Gly-Fmoc (20 mg, 0.055 mmol, 1 eq) was added (total eq=2 thus far). The vial was stirred for 1.5 h at room temperature and then OAc—CH₂—NH-Gly-Fmoc (20 mg, 0.055 mmol, 1 eq) was added (total eq=3 thus far). The vial was stirred for 1.5 h at room temperature and then OAc—CH₂—NH-Gly-Fmoc (20 mg, 0.055 mmol, 1 eq) was added (total eq=4 thus far). The vial was stirred for 1.5 h at room temperature and then OAc—CH₂—NH-Gly-Fmoc (20 mg, 0.055 mmol, 1 eq) was added (total eq=5 thus far). The vial was stirred for 16 h at room temperature. LCMS indicated that the reaction had stalled, and the reaction was purified via reverse phase (Teledyne 50 g C18Aq with (5% ACN+0.05% FA)/(H₂O+0.05% FA) to 95% (ACN+0.05% FA)/(H₂O+0.05% FA) gradient). The product was recovered by lyophilization to obtain 117 (13.8 mg, 30.8% yield) as a white solid. (ESI) m/z: [M+H]+=814.31.

To an oven dried vial was added I17 (14 mg, 0.017 mmol, 1 eq) and dissolved in (1:4) (Morpholine:DMF) (0.237 mL) and stirred at room temperature under Ar. LCMS at 60 minutes indicated that 117 was consumed. The crude reaction was precipitated with diethyl ether (2 mL) and the solid was obtained by centrifugation. The solid was washed with diethyl ether (1×) (2 mL) and the solid was obtained by centrifugation. This obtained solid was redissolved in (1:1) (H₂O:ACN) and lyophilized to give 118 (10.4 mg, quant % yield) as a white solid. (ESI) m/z: [M+H]+=592.45.

To an oven dried vial was added I18 (10 mg, 0.018 mmol, 1 eq) and MC-GGF-OH (10 mg, 0.022 mmol, 1.25 eq) under Ar. Anhydrous DMF (3.14 mL) was added to the vial to yield a solution. DMTMM (9.7 mg, 0.035 mmol, 2 eq) was added to the vial and briefly sonicated to yield a suspension. The vial was stirred at room temperature for 0.5 h whereupon the LCMS indicated I18 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL34 (5.1 mg, 28% yield) as a white solid. (ESI) m/z: [M+H]+=1046.44.

### Example 42: Synthesis of PL35

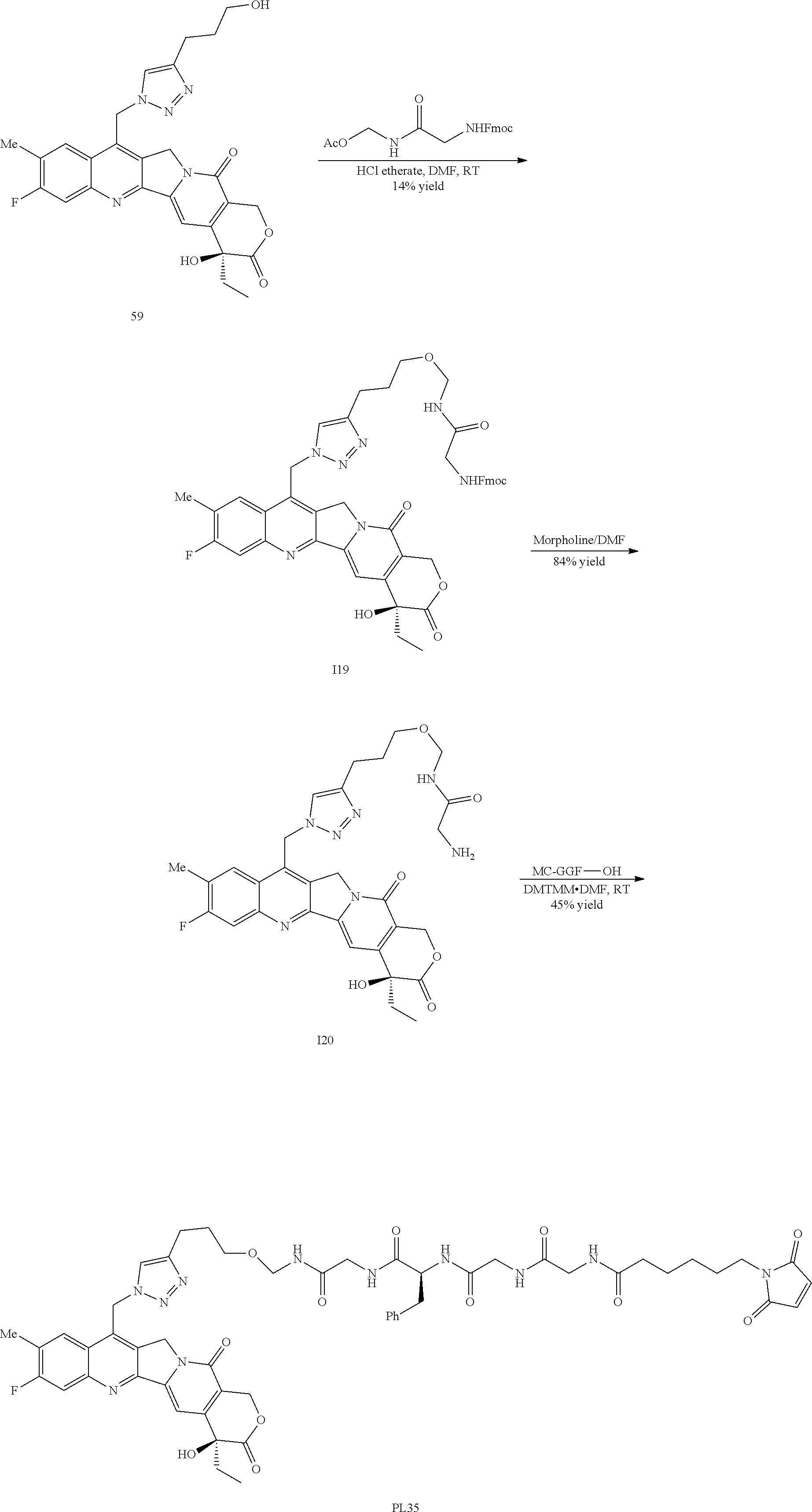

To an oven dried vial was added 59 (26 mg, 0.049 mmol, 1 eq) and OAc—CH₂—NH-Gly-Fmoc (18 mg, 0.049 mmol, 1 eq) and suspended in Anhydrous DMF (0.70 mL) with a brief sonication under Ar. To the vial, 1M HCl etherate (0.083 mL, 0.059 mmol, 1.2 eq) was added dropwise. The vial was stirred for 1.5 h at room temperature and then OAc—CH₂—NH-Gly-Fmoc (18 mg, 0.049 mmol, 1 eq) was added (total eq=2 thus far). The vial was stirred for 1.5 h at room temperature and then OAc—CH₂—NH-Gly-Fmoc (18 mg, 0.049 mmol, 1 eq) was added (total eq=3 thus far). The vial was stirred for 1.5 h at room temperature and then OAc—CH₂—NH-Gly-Fmoc (18 mg, 0.049 mmol, 1 eq) was added (total eq=4 thus far). The vial was stirred for 1.5 h at room temperature and then OAc—CH₂—NH-Gly-Fmoc (18 mg, 0.049 mmol, 1 eq) was added (total eq=5 thus far). The vial was stirred for 16 h at room temperature. LCMS indicated that the reaction had stalled, and the reaction was purified via reverse phase (Teledyne 50 g C18Aq with (5% ACN+0.05% FA)/(H₂O+0.05% FA) to 95% (ACN+0.05% FA)/(H₂O+0.05% FA) gradient). The product was recovered by lyophilization to obtain 119 (6.1 mg, 14% yield) as a white solid. (ESI) m/z: [M+H]+=828.28.

To an oven dried vial was added I19 (6.1 mg, 0.007 mmol, 1 eq) and dissolved in (1:4) (Morpholine:DMF) (0.105 mL) and stirred at room temperature under Ar. LCMS at 60 minutes indicated that 119 was consumed. The crude reaction was precipitated with diethyl ether (1 mL) and the solid was obtained by centrifugation. The solid was washed with diethyl ether (1×) (1 mL) and the solid was obtained by centrifugation. This obtained solid was redissolved in (1:1) (H₂O:ACN) and lyophilized to give 120 (3.8 mg, 84% yield) as a white solid. (ESI) m/z: [M+H]+=606.27.

To an oven dried vial was added I20 (3.8 mg, 0.006 mmol, 1 eq) and MC-GGF-OH (3.7 mg, 0.008 mmol, 1.25 eq) under Ar. Anhydrous DMF (0.2 mL) was added to the vial to yield a solution. DMTMM (3.5 mg, 0.013 mmol, 2 eq) was added to the vial and briefly sonicated to yield a suspension. The vial was stirred at room temperature for 0.5 h whereupon the LCMS indicated I20 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL35 (3.0 mg, 45% yield) as a white solid. (ESI) m/z: [M+H]+=1060.86.

### Example 43: Synthesis of PL36

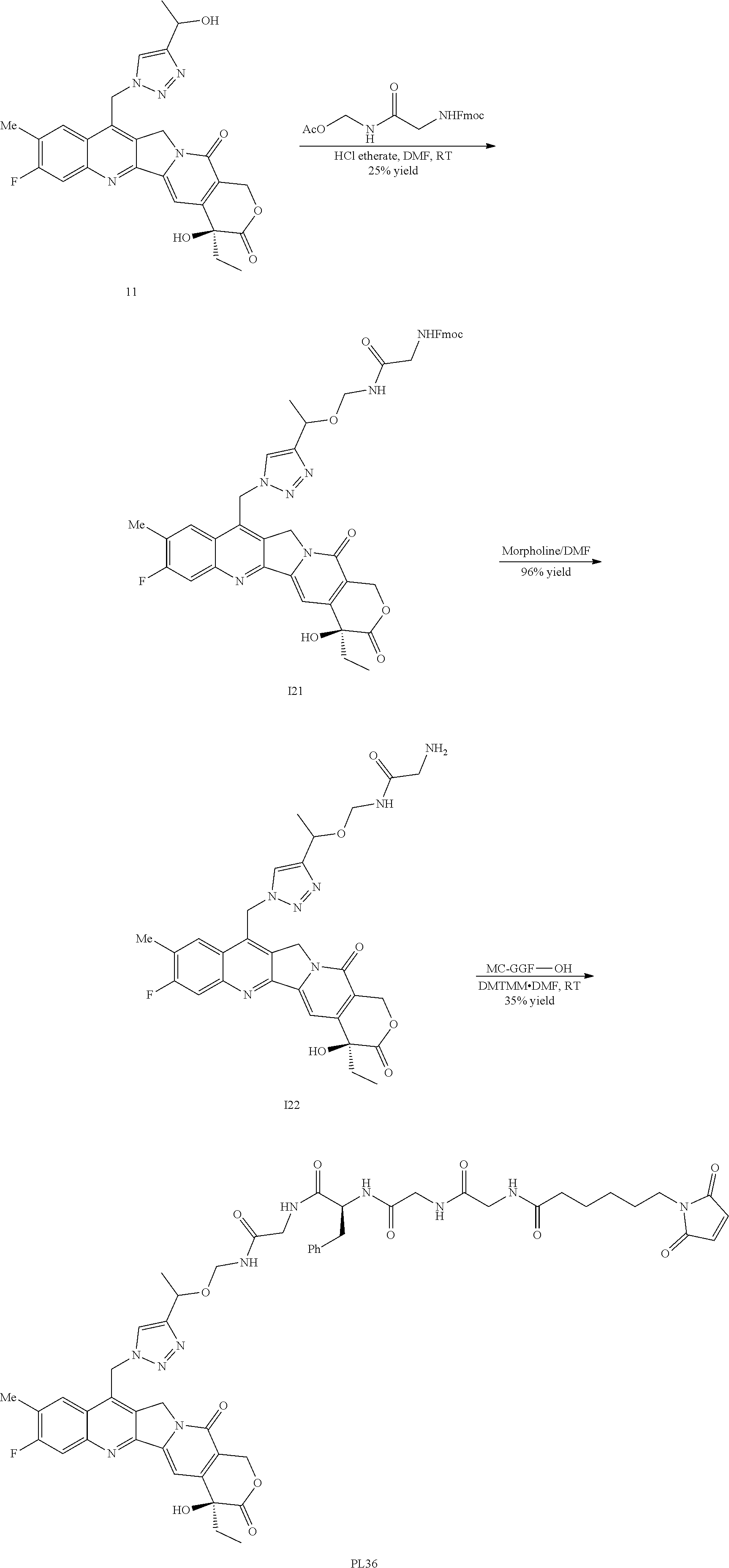

To an oven dried vial was added I1 (28 mg, 0.054 mmol, 1 eq) and OAc—CH₂—NH-Gly-Fmoc (20 mg, 0.054 mmol, 1 eq) and suspended in Anhydrous DMF (0.77 mL) with a brief sonication under Ar. To the vial, 1M HCl etherate (0.089 mL, 0.065 mmol, 1.2 eq) was added dropwise. The vial was stirred for 1.5 h at room temperature and then OAc—CH₂—NH-Gly-Fmoc (20 mg, 0.054 mmol, 1 eq) was added (total eq=2 thus far). The vial was stirred for 1.5 h at room temperature and then OAc—CH₂—NH-Gly-Fmoc (20 mg, 0.054 mmol, 1 eq) was added (total eq=3 thus far). The vial was stirred for 1.5 h at room temperature and then OAc—CH₂—NH-Gly-Fmoc (20 mg, 0.054 mmol, 1 eq) was added (total eq=4 thus far). The vial was stirred for 1.5 h at room temperature and then OAc—CH₂—NH-Gly-Fmoc (20 mg, 0.054 mmol, 1 eq) was added (total eq=5 thus far). The vial was stirred for 16 h at room temperature. LCMS indicated that the reaction had stalled, and the reaction was purified via reverse phase (Teledyne 50 g C18Aq with (5% ACN+0.05% FA)/(H₂O+0.05% FA) to 95% (ACN+0.05% FA)/(H₂O+0.05% FA) gradient). The product was recovered by lyophilization to obtain 121 (11.1 mg, 25% yield) as a white solid. (ESI) m/z: [M+H]+=814.49.

To an oven dried vial was added I21 (11 mg, 0.014 mmol, 1 eq) and dissolved in (1:4) (Morpholine:DMF) (0.195 mL) and stirred at room temperature under Ar. LCMS at 60 minutes indicated that 121 was consumed. The crude reaction was precipitated with diethyl ether (2 mL) and the solid was obtained by centrifugation. The solid was washed with diethyl ether (1×) (2 mL) and the solid was obtained by centrifugation. This obtained solid was redissolved in (1:1) (H₂O:ACN) and lyophilized to give 122 (7.8 mg, 96% yield) as a white solid. (ESI) m/z: [M+H]+=592.35.

To an oven dried vial was added I22 (7.8 mg, 0.013 mmol, 1 eq) and MC-GGF-OH (7.8 mg, 0.016 mmol, 1.25 eq) under Ar. Anhydrous DMF (0.41 mL) was added to the vial to yield a solution. DMTMM (7.3 mg, 0.026 mmol, 2 eq) was added to the vial and briefly sonicated to yield a suspension. The vial was stirred at room temperature for 0.5 h whereupon the LCMS indicated I22 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL36 (4.8 mg, 35% yield) as a white solid. (ESI) m/z: [M+H]+=1046.94.

### Example 44: Synthesis of PL37

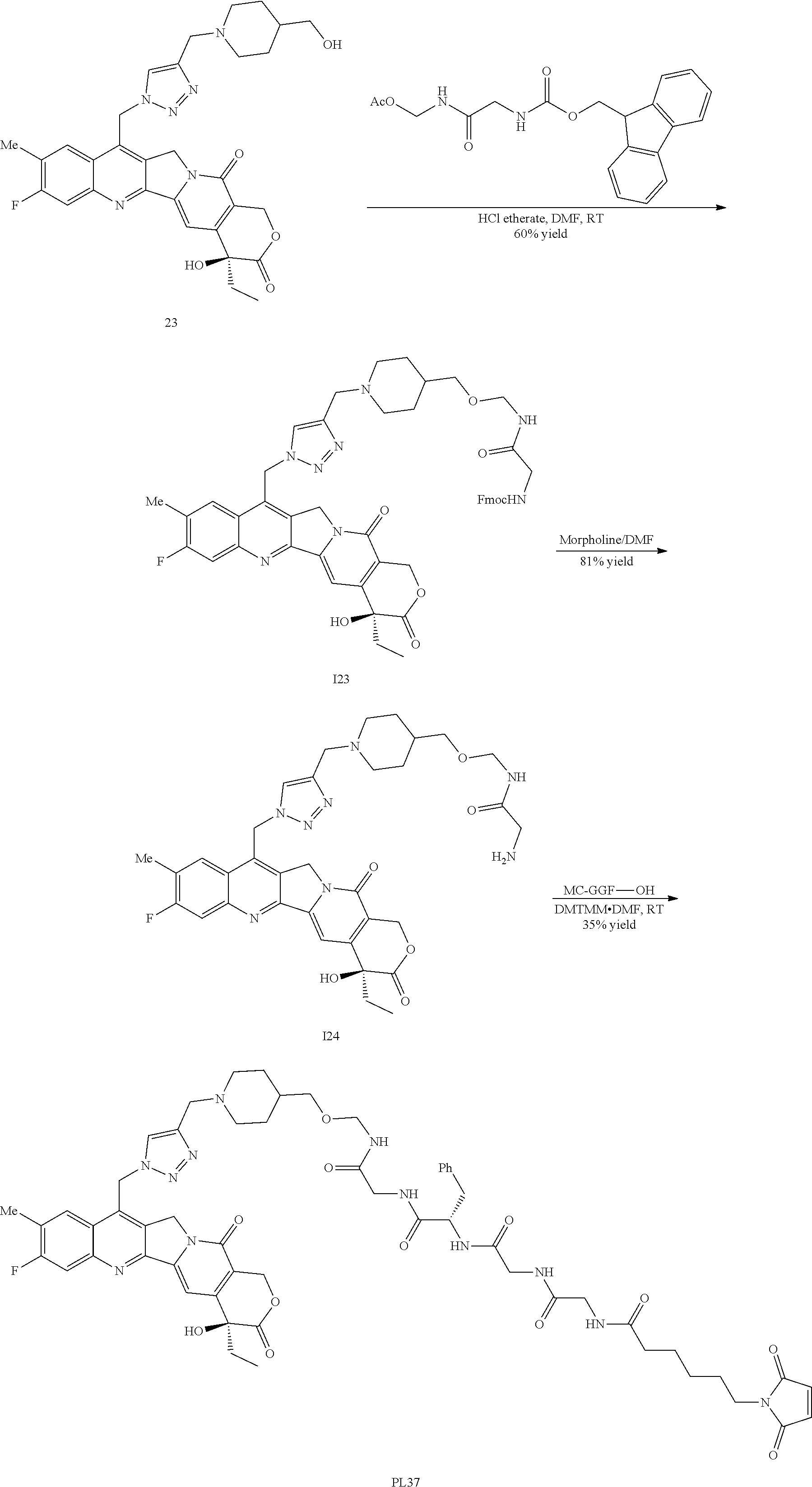

To an oven dried vial was added 23 (19 mg, 0.032 mmol, 1 eq) and OAc—CH₂—NH-Gly-Fmoc (12 mg, 0.032 mmol, 1 eq) and suspended in Anhydrous DMF (0.45 mL) with a brief sonication under Ar. To the vial, 1M HCl etherate (0.061 mL, 0.038 mmol, 1.2 eq) was added dropwise. The vial was stirred for 1.5 h at room temperature and then OAc—CH₂—NH-Gly-Fmoc (12 mg, 0.032 mmol, 1 eq) was added (total eq=2 thus far). The vial was stirred for 1.5 h at room temperature and then OAc—CH₂—NH-Gly-Fmoc (12 mg, 0.032 mmol, 1 eq) was added (total eq=3 thus far). The vial was stirred for 1.5 h at room temperature and then OAc—CH₂—NH-Gly-Fmoc (12 mg, 0.032 mmol, 1 eq) was added (total eq=4 thus far). The vial was stirred for 1.5 h at room temperature and then OAc—CH₂—NH-Gly-Fmoc (12 mg, 0.032 mmol, 1 eq) was added (total eq=5 thus far). The vial was stirred for 16 h at room temperature. LCMS indicated that the reaction had stalled, and the reaction was purified via reverse phase (Teledyne 50 g C18Aq with (5% ACN+0.05% FA)/(H₂O+0.05% FA) to 95% (ACN+0.05% FA)/(H₂O+0.05% FA) gradient). The product was recovered by lyophilization to obtain 123 (17.2 mg, 60% yield) as a white solid. (ESI) m/z: [M+H]+=897.38.

To an oven dried vial was added I23 (17 mg, 0.019 mmol, 1 eq) and dissolved in (1:4) (Morpholine:DMF) (0.274 mL) and stirred at room temperature under Ar. LCMS at 60 minutes indicated that 123 was consumed. The crude reaction was precipitated with diethyl ether (3 mL) and the solid was obtained by centrifugation. The solid was washed with diethyl ether (1×) (3 mL) and the solid was obtained by centrifugation. This obtained solid was redissolved in (1:1) (H₂O:ACN) and lyophilized to give 124 (10.5 mg, 81% yield) as a white solid. (ESI) m/z: [M+H]+=675.28.

To an oven dried vial was added I24 (11 mg, 0.016 mmol, 1 eq) and MC-GGF-OH (9.2 mg, 0.019 mmol, 1.25 eq) under Ar. Anhydrous DMF (0.49 mL) was added to the vial to yield a solution. DMTMM (8.6 mg, 0.031 mmol, 2 eq) was added to the vial and briefly sonicated to yield a suspension. The vial was stirred at room temperature for 0.5 h whereupon the LCMS indicated I24 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL37 (6.1 mg, 35% yield) as a white solid. (ESI) m/z: [M+H]+=1129.48.

### Example 45: Synthesis of PL38

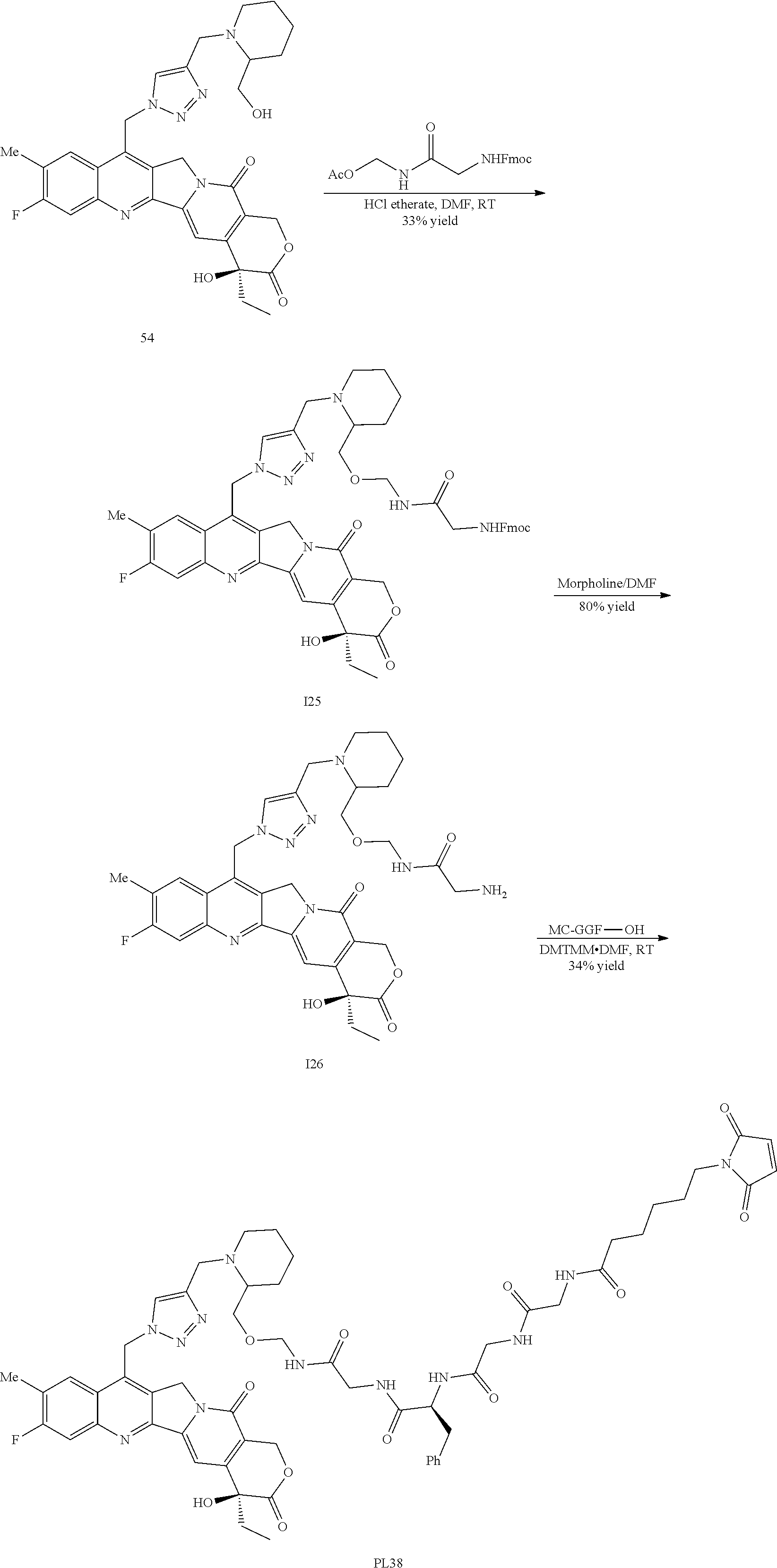

To an oven dried vial was added 54 (12 mg, 0.020 mmol, 1 eq) and OAc—CH₂—NH-Gly-Fmoc (7.3 mg, 0.020 mmol, 1 eq) and suspended in Anhydrous DMF (0.28 mL) with a brief sonication under Ar. To the vial, 1M HCl etherate (0.027 mL, 0.158 mmol, 8 eq) was added dropwise. The vial was stirred for 1.5 h at room temperature and then OAc—CH₂—NH-Gly-Fmoc (7.3 mg, 0.020 mmol, 1 eq) was added (total eq=2 thus far). The vial was stirred for 1.5 h at room temperature and then OAc—CH₂—NH-Gly-Fmoc (7.3 mg, 0.020 mmol, 1 eq) was added (total eq=3 thus far). The vial was stirred for 1.5 h at room temperature and then OAc—CH₂—NH-Gly-Fmoc (7.3 mg, 0.020 mmol, 1 eq) was added (total eq=4 thus far). The vial was stirred for 1.5 h at room temperature and then OAc—CH₂—NH-Gly-Fmoc (7.3 mg, 0.020 mmol, 1 eq) was added (total eq=5 thus far). The vial was stirred for 16 h at room temperature. LCMS indicated that the reaction had stalled, and the reaction was purified via reverse phase (Teledyne 50 g C18Aq with (5% ACN+0.05% FA)/(H₂O+0.05% FA) to 95% (ACN+0.05% FA)/(H₂O+0.05% FA) gradient). The product was recovered by lyophilization to obtain 125 (5.9 mg, 33% yield) as a white solid. (ESI) m/z: [M+H]+=897.41.

To an oven dried vial was added I25 (6 mg, 0.007 mmol, 1 eq) and dissolved in (1:4) (Morpholine:DMF) (0.094 mL) and stirred at room temperature under Ar. LCMS at 60 minutes indicated that 125 was consumed. The crude reaction was precipitated with diethyl ether (1 mL) and the solid was obtained by centrifugation. The solid was washed with diethyl ether (1×) (1 mL) and the solid was obtained by centrifugation. This obtained solid was redissolved in (1:1) (H₂O:ACN) and lyophilized to give 126 (3.5 mg, 80% yield) as a white solid. (ESI) m/z: [M+H]+=675.31.

To an oven dried vial was added I26 (4 mg, 0.005 mmol, 1 eq) and MC-GGF-OH (3.1 mg, 0.006 mmol, 1.25 eq) under Ar. Anhydrous DMF (0.16 mL) was added to the vial to yield a solution. DMTMM (3 mg, 0.010 mmol, 2 eq) was added to the vial and briefly sonicated to yield a suspension. The vial was stirred at room temperature for 0.5 h whereupon the LCMS indicated I26 was consumed. The crude reaction was purified via reverse phase (Teledyne Gemini (5 μm C18 110 Å 150×30 mm) with (5% ACN)/(H₂O) to 95% (ACN)/(H₂O) gradient). The product was recovered by lyophilization to obtain PL38 (2 mg, 34% yield) as a white solid. (ESI) m/z: [M+H]+=1129.51.

### Example 46: Synthesis of PL39

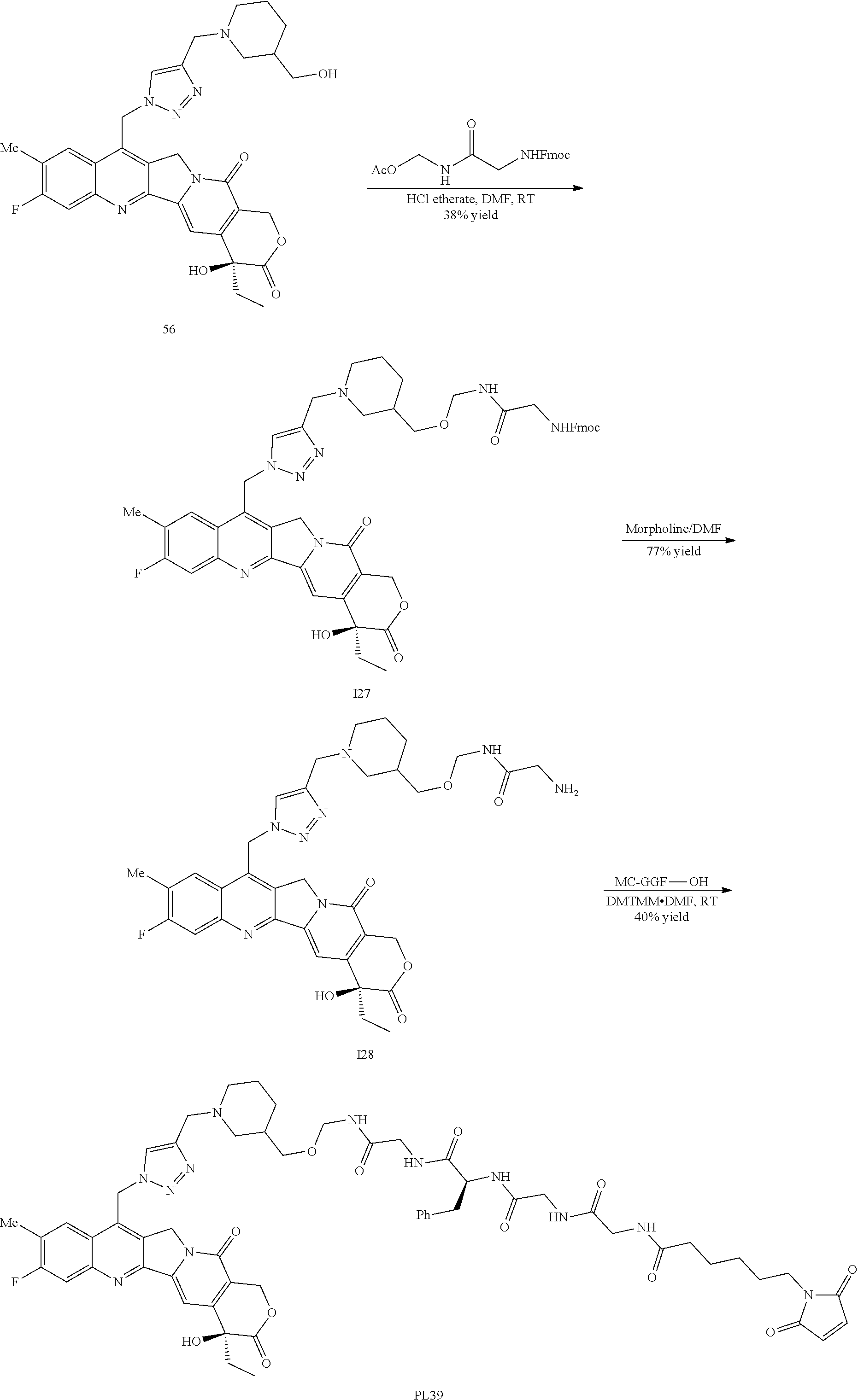

To an oven dried vial was added 56 (16 mg, 0.028 mmol, 1 eq) and OAc—CH₂—NH-Gly-Fmoc (10 mg, 0.028 mmol, 1 eq) and suspended in Anhydrous DMF (0.39 mL) with a brief sonication under Ar. To the vial, 1M HCl etherate (0.038 mL, 0.222 mmol, 8 eq) was added dropwise. The vial was stirred for 1.5 h at room temperature and then OAc—CH₂—NH-Gly-Fmoc (10 mg, 0.028 mmol, 1 eq) was added (total eq=2 thus far). The vial was stirred for 1.5 h at room temperature and then OAc—CH₂—NH-Gly-Fmoc (10 mg, 0.028 mmol, 1 eq) was added (total eq=3 thus far). The vial was stirred for 1.5 h at room temperature and then OAc—CH₂—NH-Gly-Fmoc (10 mg, 0.028 mmol, 1 eq) was added (total eq=4 thus far). The vial was stirred for 1.5 h at room temperature and then OAc—CH₂—NH-Gly-Fmoc (10 mg, 0.028 mmol, 1 eq) was added (total eq=5 thus far). The vial was stirred for 16 h at room temperature. LCMS indicated that the reaction had stalled, and the reaction was purified via reverse phase (Teledyne 50 g C18Aq with (5% ACN+0.05% FA)/(H₂O+0.05% FA) to 95% (ACN+0.05% FA)/(H₂O+0.05% FA) gradient). The product was recovered by lyophilization to obtain 127 (9.3 mg, 38% yield) as a white solid. (ESI) m/z: [M+H]+=897.41.

To an oven dried vial was added I27 (9 mg, 0.010 mmol, 1 eq) and dissolved in (1:4) (Morpholine:DMF) (0.148 mL) and stirred at room temperature under Ar. LCMS at 60 minutes indicated that I27 was consumed. The crude reaction was precipitated with diethyl ether (2 mL) and the solid was obtained by centrifugation. The solid was washed with diethyl ether (1×) (2 mL) and the solid was obtained by centrifugation. This obtained solid was redissolved in (1:1) (H₂O:ACN) and lyophilized to give 128 (5.4 mg, 77% yield) as a white solid. (ESI) m/z: [M+H]+=675.38.

To an oven dried vial was added I28 (5 mg, 0.008 mmol, 1 eq) and MC-GGF-OH (4.7 mg, 0.010 mmol, 1.25 eq) under Ar. Anhydrous DMF (0.25 mL) was added to the vial to yield a solution. DMTMM (4.4 mg, 0.016 mmol, 2 eq) was added to the vial and briefly sonicated to yield a suspension. The vial was stirred at room temperature for 0.5 h whereupon the LCMS indicated I28 was consumed. The crude reaction was purified via reverse phase (Teledyne Gemini (5 μm C18 110 Å, 150×30 mm) with (5% ACN)/(H₂O) to 95% (ACN)/(H₂O) gradient). The product was recovered by lyophilization to obtain PL39 (3.6 mg, 4000 yield) as a white solid. (ESI) m/z: [M+H]+=1129.49.

TABLE 03
Payload Linker Compounds (PLs)

| Cmpd | Structure |
|---|---|
| PL1 | 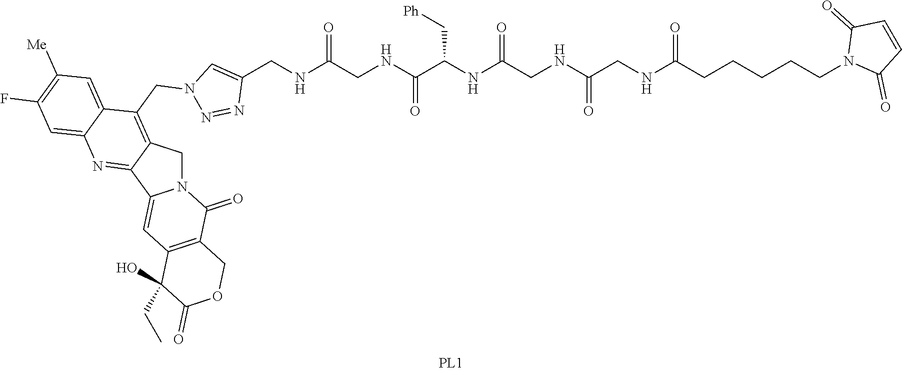 [SMILES: CC[C@@]1(O)C(=O)OCc2c1cc1n(c2=O)Cc2c-1nc1cc(F)c(C)cc1c2Cn1cc(CNC(=O)CNC(=O)[C@H](Cc2ccccc2)NC(=O)CNC(=O)CNC(=O)CCCCCN2C(=O)C=CC2=O)nn1] |
| PL2 | 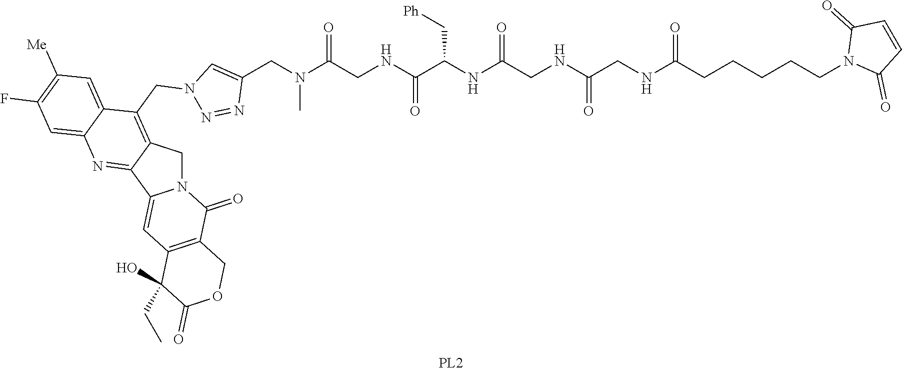 [SMILES: CC[C@@]1(O)C(=O)OCc2c1cc1n(c2=O)Cc2c-1nc1cc(F)c(C)cc1c2Cn1cc(CN(C)C(=O)CNC(=O)[C@H](Cc2ccccc2)NC(=O)CNC(=O)CNC(=O)CCCCCN2C(=O)C=CC2=O)nn1] |
| PL3 | 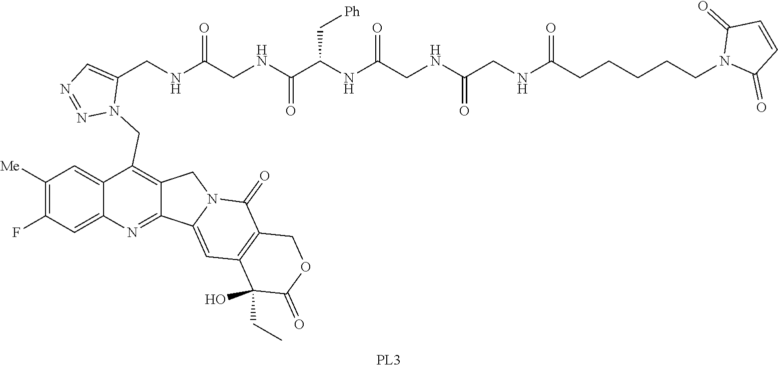 [SMILES: CC[C@@]1(O)C(=O)OCc2c1cc1n(c2=O)Cc2c-1nc1cc(F)c(C)cc1c2Cn1nncc1CNC(=O)CNC(=O)[C@H](Cc1ccccc1)NC(=O)CNC(=O)CNC(=O)CCCCCN1C(=O)C=CC1=O] |
| PL4 | 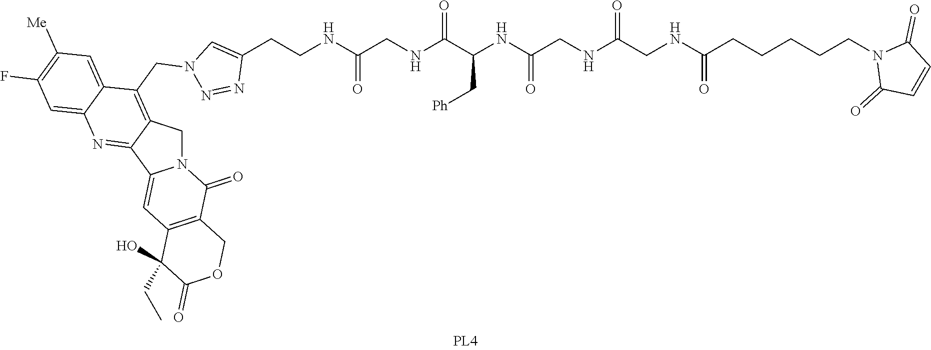 [SMILES: CC[C@@]1(O)C(=O)OCc2c1cc1n(c2=O)Cc2c-1nc1cc(F)c(C)cc1c2Cn1cc(CCNC(=O)CNC(=O)[C@H](Cc2ccccc2)NC(=O)CNC(=O)CNC(=O)CCCCCN2C(=O)C=CC2=O)nn1] |
| PL5 | 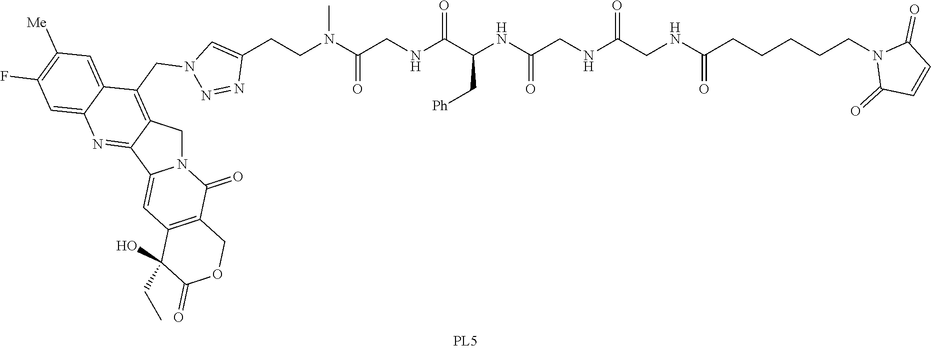 [SMILES: CC[C@@]1(O)C(=O)OCc2c1cc1n(c2=O)Cc2c-1nc1cc(F)c(C)cc1c2Cn1cc(CCN(C)C(=O)CNC(=O)[C@H](Cc2ccccc2)NC(=O)CNC(=O)CNC(=O)CCCCCN2C(=O)C=CC2=O)nn1] |
| PL6 | 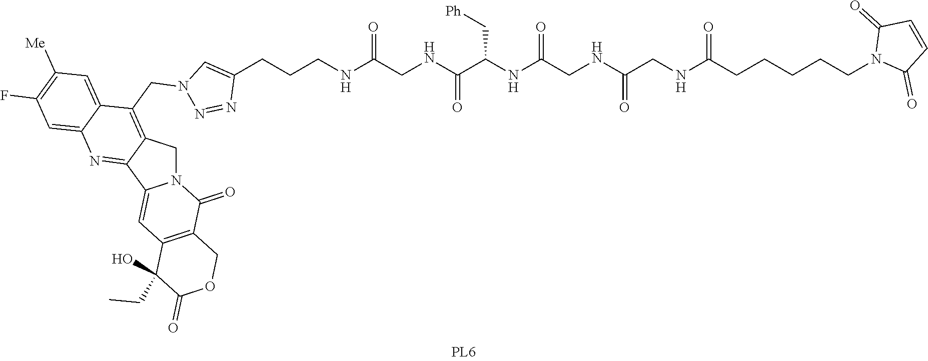 [SMILES: CC[C@@]1(O)C(=O)OCc2c1cc1n(c2=O)Cc2c-1nc1cc(F)c(C)cc1c2Cn1cc(CCCNC(=O)CNC(=O)[C@H](Cc2ccccc2)NC(=O)CNC(=O)CNC(=O)CCCCCN2C(=O)C=CC2=O)nn1] |
| PL7 | 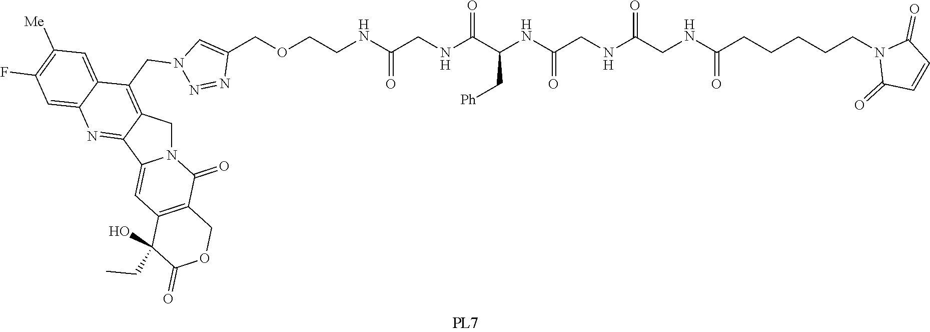 [SMILES: CC[C@@]1(O)C(=O)OCc2c1cc1n(c2=O)Cc2c-1nc1cc(F)c(C)cc1c2Cn1cc(COCCNC(=O)CNC(=O)[C@H](Cc2ccccc2)NC(=O)CNC(=O)CNC(=O)CCCCCN2C(=O)C=CC2=O)nn1] |
| PL8 | 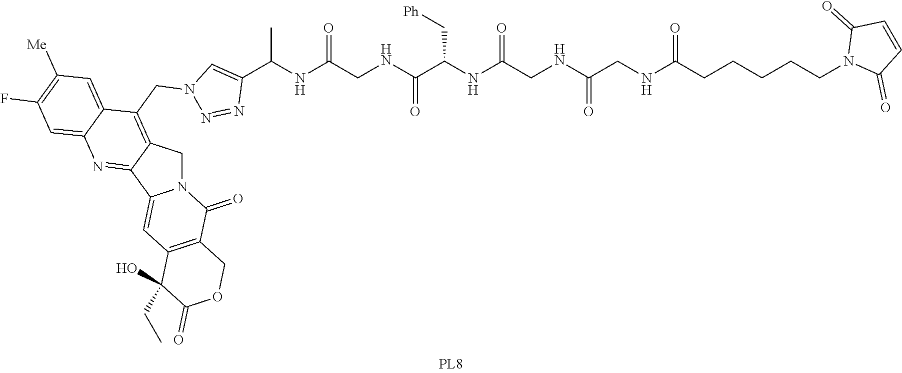 [SMILES: CC[C@@]1(O)C(=O)OCc2c1cc1n(c2=O)Cc2c-1nc1cc(F)c(C)cc1c2Cn1cc(C(C)NC(=O)CNC(=O)[C@H](Cc2ccccc2)NC(=O)CNC(=O)CNC(=O)CCCCCN2C(=O)C=CC2=O)nn1] |
| PL9 | 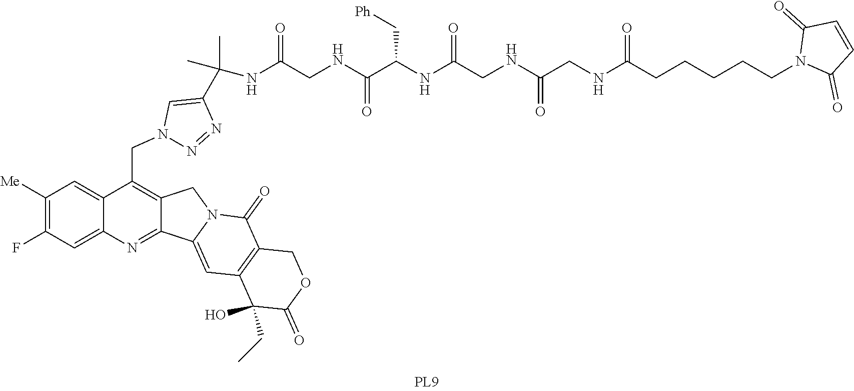 [SMILES: CC[C@@]1(O)C(=O)OCc2c1cc1n(c2=O)Cc2c-1nc1cc(F)c(C)cc1c2Cn1cc(C(C)(C)NC(=O)CNC(=O)[C@H](Cc2ccccc2)NC(=O)CNC(=O)CNC(=O)CCCCCN2C(=O)C=CC2=O)nn1] |
| PL10 | 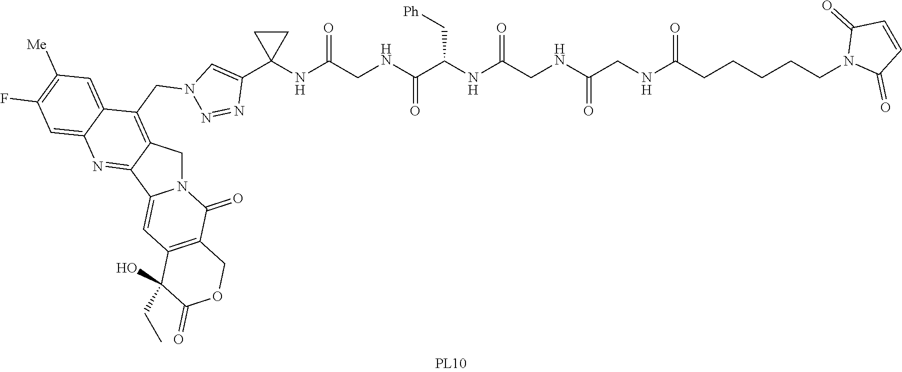 [SMILES: CC[C@@]1(O)C(=O)OCc2c1cc1n(c2=O)Cc2c-1nc1cc(F)c(C)cc1c2Cn1cc(C2(NC(=O)CNC(=O)[C@H](Cc3ccccc3)NC(=O)CNC(=O)CNC(=O)CCCCCN3C(=O)C=CC3=O)CC2)nn1] |
| PL11 | 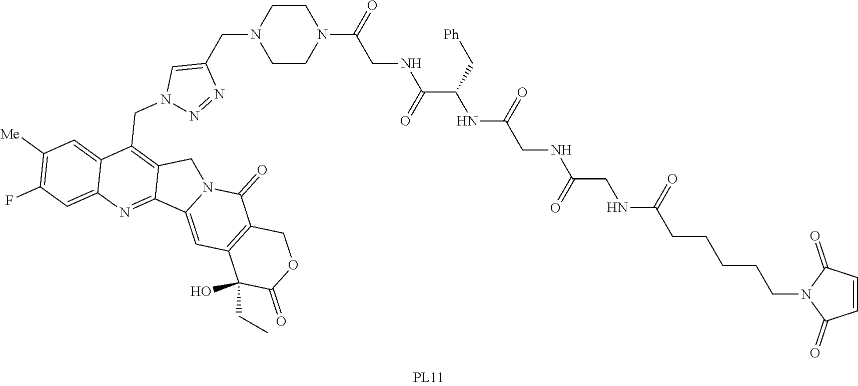 [SMILES: CC[C@@]1(O)C(=O)OCc2c1cc1n(c2=O)Cc2c-1nc1cc(F)c(C)cc1c2Cn1cc(CN2CCN(C(=O)CNC(=O)[C@H](Cc3ccccc3)NC(=O)CNC(=O)CNC(=O)CCCCCN3C(=O)C=CC3=O)CC2)nn1] |
| PL12 | 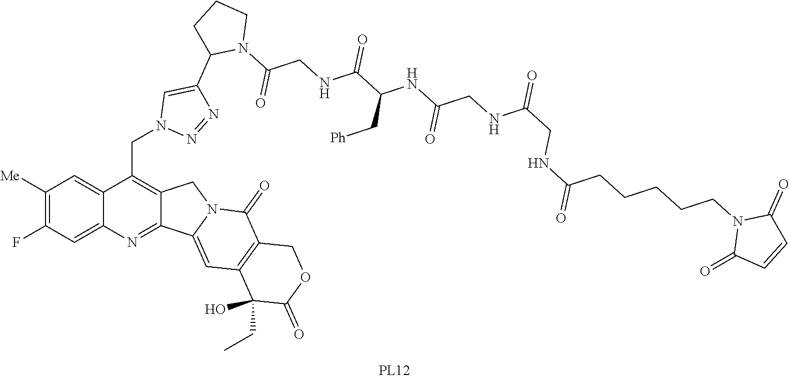 [SMILES: CC[C@@]1(O)C(=O)OCc2c1cc1n(c2=O)Cc2c-1nc1cc(F)c(C)cc1c2Cn1cc(C2CCCN2C(=O)CNC(=O)[C@H](Cc2ccccc2)NC(=O)CNC(=O)CNC(=O)CCCCCN2C(=O)C=CC2=O)nn1] |
| PL13 | 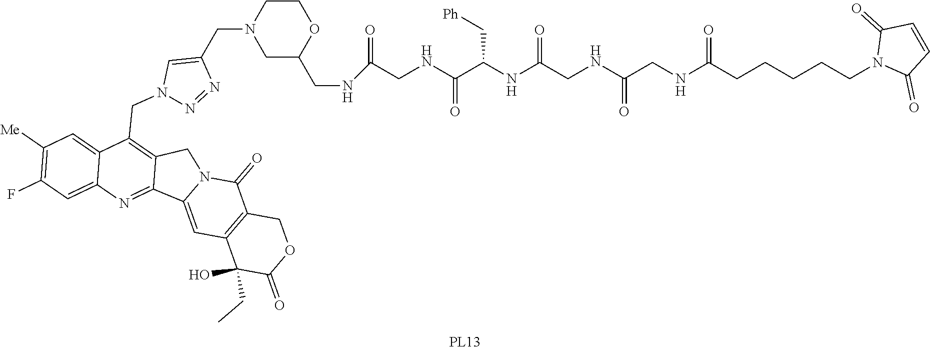 [SMILES: CC[C@@]1(O)C(=O)OCc2c1cc1n(c2=O)Cc2c-1nc1cc(F)c(C)cc1c2Cn1cc(CN2CCOC(CNC(=O)CNC(=O)[C@H](Cc3ccccc3)NC(=O)CNC(=O)CNC(=O)CCCCCN3C(=O)C=CC3=O)C2)nn1] |
| PL14 | 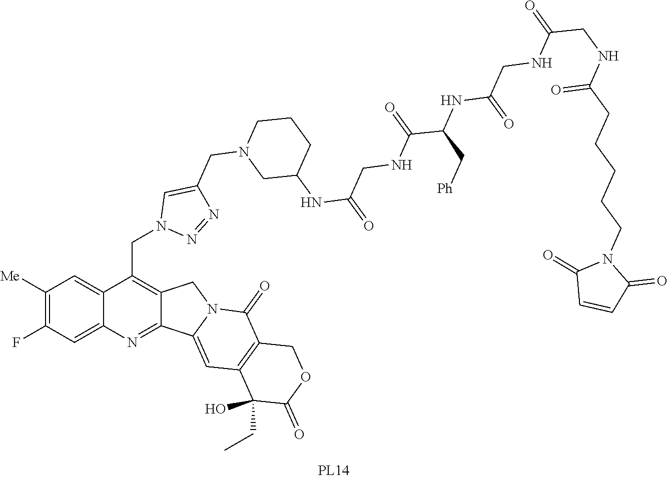 [SMILES: CC[C@@]1(O)C(=O)OCc2c1cc1n(c2=O)Cc2c-1nc1cc(F)c(C)cc1c2Cn1cc(CN2CCCC(NC(=O)CNC(=O)[C@H](Cc3ccccc3)NC(=O)CNC(=O)CNC(=O)CCCCCN3C(=O)C=CC3=O)C2)nn1] |
| PL15 | 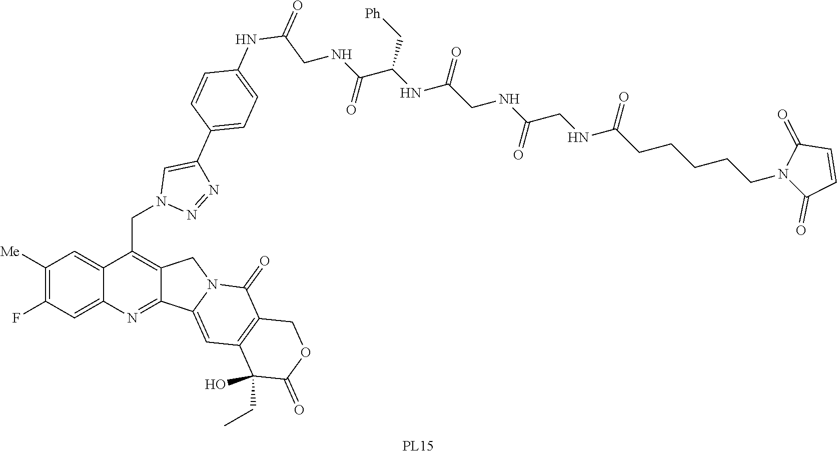 [SMILES: CC[C@@]1(O)C(=O)OCc2c1cc1n(c2=O)Cc2c-1nc1cc(F)c(C)cc1c2Cn1cc(-c2ccc(NC(=O)CNC(=O)[C@H](Cc3ccccc3)NC(=O)CNC(=O)CNC(=O)CCCCCN3C(=O)C=CC3=O)cc2)nn1] |
| PL16 | 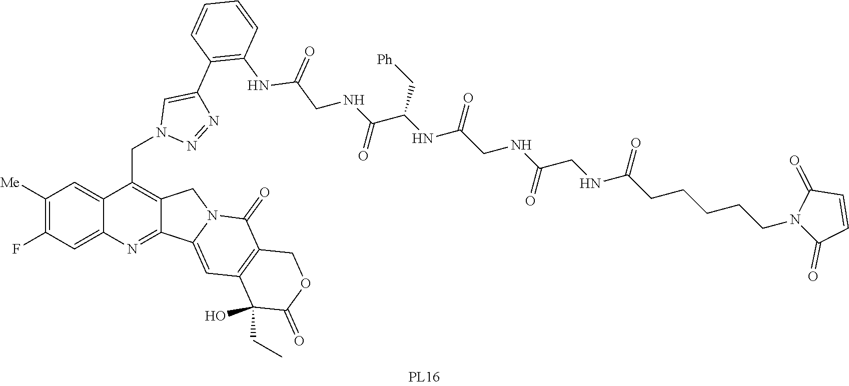 [SMILES: CC[C@@]1(O)C(=O)OCc2c1cc1n(c2=O)Cc2c-1nc1cc(F)c(C)cc1c2Cn1cc(-c2ccccc2NC(=O)CNC(=O)[C@H](Cc2ccccc2)NC(=O)CNC(=O)CNC(=O)CCCCCN2C(=O)C=CC2=O)nn1] |
| PL17 | 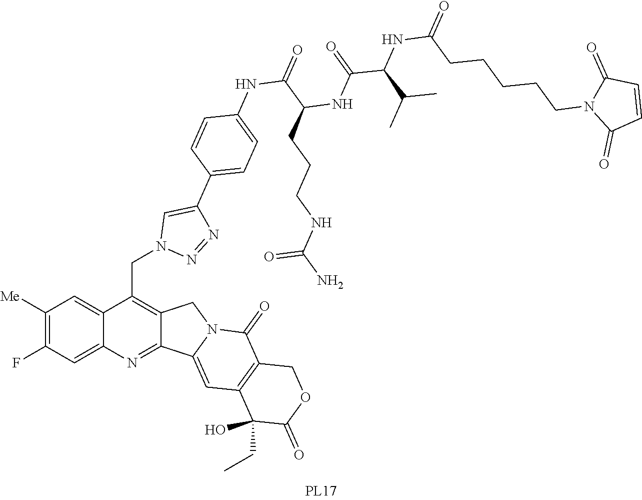 [SMILES: CC[C@@]1(O)C(=O)OCc2c1cc1n(c2=O)Cc2c-1nc1cc(F)c(C)cc1c2Cn1cc(-c2ccc(NC(=O)[C@H](CCCNC(N)=O)NC(=O)[C@@H](NC(=O)CCCCCN3C(=O)C=CC3=O)C(C)C)cc2)nn1] |
| PL18 | 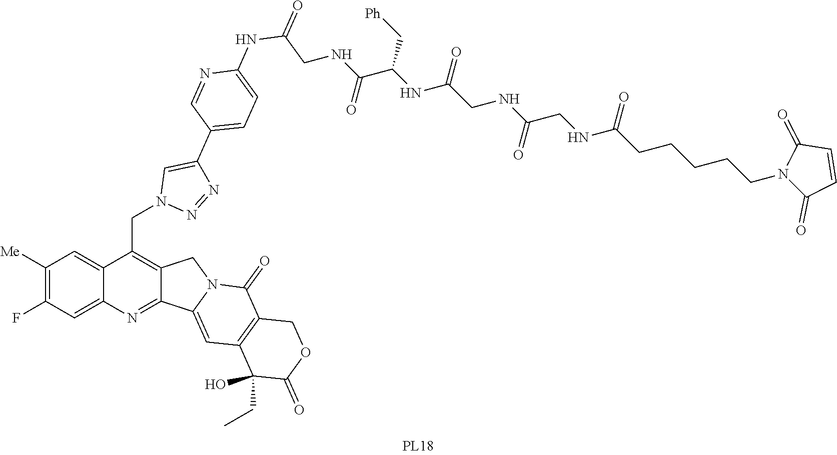 [SMILES: CC[C@@]1(O)C(=O)OCc2c1cc1n(c2=O)Cc2c-1nc1cc(F)c(C)cc1c2Cn1cc(-c2ccc(NC(=O)CNC(=O)[C@H](Cc3ccccc3)NC(=O)CNC(=O)CNC(=O)CCCCCN3C(=O)C=CC3=O)nc2)nn1] |
| PL19 | 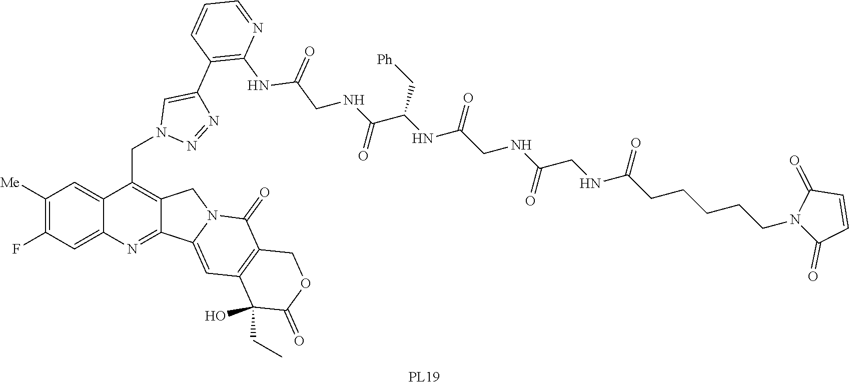 [SMILES: CC[C@@]1(O)C(=O)OCc2c1cc1n(c2=O)Cc2c-1nc1cc(F)c(C)cc1c2Cn1cc(-c2cccnc2NC(=O)CNC(=O)[C@H](Cc2ccccc2)NC(=O)CNC(=O)CNC(=O)CCCCCN2C(=O)C=CC2=O)nn1] |
| PL20 | 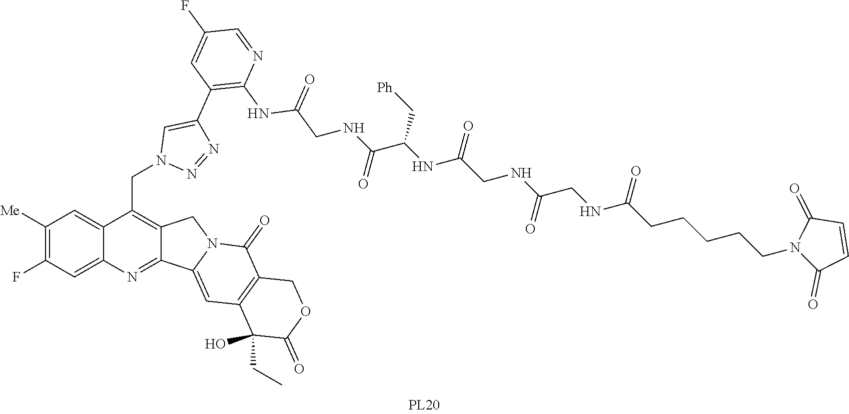 [SMILES: CC[C@@]1(O)C(=O)OCc2c1cc1n(c2=O)Cc2c-1nc1cc(F)c(C)cc1c2Cn1cc(-c2cc(F)cnc2NC(=O)CNC(=O)[C@H](Cc2ccccc2)NC(=O)CNC(=O)CNC(=O)CCCCCN2C(=O)C=CC2=O)nn1] |
| PL21 | 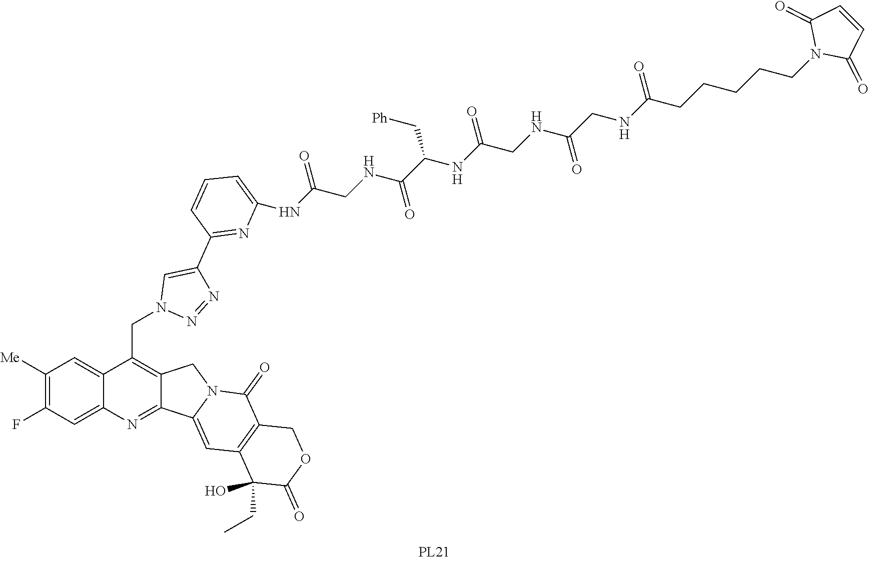 [SMILES: CC[C@@]1(O)C(=O)OCc2c1cc1n(c2=O)Cc2c-1nc1cc(F)c(C)cc1c2Cn1cc(-c2cccc(NC(=O)CNC(=O)[C@H](Cc3ccccc3)NC(=O)CNC(=O)CNC(=O)CCCCCN3C(=O)C=CC3=O)n2)nn1] |
| PL22 | 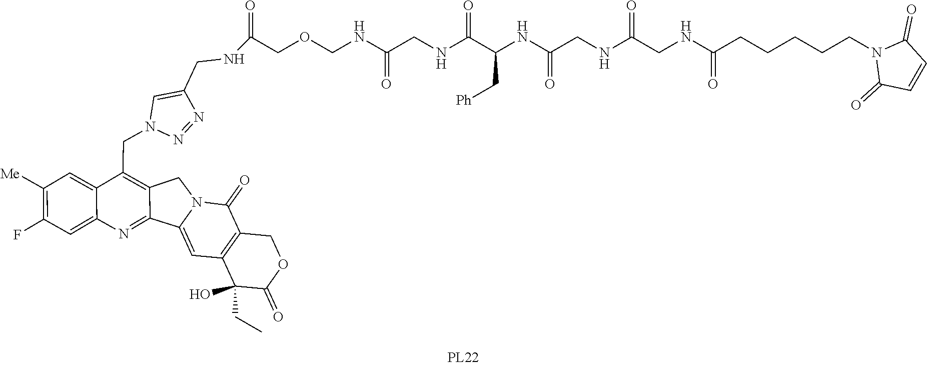 [SMILES: CC[C@@]1(O)C(=O)OCc2c1cc1n(c2=O)Cc2c-1nc1cc(F)c(C)cc1c2Cn1cc(CNC(=O)COCNC(=O)CNC(=O)[C@H](Cc2ccccc2)NC(=O)CNC(=O)CNC(=O)CCCCCN2C(=O)C=CC2=O)nn1] |
| PL23 | 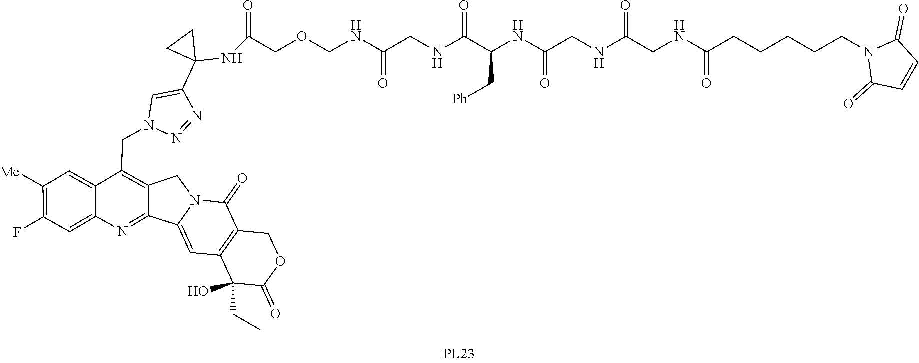 [SMILES: CC[C@@]1(O)C(=O)OCc2c1cc1n(c2=O)Cc2c-1nc1cc(F)c(C)cc1c2Cn1cc(C2(NC(=O)COCNC(=O)CNC(=O)[C@H](Cc3ccccc3)NC(=O)CNC(=O)CNC(=O)CCCCCN3C(=O)C=CC3=O)CC2)nn1] |
| PL24 | 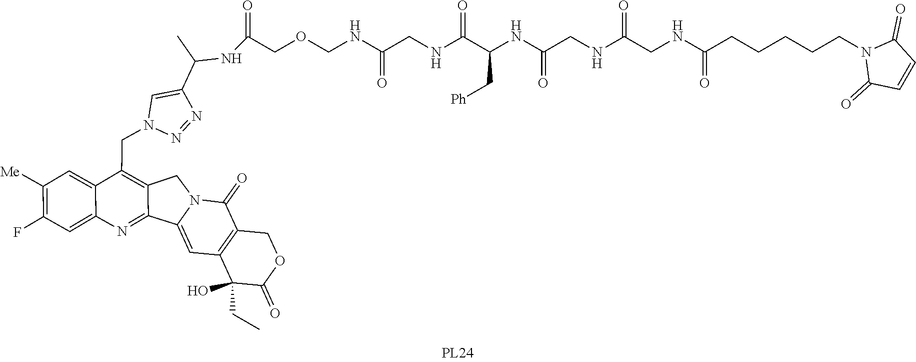 [SMILES: CC[C@@]1(O)C(=O)OCc2c1cc1n(c2=O)Cc2c-1nc1cc(F)c(C)cc1c2Cn1cc(C(C)NC(=O)COCNC(=O)CNC(=O)[C@H](Cc2ccccc2)NC(=O)CNC(=O)CNC(=O)CCCCCN2C(=O)C=CC2=O)nn1] |
| PL25 | 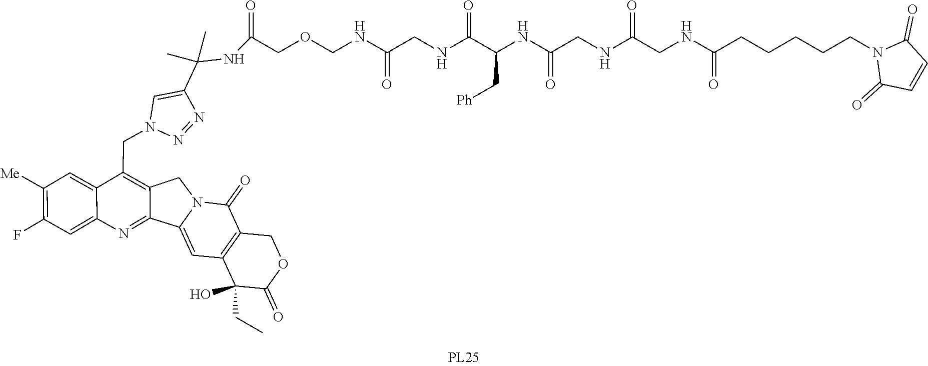 [SMILES: CC[C@@]1(O)C(=O)OCc2c1cc1n(c2=O)Cc2c-1nc1cc(F)c(C)cc1c2Cn1cc(C(C)(C)NC(=O)COCNC(=O)CNC(=O)[C@H](Cc2ccccc2)NC(=O)CNC(=O)CNC(=O)CCCCCN2C(=O)C=CC2=O)nn1] |
| PL26 | 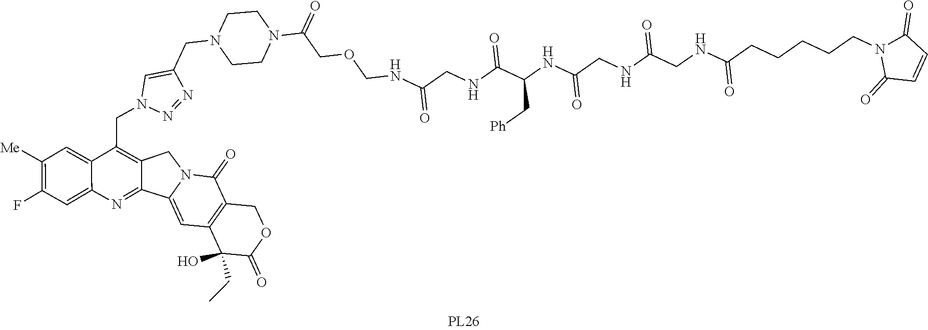 [SMILES: CC[C@@]1(O)C(=O)OCc2c1cc1n(c2=O)Cc2c-1nc1cc(F)c(C)cc1c2Cn1cc(CN2CCN(C(=O)COCNC(=O)CNC(=O)[C@H](Cc3ccccc3)NC(=O)CNC(=O)CNC(=O)CCCCCN3C(=O)C=CC3=O)CC2)nn1] |
| PL27 | 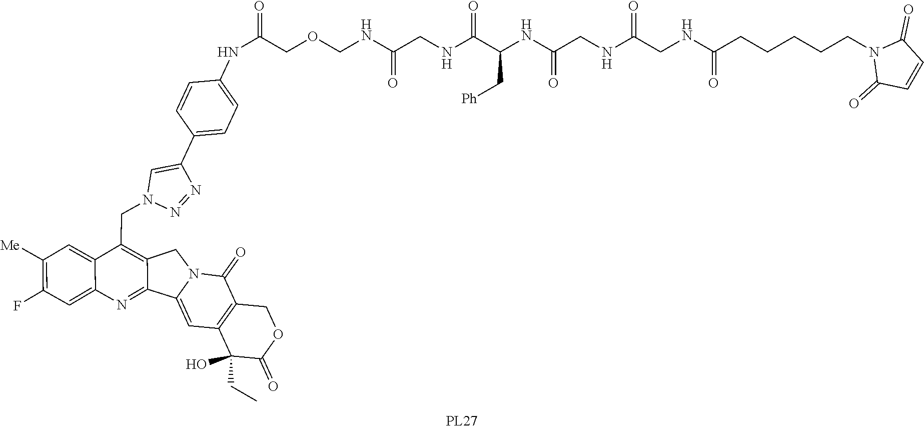 [SMILES: CC[C@@]1(O)C(=O)OCc2c1cc1n(c2=O)Cc2c-1nc1cc(F)c(C)cc1c2Cn1cc(-c2ccc(NC(=O)COCNC(=O)CNC(=O)[C@H](Cc3ccccc3)NC(=O)CNC(=O)CNC(=O)CCCCCN3C(=O)C=CC3=O)cc2)nn1] |
| PL28 | 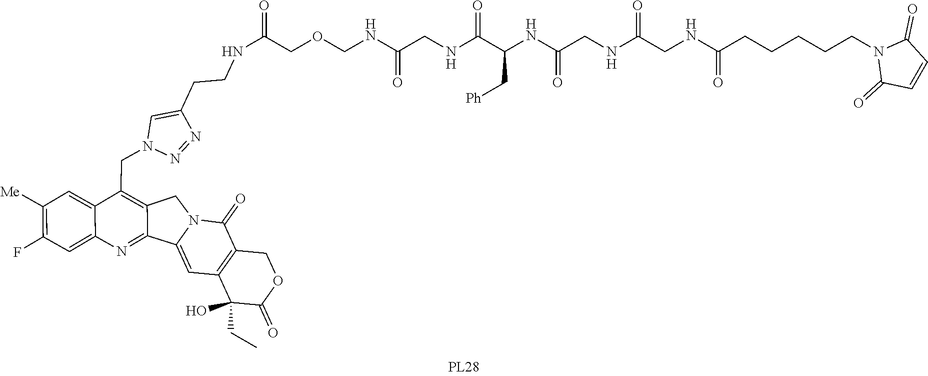 [SMILES: CC[C@@]1(O)C(=O)OCc2c1cc1n(c2=O)Cc2c-1nc1cc(F)c(C)cc1c2Cn1cc(CCNC(=O)COCNC(=O)CNC(=O)[C@H](Cc2ccccc2)NC(=O)CNC(=O)CNC(=O)CCCCCN2C(=O)C=CC2=O)nn1] |
| PL29 | 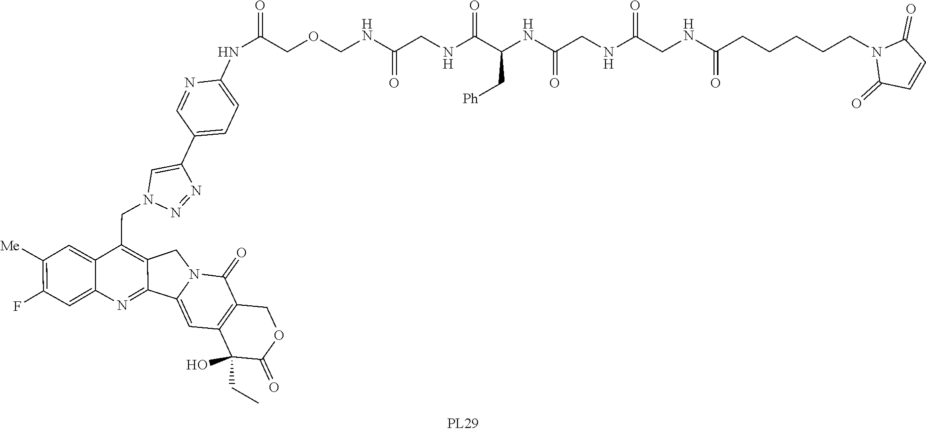 [SMILES: CC[C@@]1(O)C(=O)OCc2c1cc1n(c2=O)Cc2c-1nc1cc(F)c(C)cc1c2Cn1cc(-c2ccc(NC(=O)COCNC(=O)CNC(=O)[C@H](Cc3ccccc3)NC(=O)CNC(=O)CNC(=O)CCCCCN3C(=O)C=CC3=O)nc2)nn1] |
| PL30 | 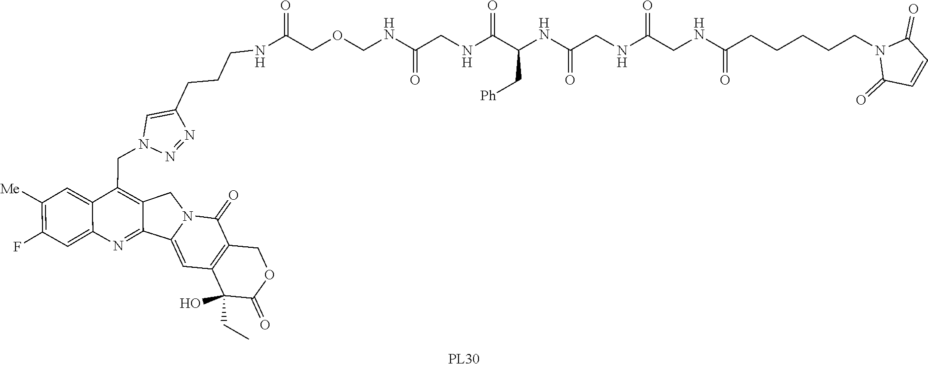 [SMILES: CC[C@@]1(O)C(=O)OCc2c1cc1n(c2=O)Cc2c-1nc1cc(F)c(C)cc1c2Cn1cc(CCCNC(=O)COCNC(=O)CNC(=O)[C@H](Cc2ccccc2)NC(=O)CNC(=O)CNC(=O)CCCCCN2C(=O)C=CC2=O)nn1] |
| PL31 | 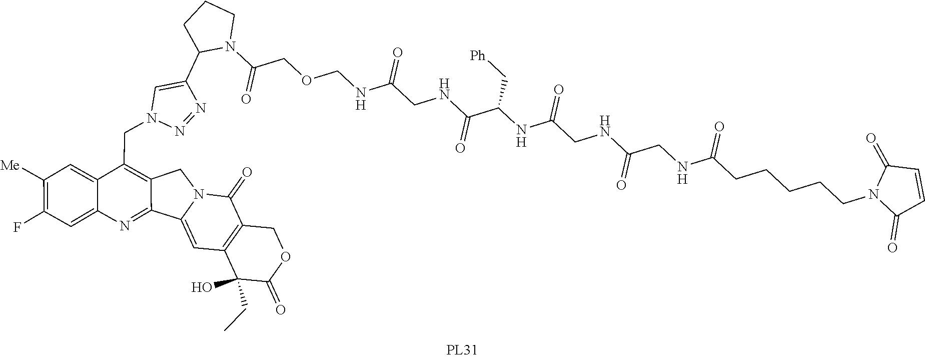 [SMILES: CC[C@@]1(O)C(=O)OCc2c1cc1n(c2=O)Cc2c-1nc1cc(F)c(C)cc1c2Cn1cc(C2CCCN2C(=O)COCNC(=O)CNC(=O)[C@H](Cc2ccccc2)NC(=O)CNC(=O)CNC(=O)CCCCCN2C(=O)C=CC2=O)nn1] |
| PL32 | 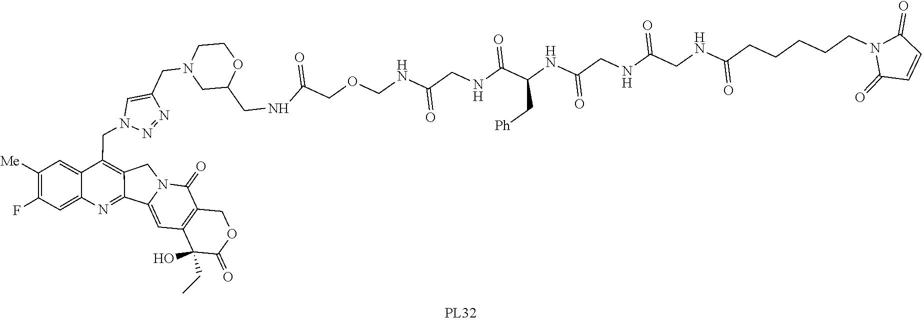 [SMILES: CC[C@@]1(O)C(=O)OCc2c1cc1n(c2=O)Cc2c-1nc1cc(F)c(C)cc1c2Cn1cc(CN2CCOC(CNC(=O)COCNC(=O)CNC(=O)[C@H](Cc3ccccc3)NC(=O)CNC(=O)CNC(=O)CCCCCN3C(=O)C=CC3=O)C2)nn1] |
| PL33 | 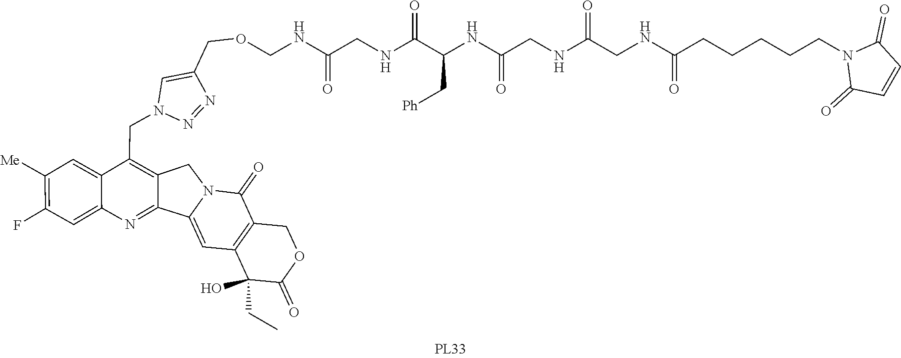 [SMILES: CC[C@@]1(O)C(=O)OCc2c1cc1n(c2=O)Cc2c-1nc1cc(F)c(C)cc1c2Cn1cc(COCNC(=O)CNC(=O)[C@H](Cc2ccccc2)NC(=O)CNC(=O)CNC(=O)CCCCCN2C(=O)C=CC2=O)nn1] |
| PL34 | 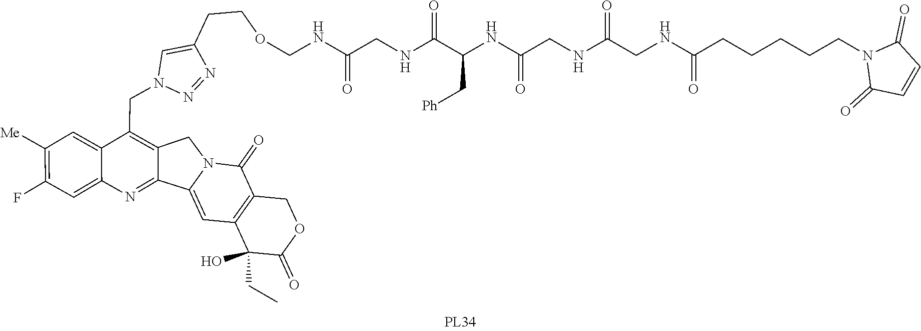 [SMILES: CC[C@@]1(O)C(=O)OCc2c1cc1n(c2=O)Cc2c-1nc1cc(F)c(C)cc1c2Cn1cc(CCOCNC(=O)CNC(=O)[C@H](Cc2ccccc2)NC(=O)CNC(=O)CNC(=O)CCCCCN2C(=O)C=CC2=O)nn1] |
| PL35 | 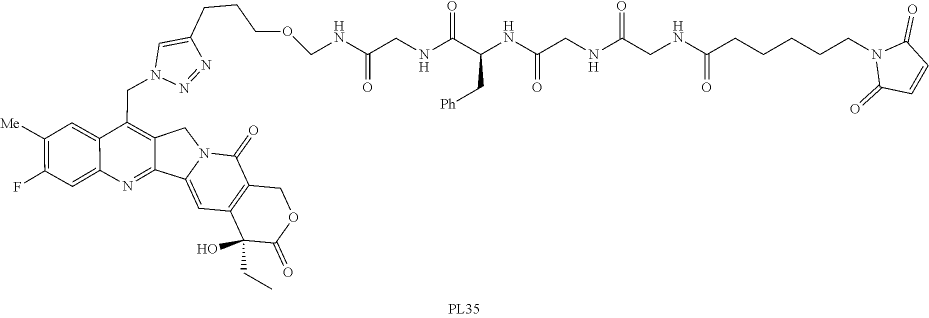 [SMILES: CC[C@@]1(O)C(=O)OCc2c1cc1n(c2=O)Cc2c-1nc1cc(F)c(C)cc1c2Cn1cc(CCCOCNC(=O)CNC(=O)[C@H](Cc2ccccc2)NC(=O)CNC(=O)CNC(=O)CCCCCN2C(=O)C=CC2=O)nn1] |
| PL36 | 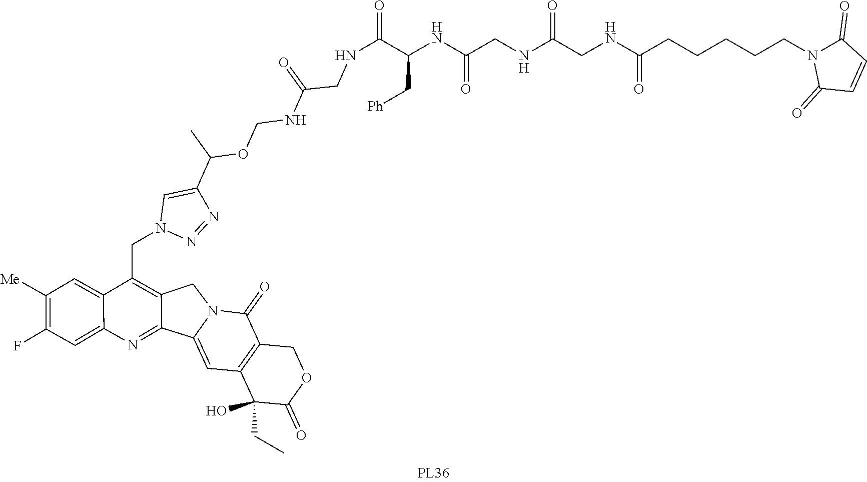 [SMILES: CC[C@@]1(O)C(=O)OCc2c1cc1n(c2=O)Cc2c-1nc1cc(F)c(C)cc1c2Cn1cc(C(C)OCNC(=O)CNC(=O)[C@H](Cc2ccccc2)NC(=O)CNC(=O)CNC(=O)CCCCCN2C(=O)C=CC2=O)nn1] |
| PL37 | 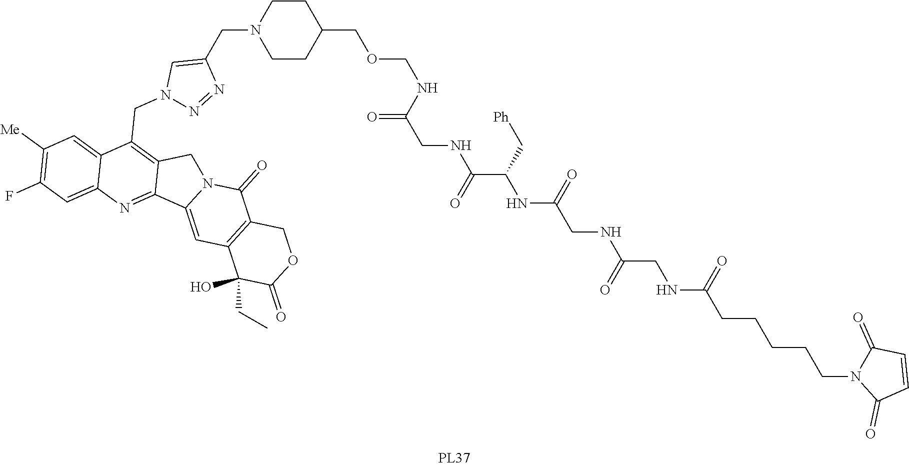 [SMILES: CC[C@@]1(O)C(=O)OCc2c1cc1n(c2=O)Cc2c-1nc1cc(F)c(C)cc1c2Cn1cc(CN2CCC(COCNC(=O)CNC(=O)[C@H](Cc3ccccc3)NC(=O)CNC(=O)CNC(=O)CCCCCN3C(=O)C=CC3=O)CC2)nn1] |
| PL38 | 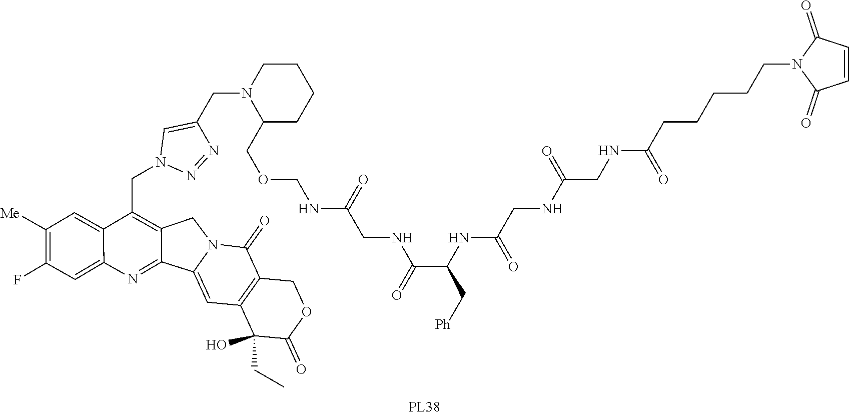 [SMILES: CC[C@@]1(O)C(=O)OCc2c1cc1n(c2=O)Cc2c-1nc1cc(F)c(C)cc1c2Cn1cc(CN2CCCCC2COCNC(=O)CNC(=O)[C@H](Cc2ccccc2)NC(=O)CNC(=O)CNC(=O)CCCCCN2C(=O)C=CC2=O)nn1] |
| PL39 | 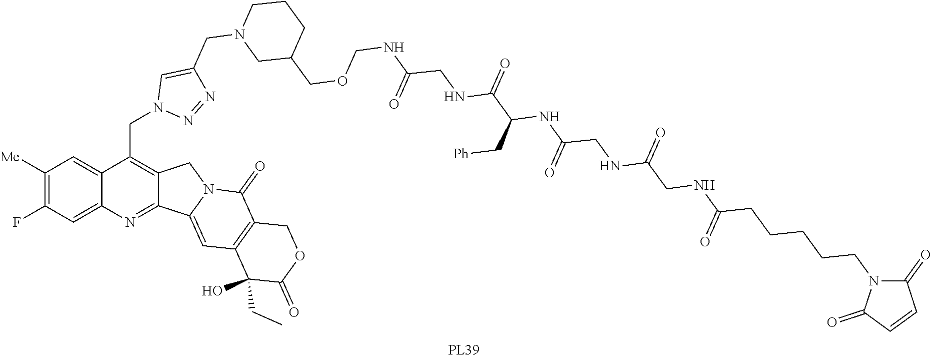 [SMILES: CC[C@@]1(O)C(=O)OCc2c1cc1n(c2=O)Cc2c-1nc1cc(F)c(C)cc1c2Cn1cc(CN2CCCC(COCNC(=O)CNC(=O)[C@H](Cc3ccccc3)NC(=O)CNC(=O)CNC(=O)CCCCCN3C(=O)C=CC3=O)C2)nn1] |

### Example 47: Preparation of ADC

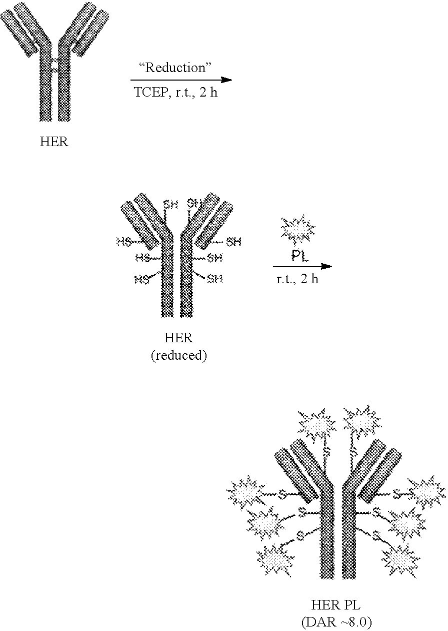

TABLE 04
Synthesis of Conjugates HERPL1-HERPL39.

| | | | DAR | | |
|---|---|---|---|---|---|
| | | | (Drug | Monomeric | |
| | Co-Solvent | Payload | Antibody | Purity | Concentration |
| Conjugates | (%) | Equiv. | Ratio) | (%) | (mg/ml) |
| HERPL1 | 10 | 10 | 7.9 | 99 | 3.7 |
| HERPL2 | 10 | 10 | 7.9 | 99 | 0.4 |
| HERPL3 | No | No | No | No | No |
| | Conjugation | Conjugation | Conjugation | Conjugation | Conjugation |
| HERPL4 | 10 | 15 | 8.0 | 98 | 0.4 |
| HERPL5 | 10 | 10 | 8.0 | 97 | 1.2 |
| HERPL6 | 10 | 20 | 8.0 | 97 | 0.81 |
| HERPL7 | 10 | 10 | 8.0 | 99 | 1.2 |
| HERPL8 | 10 | 20 | 8.5 | 98 | 0.86 |
| HERPL9 | 20 | 20 | 8.0 | 91 | 0.43 |
| HERPL10 | 20 | 20 | 8.6 | 100 | 0.51 |
| HERPL11 | 20 | 10 | 8.0 | 96 | 0.52 |
| HERPL12 | 10 | 10 | 8.0 | 98 | 0.91 |
| HERPL13 | 10 | 15 | 8.0 | 99 | 2.2 |
| HERPL14 | 10 | 15 | 8.0 | 99 | 0.7 |
| HERPL15 | 10 | 10 | 8.0 | 99 | 1.1 |
| HERPL16 | 20 | 15 | 8.0 | 99 | 0.75 |
| HERPL17 | 20 | 15 | 8 | 97 | 2.1 |
| HERPL18 | 20 | 10 | 4.0 | 73 | 1.3 |
| HERPL19 | 20 | 10 | 6.0 | 78 | 0.33 |
| HERPL20 | 20 | 20 | 6.7 | 90 | 0.64 |
| HERPL21 | 20 | 20 | 7.4 | 62 | 0.59 |
| HERPL22 | 10 | 18 | 7.4 | 94 | 2.8 |
| HERPL23 | 10 | 15 | 8.1 | 99 | 2.8 |
| HERPL24 | 10 | 15 | 8.4 | 98 | 2.8 |
| HERPL25 | 10 | 15 | 8.0 | 95 | 1.0 |
| HERPL26 | 10 | 15 | 8.0 | 98 | 2.1 |
| HERPL27 | 20 | 10 | 8.0 | 92 | 1.4 |
| HERPL28 | 10 | 10 | 8.0 | 99 | 1.4 |
| HERPL29 | 10 | 15 | 8.4 | 98 | 1.2 |
| HERPL30 | 10 | 15 | 8.0 | 98 | 2.3 |
| HERPL31 | 10 | 15 | 8.0 | 99 | 1.9 |
| HERPL32 | 20 | 20 | 8.0 | 99 | 2.3 |
| HERPL33 | 10 | | 7.9 | 99 | 3.5 |
| HERPL34 | 10 | 20 | 8.0 | 96 | 0.8 |
| HERPL35 | 10 | 15 | 8.0 | 98 | 1.3 |
| HERPL36 | 10 | 15 | 8.0 | 98 | 1.0 |
| HERPL37 | 20 | 15 | 8.0 | Not | 0.48 |
| | | | | determined | |
| HERPL38 | 10 | 15 | 8.0 | 99 | 0.94 |
| HERPL39 | 10 | 15 | 8.0 | 99 | 0.85 |

### Example 48: Small Scale Conjugation Method (HERPL3-HERPL39)

A stock solution of Herceptin (HER, CAS #AC-NJB-077; 500 mg, 100 mL, 5.1 mg/mL) was prepared in 1×PBS with 1 mM DTPA (pH 6). Tris (2-carboxyethyl) phosphine (TCEP) solution (2.27 mL, 15 mM, 10 eq) was added to the antibody solution (500 mg, 100 mL, 5.1 mg/mL) and allowed to reduce at 37° C. for 90 min. The solution was purified using TFF to remove excess TCEP in presence of 1×PBS with 1 mM DTPA. Aliquoted stock solutions equivalent to 5.1 mg/mL of reduced Herceptin were stored in −80 and thawed as required for each payload-linker conjugation. Solution of PL in DMA (10 μL-15 eq, 10 mM) was added to the 1.0 mg of reduced antibody solution along with 10-20% v/v DMA (25-50 μL) allowed to incubate at room temperature for two hours on rotor for end-to-end mixing. Crude analysis at 2 hours was performed for DAR analysis using RP-LC/MS.

Drug to Antibody Ratio (DAR) by RP-LC/MS was determined by analytical liquid chromatography-mass spectrometry (LC-MS), employing a Waters Acquity Ultra Performance LC system and a Synapt high-definition mass spectrometer. 15 μg of ADC was injected over a PLRP-S column (1000 Å, 8 μm, 2.1×50 mm) against a 25-45% gradient (ACN+0.1% formic acid; 0.35 mL/min flow). Monomeric purity was found via size exclusion chromatography, employed on an Agilent 1260 HPLC. 20 μg of ADC was injected over a TSKgelG3000SWXL column against a 10% IPA in PBS gradient (0.5 mL/min flow). The crude ADC solution was dialyzed using 10K cut-off dialysis cassette generation 3 or passed through 40K Zeba desalting columns against 1×PBS buffer, pH-7.4 to remove free payload, co-solvent and buffer additives. The final solution was filtered using 0.2 μm syringe filter. Final analysis was performed on an aliquoted sample of the filtered ADC. Bioconjugates were reduced with dithiothreitol (0.5 M; 1 μL/10 μg ADC) and allowed to incubate at 37° C.×30 min. Reduced light chain and heavy chain charge envelopes were deconvoluted and reduced light chain and heavy chain species were identified and input into the formula: DAR=[2×(L0+L1)]+[2×(H0+ . . . +H3)]. The calculated DAR of purified ADCs was between 7-8.

### Example 49: Large Scale Conjugation Method (HERPL1& HERPL2)

A stock solution of Herceptin (CAS #180288-69-1; 1 mg, 0.22 mL, 4.5 mg/mL) was prepared in 1×PBS, (pH 7.4). Tris (2-carboxyethyl) phosphine (TCEP) solution (6.7 uL, 10 mM, 10 eq) was added to the antibody solution (1 mg, 0.22 mL, 4.5 mg/mL) with 10% DTPA (23 ul, 10 mM) and allowed to reduce at 37° C. for 90 min. A solution of PL (20 uL-15 eq, 5 mM) was added to the 1.0 mg of reduced antibody solution along with 10% DMA (29 ul) allowed to incubate at room temperature for two hours on rotor for end-to-end mixing. Crude analysis at 2 hours using RP-LC/MS showed DAR 5. An additional bolus of PL (25 uL-19 eq, 5 mM) was added to the reaction solution and allowed to incubate at room temperature for 30 min on rotor for end-to-end mixing to achieve the intended DAR 8.

Drug to Antibody Ratio (DAR) by RP-LC/MS was determined by analytical liquid chromatography-mass spectrometry (LC-MS), employing a Waters Acquity Ultra Performance LC system and a Synapt high-definition mass spectrometer. 15 μg of ADC was injected over a PLRP-S column (1000 Å, 8 μm, 2.1×50 mm) against a 25-45% gradient (ACN+0.1% formic acid; 0.35 mL/min flow). Monomeric purity was found via size exclusion chromatography, employed on an Agilent 1260 HPLC. 20 μg of ADC was injected over a TSKgelG3000SWXL column against a 100% PBS gradient (0.8 mL/min flow). The Her-PL crude ADC solution was dialyzed against 1×PBS buffer, pH-7.4 using 0.5 ml dialysis cassette generation 2 at room temperature for 5-6 h followed up overnight at cold room with continuous stirring using magnetic stirrer to remove free payload, buffer additives, and TCEP. The final solution was filtered using 0.2 μm syringe filter. Final analysis was performed on an aliquoted sample of the filtered ADC. Bioconjugates were reduced with dithiothreitol (0.5 M; 1 μL/10 μg ADC) and allowed to incubate at 37° C.×30 min. Reduced light chain and heavy chain charge envelopes were deconvoluted and reduced light chain and heavy chain species were identified and input into the formula: DAR=[2×(L0+L1)]+[2×(H0+ . . . +H3)]

### Example 50: Cytotoxicity Assay

### Protocol:

Day 1: 2,000 cells/well were plated and incubated at 37° C. overnight.

Day 2: Payload/ADC/vehicle control dilutions were made in respective media and added to cells. Volume added: 50 uL to each well. Starting treatment concentration was 1 mM for free payloads/1 μM for ADC's/10% for vehicle control and then diluted 10-fold down for a total of 11 treatment dilutions. Each concentration was analyzed in triplicates. Media-only wells were used as a control to calculate percent viability.

Day 5: Cell titer glow reagent (volume: 50 uL) was added to each well, the plate was shaken for 5 mins and the luminescence was recorded. Drug Treatment Time: 72 hours. Data Analysis: Percent viability was calculated by dividing the luminescence signal obtained for each treated well by the untreated well (media-only control) and multiplying by 100. Data was next transformed using X=Log (x) and then analyzed with nonlinear regression (curve fit), Dose Response inhibition−log (inhibitor) vs response (3 parameters) using PRISM software to determine the IC₅₀ value. The results of cytotoxicity of SKBR-3 cell line (human breast cancer) and are provided in Tables 05 and 06.

TABLE 05
In Vitro Cytotoxicity Assay of Payloads 1-69.

| | IC₅₀ (nM) | IC₅₀ (nM) (DXD Control) | Normalized IC₅ₒ (PL |
|---|---|---|---|
| Payload | SKBR-3 | (1599440-33-1) SKBR-3 | IC₅₀/DXD IC₅₀) |
| 1 | 77.79 | 8.971 | 8.67 |
| 2 | 80.86 | 8.971 | 9.01 |
| 3 | 107.4 | 8.971 | 11.97 |
| 4 | 125.9 | 8.971 | 14.03 |
| 5 | 81.47 | 4.29 | 18.99 |
| 6 | 690.1 | 7.27 | 94.92 |
| 7 | 1830 | 8.971 | 203.99 |
| 8 | 16.49 | 8.971 | 1.84 |
| 9 | 94.57 | 7.27 | 13.01 |
| 11 | 194 | 4.29 | 45.22 |
| 12 | 374 | 8.971 | 41.69 |
| 13 | 30.82 | 4.508 | 6.84 |
| 15 | 25.8 | 0.9285 | 27.79 |
| 16 | 38.54 | 4.508 | 8.55 |
| 19 | 22.36 | 4.508 | 4.96 |
| 22 | 102 | 5.096 | 20.02 |
| 23 | 1247 | 6.25 | 199.52 |
| 32 | 335.7 | 15.3 | 21.94 |
| 33 | 32.88 | 1.432 | 22.96 |
| 36 | 1010 | 4.96 | 203.63 |
| 37 | 50.8 | 15.3 | 3.32 |
| 38 | 34.57 | 0.9285 | 37.23 |
| 39 | 4.63 | 5.01 | 0.92 |
| 46 | 3.68 | 15.3 | 0.24 |
| 47 | 33.16 | 6.82 | 4.86 |
| 48 | 40.25 | 6.25 | 6.44 |
| 49 | 8.14 | 6.25 | 1.30 |
| 50 | 18.65 | 5.01 | 3.72 |
| 51 | 15.3 | 5.01 | 3.05 |
| 52 | 17.98 | 6.25 | 2.88 |
| 53 | 18.02 | 5.01 | 3.60 |
| 54 | 8.94 | 1.432 | 6.24 |
| 55 | 25.2 | 1.432 | 17.60 |
| 56 | 10.09 | 1.432 | 7.05 |
| 57 | 18.33 | 1.432 | 12.80 |
| 58 | 30.54 | 1.284 | 23.79 |
| 59 | 4.03 | 0.8322 | 4.84 |
| 60 | 284.5 | 1.189 | 338.2 |
| 61 | 211.2 | 1.189 | 177.62 |
| 62 | 2039.0 | 1.189 | 1714.88 |
| 63 | 41.05 | 0.9285 | 44.21 |
| 64 | 20.62 | 0.9285 | 22.21 |
| 65 | 134.46 | 7.27 | 18.50 |
| 66 | 54.71 | 15.3 | 3.58 |
| 67 | 9.15 | 15.3 | 0.60 |
| 68 | 17.65 | 0.9285 | 19.01 |
| 69 | 95.75 | 0.9195 | 104.13 |

TABLE 06
In Vitro Cytotoxicity Assay of ADCs HERPL1-HERPL39.

| | | IC_{5O} (pM) | |
|---|---|---|---|
| | | (Trastuzumab- | Normalized IC₅ₒ |
| | IC₅₀ (pM) | Deruxtecan Control) | (HERPL/HERDXD) |
| Conjugates | SKBR-3 | SKBR-3 | SKBR-3 |
| HERPL1 | 43.75 | 59.28 | 0.74 |
| HERPL2 | 169.8 | 121.1 | 1.40 |
| HERPL4 | 62 | 139 | 0.45 |
| HERPL5 | 423.5 | 270 | 1.57 |
| HERPL6 | 120.3 | 75.61 | 1.59 |
| HERPL7 | 126.4 | 84.16 | 1.50 |
| HERPL8 | 36.81 | 56.9 | 0.65 |
| HERPL9 | 95.89 | 75.61 | 1.27 |
| HERPL10 | 113 | 139 | 0.81 |
| HERPL11 | 989 | 139 | 7.12 |
| HERPL12 | 172.7 | 84.16 | 2.05 |
| HERPL13 | 531.6 | 121.1 | 4.39 |
| HERPL14 | 531 | 84.16 | 6.31 |
| HERPL15 | 494.6 | 270.4 | 1.83 |
| HERPL16 | 415.9 | 270.4 | 1.54 |
| HERPL17 | 436.6 | 59.26 | 7.37 |
| HERPL18 | 139.3 | 84.16 | 1.66 |
| HERPL19 | 2199 | 56.9 | 38.6 |
| HERPL20 | 2100 | 139 | 15.1 |
| HERPL21 | 2721 | 56.9 | 47.7 |
| HERPL22 | 55.38 | 40.47 | 1.37 |
| HERPL23 | 98.55 | 40.47 | 2.44 |
| HERPL24 | 69.46 | 40.47 | 1.72 |
| HERPL25 | 164.3 | 145.8 | 1.13 |
| HERPL26 | 666 | 95 | 7.01 |
| HERPL27 | 128.9 | 40.47 | 3.18 |
| HERPL28 | 60.74 | 40.47 | 1.50 |
| HERPL29 | 33.76 | 145.8 | 0.23 |
| HERPL30 | 99.19 | 145.8 | 0.68 |
| HERPL31 | 252.2 | 73.07 | 3.45 |
| HERPL32 | 606.8 | 73.07 | 8.30 |
| HERPL33 | 38.66 | 59.28 | 0.65 |
| HERPL34 | 92.66 | 75.61 | 1.23 |
| HERPL35 | 117.2 | 59.61 | 1.97 |
| HERPL36 | 55.86 | 59.61 | 0.94 |
| HERPL37 | 372.1 | 56.9 | 6.54 |
| HERPL38 | 541.7 | 84.16 | 6.44 |
| HERPL39 | 704.8 | 84.16 | 8.37 |

SEQUENCE LISTING

SEQ ID NO: 1
GGFG

Organism: artificial
Comment: amino acid spacer

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, reaction products, and the like. Furthermore, the terms “first,” “second,” and the like, herein do not denote any order, quantity, or importance, but rather are used to denote one element from another. The terms “a” and “an” and “the” herein do not denote a limitation of quantity and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the group(s) includes one or more groups). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A compound of Formula (1), or a pharmaceutically acceptable salt thereof, 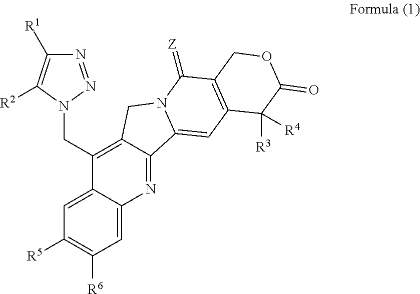
wherein
Z is O or S;
each of R¹ and R² independently is H, optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-;
or
each of R¹ and R² independently is Y⁴—Y³—Y²—Y¹—, wherein
Y¹ is a bond, aryl, heteroaryl, or C₁₋₃ alkyl;
Y² is a bond, O, S, N, or NH, wherein when Y² is N then each of R¹ and R² independently is (Y⁴—Y³)₂—N—Y¹—, or the N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, R², Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH— or H₂N—(C₁₋₂ alkyl)-(C═O)—NH—;
each of R³, R⁴, R⁵, and R⁶ independently is H, optionally substituted C₁₋₆ alkyl, C₁₋₆ haloalkyl, C₁₋₆ alkoxy, halogen, C₂-C₆ alkanoyl, C₁₋₆ alkylthio, cyano, nitro, hydroxyl, amino, mono-C₁₋₆ alkylamine, di-C₁₋₆ alkylamine, optionally substituted C₃-C₈ cycloalkyl, optionally substituted C₄-C₈ cycloalkenyl, optionally substituted aryl, optionally substituted heteroaryl, or optionally substituted C₃-C₇ heterocyclic, wherein each optionally substituted group of R³, R⁴, R⁵, or R⁶ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—; and
further wherein any O, S, or N is optionally protected with a suitable protecting group.

2. The compound of claim 1, wherein R⁵ is C₁₋₆ alkyl.

3. The compound of claim 1, wherein R⁶ is halogen.

4. The compound of claim 1, wherein R³ is hydroxyl, C₁₋₃ alkyl, or C₁ haloalkyl.

5. The compound of claim 1, wherein R⁴ is C₁₋₃ alkyl or C₁₋₃ haloalkyl.

6. The compound of claim 1 of Formula (1a), or a pharmaceutically acceptable salt thereof,

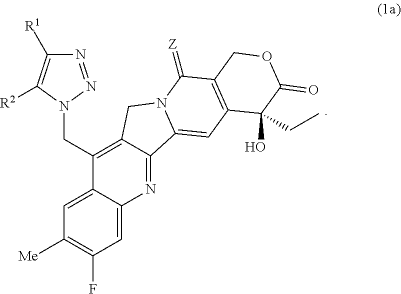

7. The compound of claim 1, wherein R² is H and R¹ is optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-;
or
R¹ is Y⁴—Y³—Y²—Y¹—, wherein
Y¹ is a bond, aryl, heteroaryl, or C₁₋₃ alkyl;
Y² is a bond, O, S, N, or NH, wherein when Y² is N then R¹ is (Y⁴—Y³)₂—N—Y¹—, or the N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—; and
further wherein any O, S, or N is optionally protected with a suitable protecting group.

8. The compound of claim 1, wherein R¹ is H and R² is optionally substituted C₁₋₆ alkyl, C₁₋₆ haloalkyl, C₂-C₆ alkanoyl, COOH, optionally substituted aryl, optionally substituted heteroaryl, HO—(C₁₋₂ alkyl)-, or H₂N—(C₁₋₂ alkyl)-;
or
R² is Y⁴—Y³—Y²—Y¹—, wherein
Y¹ is a bond, aryl, heteroaryl, or C₁₋₃ alkyl;
Y² is a bond, O, S, N, or NH, wherein when Y² is N then R² is (Y⁴—Y³)₂—N—Y¹—, or the N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted aryl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH—, or H₂N—(C₁₋₂ alkyl)-(C═O)—NH—; and
further wherein any O, S, or N is optionally protected with a suitable protecting group.

9. The compound of claim 1, wherein one of R¹ and R² is H and the other is HO—(C₁₋₂ alkyl)-, H₂N—(C₁₋₂ alkyl)-, optionally substituted phenyl, or Y⁴—Y³—Y²—Y¹—, wherein
Y¹ is a bond, aryl, heteroaryl, or C₁₋₃ alkyl;
Y² is a bond, O, N, or NH, wherein when Y² is N then N forms an optionally substituted C₅-C₇ heterocyclic with Y⁴;
Y³ is a bond or C═O; and
Y⁴ is H, hydroxyl, amino, mono-C₁₋₆ alkylamine, di-C₁₋₆ alkylamine, optionally substituted C₁₋₆ alkyl, C₁₋₆ haloalkyl, C₁₋₆ alkoxy, C₂-C₆ alkanoyl, C₁₋₆ alkylthio, COOH, optionally substituted C₃-C₈ cycloalkyl, optionally substituted C₄-C₈ cycloalkenyl, optionally substituted phenyl, optionally substituted heteroaryl, optionally substituted C₃-C₇ heterocyclic, or an amino acid;
or Y⁴ together with Y² form an optionally substituted C₅-C₇ heterocyclic;
wherein each optionally substituted group of R¹, R², Y², and Y⁴ independently is optionally substituted with 1 or 2 substituents, wherein each substituent independently is hydroxyl, amino, COOH, C₁₋₆ alkyl, C₁₋₆ haloalkyl, C₁₋₆ alkoxy, C₂-C₆ alkanoyl, C₁₋₆ alkylthio, cyano, halogen, nitro, mono-C₁₋₆ alkylamine, di-C₁₋₆ alkylamine, C₃-C₈ cycloalkyl, C₄-C₈ cycloalkenyl, aryl, heteroaryl, C₃-C₇ heterocyclic, C₂₋₆ alkenyl, C₂₋₆ alkynyl, HO—(C₁₋₂ alkyl)-, H₂N—(C₁₋₂ alkyl)-, HO—(C₁₋₂ alkyl)-(C═O)—, H₂N—(C₁₋₂ alkyl)-(C═O)—, HO—(C₁₋₂ alkyl)-(C═O)—NH— or H₂N—(C₁₋₂ alkyl)-(C═O)—NH—.

10. A compound from Table 01.

11. A compound of Formula (A) or Formula (B) 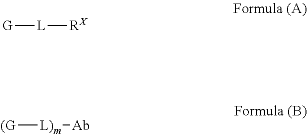
wherein
L is a linking group;
G is a structure of Formula (1) of claim 1 or a structure found in Table 01, covalently attached to L through one of R¹, R², R³, R⁴, R⁵, or R⁶;
R^{X} is a reactive group suitable for forming a covalent bond to an antibody or antibody fragment;
Ab is an antibody or antibody fragment; and
m is 1, 2, 3, 4, 5, 6, 7, or 8.

12. The compound of claim 11, wherein L, the linking group, is a bond or a group containing 1 to about 250 non-hydrogen atoms including C, N, O, S, halogen, or a combination thereof, further wherein L can optionally include one or more groups including an ether, thioether, amide, carbonyl, ester, carbonate, carbamate, urea, or a combination thereof.

13. The compound of claim 12, wherein L comprises an ethylene glycol unit; an amino acid unit; or a combination thereof.

14. The compound of claim 13, wherein the amino acid groups of L, each amino acid unit can be arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, or citrulline (Cit).

15. The compound of claim 11, wherein R^{X} is an amine, —O—NH₂, maleimide, azide, 2,5-dioxopyrrolidin-1-yl formate, 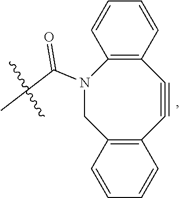 [SMILES: *C(=O)N1Cc2ccccc2C#Cc2ccccc21]
thiol, or pentafluorophenyl ester.

16. The compound of claim 11, wherein -L-R^{X} is a structure from Table 02; or
a compound from Table 03.

17. A pharmaceutical formulation comprising, the compound of claim 1 and a pharmaceutically acceptable excipient.

18. A method of treating a proliferative disease including a cancer, comprising administering to a patient in need thereof the compound of claim 1 optionally formulated as a pharmaceutical formulation.

19. A conjugate from Table 04.
